SEQUENCE LISTING (50 sequences)

SEQ ID NO: 1 - protein, 138 aa, synthetic construct
  REGION 1..138: note = SYNTHETIC CONSTRUCT
MSKNKDQRTAKTLERTWDTLNHLLFISSCLYKLNLKSVAQITLSILAIVISTSLIIAAIIFIASANHKVTPTTAIIQDATSQIKNTPPSKPNNDFHFEVFNFVPCSICSNNPTCWAICKRIPNKKPGKKTTTKPTKKP

SEQ ID NO: 2 - DNA, 417 bp, synthetic construct
  misc_feature 1..417: note = SYNTHETIC CONSTRUCT
  source 1..417: mol_type = other DNA
atgagcaagaacaaggaccagcggaccgccaagacactggaaagaacctgggacaccctgaaccatctgctgttcatcagcagctgcctgtacaagctgaacctgaagtctgtggcccagatcaccctgagcatcctggccatcgtgatcagcaccagcctgatcattgccgccatcatctttatcgccagcgccaaccacaaagtgacccctaccacagccatcatccaggacgccacaagccagatcaagaacacccctccaagcaagcccaacaacgacttccacttcgaggtgttcaacttcgtgccctgcagcatctgcagcaacaatcctacctgctgggccatctgcaagagaatccccaacaagaagcccggcaaaaagaccaccacaaagcccacaaagaagccctag

SEQ ID NO: 3 - protein, 206 aa, synthetic construct
  REGION 1..206: note = SYNTHETIC CONSTRUCT
MSKNKDQRTTKTLEKTWDTLNHLLFISSCLYKLNLKSIAQITLSILAMIISTSLIIAAIIFIASANHKVTLTTAIIQDATSQIKNTTPTYLTQNPQLGISFSNLSETTSQTTTILASTTPSVKSTLQSTTVKTKNTTTTKIQPSKPTTKQRQNKPPNKPNNDFHFEVFNFVPCSICSNNPTCWAICKRIPNKKPGKKTTTKPTKKP

SEQ ID NO: 4 - DNA, 618 bp, synthetic construct
  misc_feature 1..618: note = SYNTHETIC CONTRUCT
  source 1..618: mol_type = other DNA
atgtccaaaaacaaggaccaacgcaccaccaagacactagaaaagacctgggacactctcaatcatctattattcatatcatcgtgcttatacaagttaaatcttaaatctatagcacaaatcacattatccattctggcaatgataatctcaacttcacttataattgcagccatcatattcatagcctcggcaaaccacaaagtcacactaacaactgcaatcatacaagatgcaacaagccagatcaagaacacaaccccaacatacctcacccagaatccccagcttggaatcagcttctccaatctgtctgaaactacatcacaaaccaccaccatactagcttcaacaacaccaagtgtcaagtcaaccctgcaatccacaacagtcaagaccaaaaacacaacaacaaccaaaatacaacccagcaagcccaccacaaaacaacgccaaaacaaaccaccaaacaaacccaataatgattttcactttgaagtgttcaactttgtaccttgcagcatatgcagcaacaatccaacctgctgggctatctgtaaaagaataccaaacaaaaaacctggaaagaaaaccaccaccaagcccacaaaaaaacca

SEQ ID NO: 5 - protein, 206 aa, synthetic construct
  REGION 1..206: note = SYNTHETIC CONSTRUCT
MSKNKDQRTAKTLERTWDTLNHLLFISSCLYKLNLKSVAQITLSILAIVISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKP

SEQ ID NO: 6 - DNA, 621 bp, synthetic construct
  misc_feature 1..621: note = SYNTHETIC CONSTRUCT
  source 1..621: mol_type = other DNA
atgagcaagaacaaggaccagcggaccgccaagacactggaaagaacctgggacaccctgaaccatctgctgttcatcagcagctgcctgtacaagctgaacctgaagtctgtggcccagatcaccctgagcatcctggccatcgtgatcagcaccagcctgatcattgccgccatcatctttatcgccagcgccaaccacaaagtgacccctaccacagccatcatccaggacgccacaagccagatcaagaacaccacacctacctacctgacacagaaccctcagctgggcatcagccctagcaatcctagcgagatcacctctcagatcaccacaatcctggccagcacaacccctggcgtgaagtctacactgcagagcaccaccgtgaaaacgaagaataccaccaccacacagacccagcctagcaagcctaccaccaagcagagacagaacaagcctccaagcaagcccaacaacgacttccacttcgaggtgttcaacttcgtgccctgcagcatctgcagcaacaatcctacctgctgggccatctgcaagagaatccccaacaagaagcccggcaaaaagaccaccacaaagcccacaaagaagccctag

SEQ ID NO: 7 - protein, 298 aa, synthetic construct
  REGION 1..298: note = SYNTHETIC CONSTRUCT
MSKNKDQRTTKTLEKTWDTLNHLLFISSCLYKLNLKSIAQITLSILAMIISTSLIIAAIIFIASANHKVTLTTAIIQDATSQIKNTTPTYLTQNPQLGISFSNLSETTSQTTTILASTTPSVKSTLQSTTVKTKNTTTTKIQPSKPTTKQRQNKPPNKPNNDFHFEVFNFVPCSICSNNPTCWAICKRIPNKKPGKKTTTKPTKKPTIKTTKKDLKPQTTKPKEVPTTKPTEKPTINTTKTNIRTTLLTNNTTGNPEHTSQKGTLHSTSSDGNPSPSQVYTTSEYLSQPPSPSNTTNQ

SEQ ID NO: 8 - DNA, 837 bp, synthetic construct
  misc_feature 1..837: note = SYNTHETIC CONSTRUCT
  source 1..837: mol_type = other DNA
atgtccaaaaacaaggaccaacgcaccaccaagacactagaaaagaaatctatagcacaaatcacattatccattctggcaatgataatctcaacttcacttataattgcagccatcatattcatagcctcggcaaaccacaaagtcacactaacaactgcaatcatacaagatgcaacaagccagatcaagaacacaaccccaacatacctcacccagaatccccagcttggaatcagcttctccaatctgtctgaaactacatcacaaaccaccaccatactagcttcaacaacaccaagtgtcaagtcaaccctgcaatccacaacagtcaagaccaaaaacacaacaacaaccaaaatacaacccagcaagcccaccacaaaacaacgccaaaacaaaccaccaaacaaacccaataatgattttcactttgaagtgttcaactttgtaccttgcagcatatgcagcaacaatccaacctgctgggctatctgtaaaagaataccaaacaaaaaacctggaaagaaaaccaccaccaagcccacaaaaaaaccaaccatcaagacaaccaaaaaagatctcaaacctcaaaccacaaaaccaaaggaagtacctaccaccaagcccacagaaaagccaaccatcaacaccaccaaaacaaacatcagaactacactgctcaccaacaataccacaggaaatccagaacacacaagtcaaaagggaaccctccactcaacctcctccgatggcaatccaagcccttcacaagtctatacaacatccgagtacctatcacaacctccatctccatccaacacaacaaaccagtag

SEQ ID NO: 9 - protein, 298 aa, synthetic construct
  REGION 1..298: note = SYNTHETIC CONSTRUCT
MSKNKDQRTAKTLERTWDTLNHLLFISSCLYKLNLKSVAQITLSILAIVISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 10 - DNA, 897 bp, synthetic construct
  misc_feature 1..897: note = SYNTHETIC CONSTRUCT
  source 1..897: mol_type = other DNA
atgagcaagaacaaggaccagcggaccgccaagacactggaaagaacctgggacaccctgaaccatctgctgttcatcagcagctgcctgtacaagctgaacctgaagtctgtggcccagatcaccctgagcatcctggccatcgtgatcagcaccagcctgatcattgccgccatcatctttatcgccagcgccaaccacaaagtgacccctaccacagccatcatccaggacgccacaagccagatcaagaacaccacacctacctacctgacacagaaccctcagctgggcatcagccctagcaatcctagcgagatcacctctcagatcaccacaatcctggccagcacaacccctggcgtgaagtctacactgcagagcaccaccgtgaaaacgaagaataccaccaccacacagacccagcctagcaagcctaccaccaagcagagacagaacaagcctccaagcaagcccaacaacgacttccacttcgaggtgttcaacttcgtgccctgcagcatctgcagcaacaatcctacctgctgggccatctgcaagagaatccccaacaagaagcccggcaaaaagaccaccacaaagcccacaaagaagcccacactgaaaaccaccaagaaggaccccaagcctcagaccaccaagtccaaagaggtgcccaccaccaaacctaccgaggaacccaccatcaacaccactaagaccaacatcatcaccacactgctgacctccaacaccaccggcaatcctgaactgaccagccagatggaaaccttccacagcacctccagcgagggcaacccatctcctagtcaggtgtccaccacaagcgagtaccctagccagccaagcagccctcctaacacacctagacagtag

SEQ ID NO: 11 - protein, 138 aa, synthetic construct
  REGION 1..138: note = SYNTHETIC CONSTRUCT
MSKNKDQRTAKTLERTWDTLNHLLFISSCLYKLNLKSVAQITLSILAIVISTSLIIAAIIFIASANHKVTPTTAIIQDATSQIKNTPPSKPNNDFHFEVFNFVPCSICSNNPTCWAIACKRIPNKPGKKTTTKPTKKP

SEQ ID NO: 12 - DNA, 420 bp, synthetic construct
  misc_feature 1..420: note = SYNTHETIC CONSTRUCT
  source 1..420: mol_type = other DNA
atgagcaagaacaaggaccagcggaccgccaagacactggaaagaacctgggacaccctgaaccatctgctgttcatcagcagctgcctgtacaagctgaacctgaagtctgtggcccagatcaccctgagcatcctggccatcgtgatcagcaccagcctgatcattgccgccatcatctttatcgccagcgccaaccacaaagtgacccctaccacagccatcatccaggacgccacaagccagatcaagaacacccctccaagcaagcccaacaacgacttccacttcgaggtgttcaacttcgtgccctgcagcatctgcagcaacaatcctacctgctgggccatcgcctgcaagagaatccccaacaagaagcccggcaaaaagaccaccacaaagcccacaaagaagccctag

SEQ ID NO: 13 - protein, 299 aa, synthetic construct
  REGION 1..299: note = SYNTHETIC CONSTRUCT
MSKNKDQRTAKTLERTWDTLNHLLFISSCLYKLNLKSVAQITLSILAIVISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAIACKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 14 - DNA, 900 bp, synthetic construct
  misc_feature 1..900: note = SYNTHETIC CONSTRUCT
  source 1..900: mol_type = other DNA
atgagcaagaacaaggaccagcggaccgccaagacactggaaagaacctgggacaccctgaaccatctgctgttcatcagcagctgcctgtacaagctgaacctgaagtctgtggcccagatcaccctgagcatcctggccatcgtgatcagcaccagcctgatcattgccgccatcatctttatcgccagcgccaaccacaaagtgacccctaccacagccatcatccaggacgccacaagccagatcaagaacaccacacctacctacctgacacagaaccctcagctgggcatcagccctagcaatcctagcgagatcacctctcagatcaccacaatcctggccagcacaacccctggcgtgaagtctacactgcagagcaccaccgtgaaaacgaagaataccaccaccacacagacccagcctagcaagcctaccaccaagcagagacagaacaagcctccaagcaagcccaacaacgacttccacttcgaggtgttcaacttcgtgccctgcagcatctgcagcaacaatcctacctgctgggccatcgcctgcaagagaatccccaacaagaagcccggcaaaaagaccaccacaaagcccacaaagaagcccacactgaaaaccaccaagaaggaccccaagcctcagaccaccaagtccaaagaggtgcccaccaccaaacctaccgaggaacccaccatcaacaccactaagaccaacatcatcaccacactgctgacctccaacaccaccggcaatcctgaactgaccagccagatggaaaccttccacagcacctccagcgagggcaacccatctcctagtcaggtgtccaccacaagcgagtaccctagccagccaagcagccctcctaacacacctagacagtag

SEQ ID NO: 15 - protein, 138 aa, synthetic construct
  REGION 1..138: note = SYNTHETIC CONSTRUCT
MSKHKNQRTARTLEKTWDTLNHLIVISSCLYRLNLKSIAQIALSVLAMIISTSLIIAAIIFIISANHKVTLTTVTVQTIKNHTEKNPPKKPKDDYHFEVFNFVPCSICGNNQLCKSICKTIPSNKPKKKPTIKPTNKP

SEQ ID NO: 16 - DNA, 417 bp, synthetic construct
  misc_feature 1..417: note = SYNTHETIC CONSTRUCT
  source 1..417: mol_type = other DNA
atgtccaaacacaagaatcaacgcactgccaggactctagaaaagacctgggatactctcaatcatctaattgtaatatcctcttgtttatacagattaaatttaaaatctatagcacaaatagcactatcagttctggcaatgataatctcaacctctctcataattgcagccataatattcatcatctctgccaatcacaaagttacactaacaacggtcacagttcaaacaataaaaaaccacactgaaaaaaacccaccaaaaaaaccaaaagatgattaccattttgaagtgttcaacttcgttccctgtagtatatgtggcaacaatcaactttgcaaatccatctgtaaaacaataccaagcaacaaaccaaagaagaaaccaaccatcaaacccacaaacaaaccatag

SEQ ID NO: 17 - protein, 138 aa, synthetic construct
  REGION 1..138: note = SYNTHETIC CONSTRUCT
MSKHKNQRTARTLEKTWDTLNHLIVISSCLYRLNLKSIAQIALSVLAIVISTSLIIAAIIFIISANHKVTLTTVTVQTIKNHTEKNPPKKPKDDYHFEVFNFVPCSICGNNQLCKSICKTIPSNKPKKKPTIKPTNKP

SEQ ID NO: 18 - DNA, 417 bp, synthetic construct
  misc_feature 1..417: note = SYNTHETIC CONSTRUCT
  source 1..417: mol_type = other DNA
atgtccaaacacaagaatcaacgcactgccaggactctagaaaagacctgggatactctcaatcatctaattgtaatatcctcttgtttatacagattaaatttaaaatctatagcacaaatagcactatcagttctggcaatcgtgatctcaacctctctcataattgcagccataatattcatcatctctgccaatcacaaagttacactaacaacggtcacagttcaaacaataaaaaaccacactgaaaaaaacccaccaaaaaaaccaaaagatgattaccattttgaagtgttcaacttcgttccctgtagtatatgtggcaacaatcaactttgcaaatccatctgtaaaacaataccaagcaacaaaccaaagaagaaaccaaccatcaaacccacaaacaaaccatag

SEQ ID NO: 19 - protein, 299 aa, synthetic construct
  REGION 1..299: note = SYNTHETIC CONSTRUCT
MSKHKNQRTARTLEKTWDTLNHLIVISSCLYRLNLKSIAQIALSVLAMIISTSLIIAAIIFIISANHKVTLTTVTVQTIKNHTEKNITTYLTQVPPERVSSSKQPTTTSPIHTNSATTSPNTKSETHHTTAQTKGRTTTSTQTNKPSTKPRLKNPPKKPKDDYHFEVFNFVPCSICGNNQLCKSICKTIPSNKPKKKPTIKPTNKPTTKTTNKRDPKTPAKTTKKETTTNPTKKPTLTTTERDTSTSQSTVLDTTTLEHTIQQQSLHSTTPENTPNSTQTPTASEPSTSNSTQNTQSHA

SEQ ID NO: 20 - DNA, 900 bp, synthetic construct
  misc_feature 1..900: note = SYNTHETIC CONSTRUCT
  source 1..900: mol_type = other DNA
atgtccaaacacaagaatcaacgcactgccaggactctagaaaagacctgggatactctcaatcatctaattgtaatatcctcttgtttatacagattaaatttaaaatctatagcacaaatagcactatcagttctggcaatgataatctcaacctctctcataattgcagccataatattcatcatctctgccaatcacaaagttacactaacaacggtcacagttcaaacaataaaaaaccacactgaaaaaaacatcaccacctaccttactcaagtcccaccagaaagggttagctcatccaaacaacctacaaccacatcaccaatccacacaaattcagccacaacatcacccaacacaaagtcagaaacacaccacacaacagcacaaaccaaaggcagaaccaccacctcaacacagaccaacaagccgagcacaaaaccacgcctaaaaaatccaccaaaaaaaccaaaagatgattaccattttgaagtgttcaacttcgttccctgtagtatatgtggcaacaatcaactttgcaaatccatctgtaaaacaataccaagcaacaaaccaaagaagaaaccaaccatcaaacccacaaacaaaccaaccaccaaaaccacaaacaaaagagacccaaaaacaccagccaaaacgacgaaaaaagaaactaccaccaacccaacaaaaaaaccaaccctcacgaccacagaaagagacaccagcacctcacaatccactgtgctcgacacaaccacattagaacacacaatccaacagcaatccctccactcaaccacccccgaaaacacacccaactccacacaaacacccacagcatccgagccctctacatcaaattccacccaaaatacccaatcacatgcttag

SEQ ID NO: 21 - protein, 299 aa, synthetic construct
  REGION 1..299: note = SYNTHETIC CONSTRUCT
MSKHKNQRTARTLEKTWDTLNHLIVISSCLYRLNLKSIAQIALSVLAIVISTSLIIAAIIFIISANHKVTLTTVTVQTIKNHTEKNITTYLTQVPPERVSSSKQPTTTSPIHTNSATTSPNTKSETHHTTAQTKGRTTTSTQTNKPSTKPRLKNPPKKPKDDYHFEVFNFVPCSICGNNQLCKSICKTIPSNKPKKKPTIKPTNKPTTKTTNKRDPKTPAKTTKKETTTNPTKKPTLTTTERDTSTSQSTVLDTTTLEHTIQQQSLHSTTPENTPNSTQTPTASEPSTSNSTQNTQSHA

SEQ ID NO: 22 - DNA, 900 bp, synthetic construct
  misc_feature 1..900: note = SYNTHETIC CONSTRUCT
  source 1..900: mol_type = other DNA
atgtccaaacacaagaatcaacgcactgccaggactctagaaaagacctgggatactctcaatcatctaattgtaatatcctcttgtttatacagattaaatttaaaatctatagcacaaatagcactatcagttctggcaatcgtgatctcaacctctctcataattgcagccataatattcatcatctctgccaatcacaaagttacactaacaacggtcacagttcaaacaataaaaaaccacactgaaaaaaacatcaccacctaccttactcaagtcccaccagaaagggttagctcatccaaacaacctacaaccacatcaccaatccacacaaattcagccacaacatcacccaacacaaagtcagaaacacaccacacaacagcacaaaccaaaggcagaaccaccacctcaacacagaccaacaagccgagcacaaaaccacgcctaaaaaatccaccaaaaaaaccaaaagatgattaccattttgaagtgttcaacttcgttccctgtagtatatgtggcaacaatcaactttgcaaatccatctgtaaaacaataccaagcaacaaaccaaagaagaaaccaaccatcaaacccacaaacaaaccaaccaccaaaaccacaaacaaaagagacccaaaaacaccagccaaaacgacgaaaaaagaaactaccaccaacccaacaaaaaaaccaaccctcacgaccacagaaagagacaccagcacctcacaatccactgtgctcgacacaaccacattagaacacacaatccaacagcaatccctccactcaaccacccccgaaaacacacccaactccacacaaacacccacagcatccgagccctctacatcaaattccacccaaaatacccaatcacatgcttag

SEQ ID NO: 23 - protein, 574 aa, synthetic construct
  REGION 1..574: note = SYNTHETIC CONSTRUCT
MELLILKANAITTILTAVTFCFASGQNITEEFYQSTCSAVSKGYLSALRTGWYTSVITIELSNIKKNKCNGTDAKIKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 24 - DNA, 1812 bp, synthetic construct
  misc_feature 1..1812: note = SYNTHETIC CONSTRUCT
  source 1..1812: mol_type = other DNA
cggaaggcccatgaggccagttaattaagaggtaccggatccgccaccatggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaaacaagtgcaacggcaccgacgccaagatcaagctgatcaagcaggaactggacaagtacaagaacgccgtgaccgagctgcagctgctgatgcagagcacccccgccaccaacaaccgggctagacgcgagctgcctcggttcatgaactacaccctgaacaacgccaaaaagaccaacgtgaccctgagcaagaagcggaagcggcggttcctgggcttcctgctgggcgtgggcagcgccattgctagcggagtggccgtgtgcaaggtgctgcacctggaaggcgaagtgaacaagatcaagtccgccctgctgagcaccaacaaggccgtggtgtccctgagcaacggcgtgtccgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactgactcgagctcatggcgcgcctaggccttgacggccttccg

SEQ ID NO: 25 - DNA, 1725 bp, synthetic construct
  misc_feature 1..1725: note = SYNTHETIC CONSTRUCT
  source 1..1725: mol_type = other DNA
atggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaaacaagtgcaacggcaccgacgccaagatcaagctgatcaagcaggaactggacaagtacaagaacgccgtgaccgagctgcagctgctgatgcagagcacccccgccaccaacaaccgggctagacgcgagctgcctcggttcatgaactacaccctgaacaacgccaaaaagaccaacgtgaccctgagcaagaagcggaagcggcggttcctgggcttcctgctgggcgtgggcagcgccattgctagcggagtggccgtgtgcaaggtgctgcacctggaaggcgaagtgaacaagatcaagtccgccctgctgagcaccaacaaggccgtggtgtccctgagcaacggcgtgtccgtgctgaccttcaaggtgctggatctgaagaactacatcgacaagcagctgctgcccatcctgaacaagcagagctgcagcatcagcaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgtgcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactga

SEQ ID NO: 26 - protein, 552 aa, synthetic construct
  REGION 1..552: note = SYNTHETIC CONSTRUCT
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 27 - DNA, 1746 bp, synthetic construct
  misc_feature 1..1746: note = SYNTHETIC CONSTRUCT
  source 1..1746: mol_type = other DNA
cggaaggcccatgaggccagttaattaagaggtaccggatccgccaccatggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaatcaagtgcaacggcaccgacgccaagatcaagctgatcaagcaggaactggacaagtacaagaacgccgtgaccgagctgcagctgctgatgcagagcacccccgccaccaacaaccaggctagaggcagcggaagcggacggtccctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactgactcgagctcatggcgcgcctaggccttgacggccttccg

SEQ ID NO: 28 - DNA, 1659 bp, synthetic construct
  misc_feature 1..1659: note = SYNTHETIC CONSTRUCT
  source 1..1659: mol_type = other DNA
atggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaatcaagtgcaacggcaccgacgccaagatcaagctgatcaagcaggaactggacaagtacaagaacgccgtgaccgagctgcagctgctgatgcagagcacccccgccaccaacaaccaggctagaggcagcggaagcggacggtccctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactga

SEQ ID NO: 29 - protein, 552 aa, synthetic construct
  REGION 1..552: note = SYNTHETIC CONSTRUCT
MELLILKANAITTILTAVTFCFASGQNITEEFYQSTCSAVSKGYLSALRTGWYTSVITIELSNIKKIKCNGTDAKIKLIKQELDKYKNAVTELQLLMQSTPATNNQARGSGSGRSLGFLLGVGSAIASGVAVSKVLHLEGEVNKIKSALLSTNKAVVSLSNGVSVLTSKVLDLKNYIDKQLLPIVNKQSCSIPNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDQFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 30 - DNA, 1746 bp, synthetic construct
  misc_feature 1..1746: note = SYNTHETIC CONSTRUCT
  source 1..1746: mol_type = other DNA
cggaaggcccatgaggccagttaattaagaggtaccggatccgccaccatggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaatcaagtgcaacggcaccgacgccaagatcaagctgatcaagcaggaactggacaagtacaagaacgccgtgaccgagctgcagctgctgatgcagagcacccccgccaccaacaaccaggctagaggcagcggaagcggacggtccctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgaccagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactgactcgagctcatggcgcgcctaggccttgacggccttccg

SEQ ID NO: 31 - DNA, 1659 bp, synthetic construct
  misc_feature 1..1659: note = SYNTHETIC CONSTRUCT
  source 1..1659: mol_type = other DNA
atggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaatcaagtgcaacggcaccgacgccaagatcaagctgatcaagcaggaactggacaagtacaagaacgccgtgaccgagctgcagctgctgatgcagagcacccccgccaccaacaaccaggctagaggcagcggaagcggacggtccctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatccccaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgaccagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactga

SEQ ID NO: 32 - protein, 114 aa, synthetic construct
  REGION 1..114: note = SYNTHETIC CONSTRUCT
MELLILKANAITTILTAVTFCFASGQNITEEFYQSNEKINQSLAFIRKSDELLHNVNAVKSTTNIMITTIIIVIIVILLSLIAVGLLLYCKARSTPVTLSKDQLSGINNIAFSN

SEQ ID NO: 33 - DNA, 345 bp, synthetic construct
  misc_feature 1..345: note = SYNTHETIC CONSTRUCT
  source 1..345: mol_type = other DNA
atggaactgctgatcctgaaggccaacgccatcaccaccatcctgaccgctgtgaccttctgcttcgccagcggccagaacatcaccgaggaattctaccagagcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactga

SEQ ID NO: 34 - protein, 574 aa, synthetic construct
  REGION 1..574: note = SYNTHETIC CONSTRUCT
MELPILKTNAITAILAAVTLCFASSQNITEEFYQTTCSAVSKGYLSALRTGWYTSVITIELSNIKENKCNGTDAKVKLIKQELDKYKSAVTELQLLMQSTPATNNRARRELPRFMNYTLNNTKNTNVTLSKKRKRRFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTINIMITTIIIVIIVILLSLIAVGLLLYCKARSTPVTLSKDQLSGINNIAFSN

SEQ ID NO: 35 - DNA, 2142 bp, synthetic construct
  misc_feature 1..2142: note = SYNTHETIC CONSTRUCT
  source 1..2142: mol_type = other DNA
atggagttgccaatcctcaaaacaaatgcaattaccgcaatccttgctgcagtcacactctgttttgcttccagtcaaaacatcactgaagaattttatcaaacaacatgcagtgcagtcagcaaaggctatcttagtgctctaagaactggttggtatactagtgttataactatagaattaagtaatatcaaggaaaataagtgtaatggaacagacgctaaggtaaaattgataaaacaagaattagataaatataaaagtgctgtaacagaattgcagttgctcatgcaaagcacaccggcaaccaacaatcgagccagaagagaactaccaaggtttatgaattatacactcaacaataccaaaaataccaatgtaacattaagcaagaaaaggaaaagaagatttcttggctttttgttaggtgttggatctgcaatcgccagtggcattgctgtatctaaggtcctgcacctagaaggggaagtgaacaaaatcaaaagtgctctactatccacaaacaaggctgtagtcagcttatcaaatggagttagtgtcttaaccagcaaagtgttagacctcaaaaactatatagataaacagttgttacctattgtgaacaagcaaagctgtagcatatcaaacattgaaactgtgatagagttccaacaaaagaacaacagactactagagattaccagggaatttagtgttaatgcaggtgtaactacacctgtaagcacttatatgttaacaaatagtgaattattatcattaatcaatgatatgcctataacaaatgatcagaaaaagttaatgtccaacaatgttcaaatagttagacagcaaagttactctatcatgtccataataaaggaggaagtcttagcatatgtagtacaattaccactatatggtgtaatagatacaccttgttggaaactgcacacatcccctctatgtacaaccaacacaaaggaagggtccaacatctgtttaacaagaaccgacagaggatggtactgtgacaatgcaggatcagtatctttcttcccactagctgaaacatgtaaagttcaatcgaatcgagtattttgtgacacaatgaacagtttaacattaccaagtgaagtaaatctctgcaacattgacatattcaaccccaaatatgattgcaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataagggggttgacactgtgtctgtaggtaatacattatattatgtaaataagcaagaaggcaaaagtctctatgtaaaaggtgaaccaataataaatttctatgacccattagtgttcccctctgatgaatttgatgcatcaatatctcaagtcaatgagaagattaaccagagcctagcatttattcgtaaatccgatgaattattacataatgtaaatgctggtaaatccaccataaatatcatgataactactataattatagtgattatagtaatattgttatcattaattgccgttggactgctcctatactgcaaggccagaagcacaccagtcacactaagcaaggatcaactgagtggtataaataatattgcatttagtaactaaaacgggtgcgattatgtatcaaataagggggttgacactgtgtctgtaggtaatacattatattatgtaaataagcaagaaggcaaaagtctctatgtaaaaggtgaaccaataataaatttctatgacccattagtgttcccctctgatgaatttgatgcatcaatatctcaagtcaatgagaagattaaccagagcctagcatttattcgtaaatccgatgaattattacataatgtaaatgctggtaaatccaccataaatatcatgataactactataattatagtgattatagtaatattgttatcattaattgccgttggactgctcctatactgcaaggccagaagcacaccagtcacactaagcaaggatcaactgagtggtataaataatattgcatttagtaactaa

SEQ ID NO: 36 - protein, 574 aa, synthetic construct; identical to SEQ ID NO: 34
  REGION 1..574: note = SYNTHETIC CONSTRUCT

SEQ ID NO: 37 - protein, 574 aa, synthetic construct
  REGION 1..574: note = SYNTHETIC CONSTRUCT
MELLILKANAITTILTAVTFCFASGQNITEEFYQSTCSAVSKGYLSALRTGWYTSVITIELSNIKKNKCNGTDAKIKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAVKSTTNIMITTIIIVIIVILLSLIAVGLLLYCKARSTPVTLSKDQLSGINNIAFSN

SEQ ID NO: 38 - DNA, 1725 bp, synthetic construct
  misc_feature 1..1725: note = SYNTHETIC CONSTRUCT
  source 1..1725: mol_type = other DNA
atggaactgctgatcctgaaggccaacgccatcaccaccatcctgaccgctgtgaccttctgcttcgccagcggccagaacatcaccgaggaattctaccagagcacctgtagcgccgtgtccaagggctacctgagcgccctgcggaccggctggtacaccagcgtgatcaccatcgagctgagcaacatcaagaaaaacaagtgcaacggcaccgacgccaagatcaagctgatcaagcaggaactggacaagtacaagaacgccgtgaccgagctgcagctgctgatgcagagcacccccgccaccaacaaccgggctagacgcgagctgcctcggttcatgaactacaccctgaacaacgccaaaaagaccaacgtgaccctgagcaagaagcggaagcggcggttcctgggcttcctgctgggcgtgggcagcgccattgctagcggagtggccgtgtcaaaggtgctgcacctggaaggcgaagtgaacaagatcaagtccgccctgctgagcaccaacaaggccgtggtgtccctgagcaacggcgtgtccgtgctgaccagcaaggtgctggatctgaagaactacatcgacaagcagctgctgcccatcgtgaacaagcagagctgcagcatcagcaacatcgagacagtgatcgagttccagcagaagaacaaccggctgctggaaatcacccgcgagttcagcgtgaacgccggcgtgaccacccccgtgtccacctacatgctgaccaacagcgagctgctgagcctgatcaacgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgataatgccggcagcgtgtcattctttccacaagccgagacatgcaaggtgcagagcaaccgggtgttctgcgacaccatgaacagcctgaccctgccctccgaagtgaacctgtgcaacgtggacatcttcaaccctaagtacgactgcaagatcatgacctccaagaccgacgtgtccagctccgtgatcacctccctgggcgccatcgtgtcctgctacggcaagaccaagtgcaccgccagcaacaagaaccggggcatcatcaagaccttcagcaacggctgcgactacgtgtccaacaagggggtggacaccgtgtccgtgggcaacaccctgtactacgtgaacaaacaggaaggcaagagcctgtacgtgaagggcgagcccatcatcaacttctacgaccccctggtgttccccagcgacgagttcgacgccagcatcagccaggtcaacgagaagatcaaccagagcctggccttcatcagaaagagcgacgagctgctgcacaatgtgaatgccgtgaagtccaccaccaatatcatgatcaccacaatcatcatcgtgatcatcgtcatcctgctgtccctgatcgccgtgggcctgctgctgtactgcaaggcccggtccacccctgtgaccctgtccaaggaccagctgagcggcatcaacaatatcgccttctccaactga

SEQ ID NO: 39 - protein, 256 aa, synthetic construct
  REGION 1..256: note = SYNTHETIC CONSTRUCT
METYVNKLHEGSTYTAAVQYNVLEKDDDPASLTIWVPMFQSSMPADLLIKELANVNILVKQISTPNGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 40 - DNA, 771 bp, synthetic construct
  misc_feature 1..771: note = SYNTHETIC CONSTRUCT
  source 1..771: mol_type = other DNA
atggaaacatacgtgaacaaacttcacgaaggctccacatacacagctgctgttcaatacaatgtcttagaaaaagacgatgaccctgcatcacttacaatatgggtgcccatgttccaatcatccatgccagcagatttacttataaaagaactagctaatgtcaacatactagtgaaacaaatatccacacccaatggaccttcattaagagtcatgataaactcaagaagtgcagtgctagcacaaatgcccagcaaatttaccatatgtgccaatgtgtccttggatgaaagaagcaagctggcatatgatgtaaccacaccctgtgaaatcaaggcatgtagtctaacatgcctaaaatcaaaaaatatgttaactacagttaaagatctcactatgaaaacactcaacccaacacatgacatcattgctttatgtgaatttgaaaatatagtaacatcaaaaaaagtcataataccaacatacctaagatccatcagtgtcagaaataaagatctgaacacacttgaaaatataacaaccactgaattcaaaaatgccatcacaaatgcaaaaatcatcccttactcaggattactgttagtcatcacagtgactgacaacaaaggagcattcaaatacataaagccacaaagtcaatttatagtagatcttggagcttacctagaaaaagaaagtatatattatgttacaacaaattggaagcacacagctacacgatttgcaatcaaacccatggaagattaa

SEQ ID NO: 41 - protein, 259 aa, synthetic construct
  REGION 1..259: note = SYNTHETIC CONSTRUCT
MFQSSMPADLLIKDSTYTAAVQYNVLEKDDDPASLTIWVPMFQSSMPADLLIKELANVNILVKQISTPKGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 42 - DNA, 780 bp, synthetic construct
  misc_feature 1..780: note = SYNTHETIC CONSTRUCT
  source 1..780: mol_type = other DNA
atgttccagagcagcatgcccgccgacctgctgatcaaagacagcacctacacagccgccgtgcagtacaacgtgctggaaaaggacgacgaccccgccagcctgaccatctgggtgcccatgttccagagcagcatgcccgccgacctgctgatcaaagaactggccaacgtgaacatcctggtcaagcagatcagcacccccaagggccccagcctgagagtgatgatcaacagccgcagcgccgtgctggcccagatgcccagcaagttcaccatctgcgccaacgtgtccctggacgagcggagcaagctggcctacgacgtgaccaccccctgcgagatcaaggcctgcagcctgacctgcctgaagtccaagaacatgctgaccaccgtgaaggacctgaccatgaagaccctgaaccccacccacgacatcattgccctgtgcgagttcgagaacatcgtgaccagcaagaaagtgatcatccccacctacctgcggagcatcagcgtgcggaacaaggacctgaacaccctggaaaacatcaccaccaccgagttcaagaacgccattaccaacgccaagatcatcccctacagcggcctgctgctggtcatcaccgtgaccgacaacaagggcgccttcaagtacatcaagccccagagccagttcatcgtggacctgggcgcctacctggaaaaagaatccatctactacgtcaccaccaactggaagcacaccgccaccagattcgccatcaagcccatggaagattga

SEQ ID NO: 43 - protein, 194 aa, synthetic construct
  REGION 1..194: note = SYNTHETIC CONSTRUCT
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEEPNSPKIRVYNTVISYIESNRKNNKQTIHLLKRLPADVLKKTIKTTLDIHKSITINNPKESTVSDINDHAKNNDTT

SEQ ID NO: 44 - DNA, 585 bp, synthetic construct
  misc_feature 1..585: note = SYNTHETIC CONSTRUCT
  source 1..585: mol_type = other DNA
atgtcacgaaggaatccttgcaaatttgaaattcgaggtcattgcttgaatggtaagaggtgtcattttagtcataattattttgaatggccaccccatgcactgcttgtaagacaaaactttatgttaaacagaatacttaagtctatggataaaagcatcgatactttatcagaaataagtggagctgcagagttggacagaacagaagagtatgccctcggtgtagttggagtgctagagagttatataggatctataaataatataactaaacaatcagcatgtgttgccatgagcaaactcctcactgaactcaacagtgatgacatcaaaaaactgagggacaatgaagagccaaattcacccaagataagagtgtacaatactgtcatatcatatattgaaagcaacaggaaaaacaataaacaaactatccatctgttaaaaagattgccagcagacgtattgaagaaaaccataaaaaccacattggatatccacaagagcataaccatcaataacccaaaagaatcaactgttagtgatataaacgaccatgccaaaaataatgatactacctga

SEQ ID NO: 45 - protein, 194 aa, synthetic construct
  REGION 1..194: note = SYNTHETIC CONSTRUCT
MSRRNPCKFEIRGHCLNGKRCHFSHNYFEWPPHALLVRQNFMLNRILKSMDKSIDTLSEISGAAELDRTEEYALGVVGVLESYIGSINNITKQSACVAMSKLLTELNSDDIKKLRDNEELNSPKIRVYNTVISYIESNRKNNKQTIHLLKRLPADVLKKTIKNTLDIHKSITINNPKESTVSDTNDHAKNNDTT

SEQ ID NO: 46 - DNA, 585 bp, synthetic construct
  misc_feature 1..585: note = SYNTHETIC CONSTRUCT
  source 1..585: mol_type = other DNA
atgagccggcggaacccctgcaagttcgagatccggggccactgcctgaacggcaagcggtgccacttcagccacaactacttcgagtggccccctcacgccctgctggtgcgccagaacttcatgctgaaccggatcctgaagtccatggacaagagcatcgacaccctgagcgagatcagcggagctgccgagctggaccggaccgaggaatatgccctgggcgtggtgggagtgctggaaagctacatcggcagcatcaacaacatcaccaagcagagcgcctgcgtggccatgagcaagctgctgaccgagctgaacagcgacgacatcaagaagctgcgggacaacgaggaactgaacagccccaagatccgggtgtacaacaccgtgatcagctacatcgagagcaaccggaagaacaacaagcagaccatccatctgctgaagcggctgcccgccgacgtgctgaagaaaaccatcaagaacaccctggacatccacaagtccatcaccatcaacaaccccaaagaaagcaccgtgtccgacaccaacgaccacgccaagaacaacgacaccacctga

SEQ ID NO: 47 - protein, 241 aa, synthetic construct
  REGION 1..241: note = SYNTHETIC CONSTRUCT
MEKFAPEFHGEDANNRATKFLESIKGKFTSPKDPKKKDSIISVNSIDIEVTKESPITSNSTIINPTNETDDTVGNKPNYQRKPLVSFKEDPTPSDNPFSKLYKETIETFDNNEEESSYSYEEINDQTNDNITARLDRIDEKLSEILGMLHTLVVASAGPTSARDGIRDAMVGLREDMIEKIRTEALMTNDRLEAMARLRNEESEKMAKDTSDEVSLNPTSEKLNNLLEGNDSDNDLSLDDF

SEQ ID NO: 48 - DNA, 726 bp, synthetic construct
  misc_feature 1..726: note = SYNTHETIC CONSTRUCT
  source 1..726: mol_type = other DNA
atggaaaagtttgctcctgaattccatggagaagatgcaaacaacagagctaccaaattcctagaatcaataaagggcaaattcacatcacctaaagatcccaagaaaaaagatagtatcatatctgtcaactcaatagatatagaagtaaccaaagaaagccctataacatcaaattcaaccattataaacccaacaaatgagacagatgatactgtagggaacaagcccaattatcaaagaaaacctctagtaagtttcaaagaagaccctacgccaagtgataatcccttttcaaaactatacaaagaaaccatagaaacatttgataacaatgaagaagaatctagctattcatatgaagaaataaatgatcagacaaacgataatataacagcaagattagataggattgatgaaaaattaagtgaaatactaggaatgcttcacacattagtagtagcgagtgcaggacctacatctgctcgggatggtataagagatgccatggttggtttaagagaagacatgatagaaaaaatcagaactgaagcattaatgaccaatgacagactagaagctatggcaagactcaggaatgaggaaagtgaaaagatggcaaaagacacatcagatgaagtgtctctcaatccaacatcagagaaattgaacaacctgttggaagggaatgatagtgacaatgatctatcacttgatgatttctga

SEQ ID NO: 49 - protein, 241 aa, synthetic construct
  REGION 1..241: note = SYNTHETIC CONSTRUCT
MEKFAPEFHGEDANNRATKFLESIKGKFTSPKDPKKKDSIISVNSIDIEVTKESPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMIGLREEMIEKIRTEALMTNDRLEAMARLRNEESEKMAKDTSDEVSLNPTSEKLNNLLEGNDSDNDLSLEDF

SEQ ID NO: 50 - DNA, 726 bp, synthetic construct
  misc_feature 1..726: note = SYNTHETIC CONSTRUCT
  source 1..726: mol_type = other DNA
atggaaaagttcgcccccgagttccacggcgaggacgccaacaaccgggccaccaagtttctggaatccatcaagggcaagttcaccagccccaaggaccccaagaagaaggacagcatcatcagcgtgaacagcatcgacatcgaagtgaccaaagagagccccatcaccagcaacagcaccatcatcaaccccaccaacgagacagacgacaccgccggcaacaagcccaactaccagcggaagcccctggtgtccttcaaagaggaccccacccccagcgacaaccccttcagcaagctgtacaaagagacaatcgagacattcgacaacaacgaggaagagagcagctacagctacgaggaaatcaacgaccagaccaacgacaacatcaccgccagactggaccggatcgacgagaagctgagcgagatcctgggcatgctgcacaccctggtggtggcctctgccggccctacaagcgccagagatggcatccgggacgccatgatcggcctgcgggaagagatgatcgagaagatccggaccgaggccctgatgaccaacgaccggctggaagccatggcccggctgcggaacgaggaatccgagaagatggccaaggacaccagcgacgaggtgtccctgaaccccacctctgagaagctgaacaacctgctggaaggcaacgacagcgacaacgacctgagcctggaagatttctga

(19) United States
(12) Patent Application Publication
(10) Pub. No.: US 2026/0000750 A1
(43) Pub. Date: Jan. 1, 2026

(54) RSV VIRUS-LIKE PARTICLES AND METHODS OF USE THEREOF

(71) Applicants: EMORY UNIVERSITY, Atlanta, GA (US); CHILDREN’S HEALTHCARE OF ATLANTA, Atlanta, GA (US); WISCONSIN ALUMNI RESEARCH FOUNDATION, Madison, GA (US)

(72) Inventors: Binh HA, Atlanta, GA (US); Larry J. ANDERSON, Atlanta, GA (US); Elizabeth R. WRIGHT, Atlanta, GA (US)

(21) Appl. No.: 19/262,633

(22) Filed: Jul. 8, 2025

Related U.S. Application Data
(62) Division of application No. 17/289,827, filed on Apr. 29, 2021, now Pat. No. 12,370,252.
(60) Provisional application No. 62/751,975, filed on Oct. 29, 2018.

(51) Int. Cl. A61K 39/155; A61K 39/39; C12N 7/00

(52) U.S. Cl. CPC A61K 39/155; A61K 39/39; C12N 7/00; C12N 2760/18023; C12N 2760/18034

(57) ABSTRACT

The present disclosure relates to virus-like particles and vaccine compositions for inducing immunity and preventing respiratory syncytial virus (RSV) infection.

### CROSS REFERENCE TO RELATED APPLICATIONS

This application is a Divisional of U.S. patent application Ser. No. 17/289,827, filed Apr. 29, 2021, which depends from and claims priority to PCT/US2019/058559 filed Oct. 29, 2019, which claims priority to U.S. Provisional Patent Application Ser. No. 62/751,975 filed Oct. 29, 2018, the disclosures of which are expressly incorporated herein by reference.

### REFERENCE TO SEQUENCE LISTING

A Sequence Listing conforming to the rules of WIPO Standard ST.26 is hereby incorporated by reference. Said Sequence Listing has been filed as an electronic document via Patent Center in ASCII format encoded as XML. The electronic document, created on Jul. 2, 2025, is entitled “10029-068US2_ST26.xml”, and is 96,636 bytes in size.

### FIELD

The present disclosure relates to virus-like particles and uses thereof.

### BACKGROUND

Respiratory Syncytial Virus (RSV) was quickly recognized as an important pediatric pathogen after its discovery in the 1950s. It causes upper and lower respiratory tract infections including bronchitis, bronchiolitis, and pneumonia. Most children are infected by 2 years of age. However, since its infection provides incomplete protection, RSV infects throughout life with the elderly and persons with chronic cardiac or pulmonary disease, or immune compromising conditions at higher risk for severe complications. It is estimated that globally there are more than 33 million episodes of RSV infections and 95,000-150,000 RSV deaths, mostly in developing countries, in children <5 years of age. RSV-related deaths are rare in the United States; it is, however, responsible for an estimated 60,000-170,000 hospitalizations each year in children <5 years of age. Also, infants hospitalized with RSV infection are prone to later development of obstructive airway diseases and asthma. Its substantial global disease burden has made RSV a high priority for vaccine and anti-viral drug development. There are, however, no effective anti-viral drugs or vaccines yet available. Therefore, what is needed is a vaccine for inducing protective immunity to RSV infection. The compositions and methods disclosed herein address these and other needs.

### SUMMARY

Disclosed herein are virus like-particles (VLPs) for use in inducing immunity to respiratory syncytial virus (RSV) infections or symptoms thereof.

In some aspects, disclosed herein is a virus like particle (VLP) comprising a respiratory syncytial virus (RSV) M protein and an RSV M2-1 protein.

In some embodiments, the VLP comprises one or more additional RSV proteins. In some embodiments, the VLP comprises an RSV F protein. In some embodiments, the RSV F protein is selected from a group consisting of a pre-fusion form of the RSV F protein, a post-fusion form of the RSV F protein, and a carbonyl terminal portion of the RSV F protein. In some embodiments, the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37. In some embodiments, the carbonyl terminal portion of the RSV F protein comprises a sequence of SEQ ID NO: 32.

In some embodiments, the VLP comprises an RSV G protein. In some embodiments, the RSV G protein is from RSV group A or RSV group B. In some embodiments, the RSV G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21.

In some embodiments, the VLP comprises a recombinant RSV G protein. In some embodiments, the recombinant RSV G protein comprises a transmembrane domain of an RSV G protein and a central conserved domain of an RSV G protein.

In some aspects, disclosed herein is a virus like particle (VLP) comprising a respiratory syncytial virus (RSV) M protein, an RSV P protein, an RSV F protein, and an RSV G protein.

In some embodiments, the RSV F protein is selected from a group consisting of a pre-fusion form of the RSV F protein, a post-fusion form of the RSV F protein, and a carbonyl terminal portion of the RSV F protein. In some embodiments, the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37. In some embodiments, the carbonyl terminal portion of the RSV F protein comprises a sequence of SEQ ID NO: 32.

In some embodiments, the RSV G protein is from RSV group A or RSV group B. In some embodiments, the RSV G protein is from RSV group A and RSV group B. In some embodiments, the RSV G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21.

In some embodiments, the RSV G protein is a recombinant RSV G protein. In some embodiments, the VLP comprises a recombinant RSV G protein. In some embodiments, the recombinant RSV G protein comprises a transmembrane domain of the RSV G protein and a central conserved domain of the RSV G protein.

In some aspects, disclosed herein is a vaccine comprising the VLP of any preceding aspect. In some embodiments, the vaccine further comprises an adjuvant.

In some aspects, disclosed herein is a method of inducing an immunological response to RSV infection or at least one symptom thereof in a subject, comprising administering one or more effective doses of the vaccine of any preceding aspect. In some embodiments, the one or more effective doses of the vaccine are administered to the subject via a route that is selected from the group consisting of an intramuscular route, a subcutaneous route, an intradermal route, an oral administration, a nasal administration, and inhalation.

### BRIEF DESCRIPTION OF THE DRAWINGS

The accompanying figures, which are incorporated in and constitute a part of this specification, illustrate several aspects described below.

FIGS. 1A-1C show generation and expression of F and G on RSV VLPs. 293F cell line expressing RSV gene M, F (or Fₜ), G (or G_{P}) and P or M2-1 were induced for 72 h in 2 μg/ml doxycycline. Cells were harvested and low centrifugation performed to separate cells and VLPs-containing supernatant. VLPs were filtered through 0.45 μm filter to clear cell debris, layered on top of 20% sucrose and subjected to centrifugation at 12,200×g for 2 h, 4° C. VLP pellets were resuspended in sterile PBS and subjected to centrifugation through a 20-60% sucrose gradient at 11,000×g for 12 h, 4° C. A total of 10 fractions were collected and analyzed by immunoblotting using 3D3 (human anti-G antibody), motavizumab (human anti-F antibody), and rabbit serum anti-M antibody. FIG. 1A shows VLPs MFGP and MFGM2-1. FIG. 1B shows VLPs MFG_{P}P and MFG_{P}M2-1. FIG. 1C shows MGP, MFP, and MFₜGP VLPs. GP, truncated G: aa 1-86+155-206. Fₜ, truncated F: aa 496-574.

FIGS. 2A and 2B show that glycoproteins are visualized as spikes on VLPs by negative stain electron microscopy. FIG. 2A shows that a 3 μl aliquot containing the diluted sample was applied for 1 min onto a formvar/carbon coated, 300 mesh-copper grid that has been glow discharged for 30 sec, then negatively stained with 0.75% freshly-made uranyl formate on ice for 1 min. Data were collected using a FEI T20 electron microscope operating at 200 kV (pixel size 1.101 A, total electron dose is 54 electrons/A square)”. FIG. 2B shows that MFGP VLPs were labeled with 3D3 or motavizumab followed by incubating with gold-labeled anti human secondary antibody.

FIGS. 3A-3C show that immunized animals generate serum anti-G and anti-F antibodies. FIG. 3A shows schematic schedule of animal experiments. FIG. 3B shows that sera from immunized animals (diluted 1:200) were used in binding ELISA to immobilize 293F cell lysate containing Ga, Gb, or F antigen. After blocking, plates were incubated with goat anti mouse IgG-HRP secondary antibody. OPD substrate was used to develop reaction and absorbance at 490 nm was read. FIG. 3C shows that sera from immunized animals were heat inactivated at 56° C. for 30 min followed by 2-fold serial dilution in triplicates. The dilutions were incubated with 100 TCID50 of RSV A2 virus for 1 h at RT. The mixtures were then transferred to monolayer HEp-2 cell and incubated for 1 h at 37° C. in 5% CO₂. 5% FBS+MEM media was added to the cells followed by incubation for 72 h at 37° C. in 5% CO₂. Cells were fixed and ELISA was performed using goat anti-RSV antibody and HPR-conjugated donkey anti-goat secondary antibody. Reaction was developed by OPD and absorbance read at 490 nm. Neutralizing titers were calculated using Reed-Muench method.

FIG. 4 shows that immunized animals have significant less lung viral titer. Lungs from immunized animals were homogenized as described in materials and methods. Aliquots stored at −80° C. were thawed and total RNA was extracted from lung. RNAs were then reverse transcribed into cDNAs. These were used as templates in RT-PCR using CYBR green and a pair of RSV matrix protein M specific primers as described. In parallel, similar reactions were performed using a pair of B-actin specific primers as controls. Results were expressed as relative amount of RSV M compared to β-actin. * p<0.05.

FIGS. 5A and 5B shows that immunized animals had significant less lung mucin. Female BALB/c mice (4-6 weeks) were divided in 7 groups (n=4), immunized, and challenged as summarized in table 2. Lungs were collected, fixed, and stained with Periodic-acid Schiff (PAS) staining as described in materials and methods. The slides were analyzed by Aperio ImageScope software and scored blindly on a 0-4 scale and subsequently converted to a 0-100% histopathology scale. FIG. 5A shows representative images from corresponding groups. FIG. 5B shows quantitative data converted from histopathology scale. * p<0.05, ** p<0.01.

FIG. 6 shows domains of Ga protein, Gb protein, Fpost protein and Fpre protein and combination of F and G expression of RSV VLPs using platforms M+P and M+M2-1.

FIG. 7 shows G and/or F expression of RSV VLPs. 293F cells expressing VLPs were induced for 72 h. Cell supernatants were collected, centrifuged, purified through 0.45 μm filter and 20% sucrose at 10,000×g for 2 h, 4° C. Pellets resuspended in PBS as well as solubilized cells were analyzed by immunoblotting with anti-G (3D3) and anti-F (motavizumab) antibodies. S: supernatant; C, cells.

FIG. 8 shows that glycoproteins are visualized as spikes on VLPs by negative stain electron microscopy. A 3 μl aliquot containing the diluted sample was applied for 1 minute onto a formvar/carbon coated, 300 mesh-copper grid that has been glow discharged for 3 seconds, then negatively stained with 0.75% freshly-made uranyl formate on ice for 1 minute. Data were collected using a FEI T20 electron microscope operating at 200 kV (pixel size 1.101 A, total electron dose is 54 electrons/A square).

FIG. 9 shows a schematic of examples of RSV F proteins. DS-Cav1: this structure-based design generates a stabilized prefusion F structure that retains antigenic site Ø (recognized by the prefusion specific antibody D25). This is achieved by creating a double-mutation at amino acids S155C and S290C forming a stable F trimer. That combines with S190F and V207L hydrophobic pair mutations that fill a cavity in the structure creating DS-Cav1. SC-DM and SC-TM are both designed to have a short linker between F1 and F2 subunits of the F protein. Further mutations in the F secondary structure to limit transformation from prefusion to postfusion conformation at N671 and S215P creates SC-DM. Additional mutation at E487Q to minimize negative repulsion generates SC-TM.

### DETAILED DESCRIPTION

RSV is a single-stranded, negative sense RNA virus belonging to Paramyxoviridae family and Pneumoviridae subfamily with two distinct antigenic groups, A and B. The RSV genome of approximately 15.2 kb includes ten genes that encode for eleven proteins. Three proteins, the fusion (F), attachment (G), and small hydrophobic (SH) are expressed on the virion envelop. The F and G proteins are the only proteins shown to induce effective neutralizing antibodies and longer-term protective immunity. The F protein is more conserved among RSV strains and induces cross-protective immunity and is most effective at inducing neutralizing antibodies. The SH protein does induce some protection likely through Fc receptor-mediated activity such as antibody dependent cellular cytotoxicity or complement activation. Most neutralizing antibodies in human serum specimens are against the pre-fusion form of F and pre-fusion F is currently a prime candidate for RSV vaccines. The G protein, though eliciting less potent neutralizing antibody, has been shown to be an important factor for RSV disease pathogenesis making it also a candidate for inclusion in an RSV vaccine. The G protein structure consists of a conserved region that contains a CX3C chemokine motif that enables binding to the CX3C chemokine receptor, CX3CR1, and has some activities similar to the CX3C chemokine fractalkine. The G protein induces disease causing inflammatory responses that can be inhibited by blocking G binding to CX3CR1 with passive administration of an anti-G monoclonal antibody, G peptide vaccine induced antibodies, or by mutating the CX3C motif. Thus, a vaccine that induces both anti-F and anti-G antibodies can decrease disease by both decreasing viral replication and producing an anti-inflammatory effect.

Despite over 60 years of research, no effective vaccine or antiviral drug is available. Studies, however, show most neutralizing antibodies produced after RSV infection are against the surface fusion F and attachment G glycoproteins, thus making these the prime candidates for vaccines. In fact, antibodies induced by the F protein reduce viral titers. Antibodies induced by the G protein are less effective at reducing viral titers than those induced by F but reduce inflammation and disease more effectively. Among different strategies for RSV vaccines, virus-like particles (VLPs) are safe, immunogenic, and used in licensed human vaccines.

Disclosed herein is a vaccine that includes both the F and G proteins utilizing M based virus-like particles (VLPs). An RSV-based vaccine platform is optimal since all components can contribute to vaccine-induced immunity and the protein structures in the VLP better reflect natural structures. A VLP that better reflects the structure in the virus can induce a more effective immune response and provide a better model for structural studies. RSV VLPs have previously been developed using the Newcastle disease, influenza, or bacterial phage P22 platforms with the RSV F and/or G proteins or M and M2 proteins.

Disclosed herein are novel RSV VLPs with F and/or G proteins using an RSV platform with M plus P or M plus M2-1. Since M2-1 is important for structural stability of RSV, M2-1 can also contribute to VLP formation. The data herein show that an RSV-based vaccine platform can be used to efficiently form RSV VLPs with either M plus P or M plus M2-1 with F and/or G proteins. Unexpectedly, the inventors found that the G protein was only efficiently incorporated into these VLPs in the presence of the intracellular and transmembrane domains of F.

Described herein are virus like-particles (VLPs) and methods for inducing immunity to respiratory syncytial virus (RSV) infections or symptoms thereof. In one aspect, disclosed herein is a virus like particle (VLP) comprising a respiratory syncytial virus (RSV) M protein and an RSV M2-1 protein, wherein the VLP further comprises an RSV F protein and/or an RSV G protein. In one aspect, disclosed herein is a virus like particle (VLP) comprising a respiratory syncytial virus (RSV) M protein, an RSV P protein, an RSV F protein, and an RSV G protein.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

As used herein the term “adjuvant” refers to a compound that, when used in combination with a specific immunogen in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

A “composition” is intended to include a combination of active agent and another compound or composition, inert (for example, a detectable agent or label) or active, such as an adjuvant.

As used herein, the term “virus-like particle” (VLP) refers to a structure that in at least one attribute resembles a virus but which has not been demonstrated to be infectious. In general, virus-like particles lack a viral genome and, therefore, are noninfectious. In addition, virus-like particles can often be produced in large quantities by heterologous expression and can be easily purified.

As used herein, the term “vaccine” refers to a formulation which contains VLPs of the present invention, which is in a form that is capable of being administered to a subject and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of CD8+ T cells, antigen presenting cells, CD4+ T cells, dendritic cells and/or other cellular responses.

As used herein an “effective dose” generally refers to that amount of VLPs or vaccines of the invention sufficient to induce immunity, to prevent and/or ameliorate an infection or to reduce at least one symptom of an infection and/or to enhance the efficacy of another dose of a VLP or a vaccine. An effective dose may refer to the amount of VLPs or vaccines sufficient to delay or minimize the onset of an infection or a symptom of an infection. An effective dose may also refer to the amount of VLPs or vaccines that provides a therapeutic benefit in the treatment or management of an infection a symptom of an infection. Further, an effective dose is the amount with respect to VLPs or vaccines of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, (e.g., by plaque neutralization, complement fixation, or enzyme-linked immunosorbent, microneutralization assay) and/or responses of virus-specific CD4+ T cells and CD8+ T cells, and/or responses of other immune cells. In the case of a vaccine, an “effective dose” is one that prevents or reduces disease and/or prevents or reduces the severity of symptoms.

As used herein, the term “effective amount” refers to an amount of VLPs or vaccines comprising VLPs necessary or sufficient to realize a desired biologic effect. An effective amount of the composition would be the amount that achieves a selected result, and such an amount could be determined as a matter of routine experimentation by a person skilled in the art. For example, an effective amount for preventing, treating and/or ameliorating an infection could be that amount necessary to cause activation of the immune system, resulting in the development of an antigen specific immune response upon exposure to VLPs or vaccines comprising VLPs of the invention. The term is also synonymous with “sufficient amount.”

An “immunological response” or “immunity” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein the term “protective immune response”, “protective response”, or “protective immunity” refers to an immune response mediated by antibodies against an infectious agent, which is exhibited by a vertebrate (e.g., a human), that prevents or ameliorates an infection or reduces at least one symptom thereof. VLPs of the invention can stimulate the production of antibodies that, for example, neutralize infectious agents, block infectious agents from entering cells, block replication of said infectious agents, and/or protect host cells from infection and destruction. The term can also refer to an immune response that is mediated by T cells, B cells, and/or other white blood cells against an infectious agent, exhibited by a vertebrate (e.g., a human), that prevents or ameliorates RSV infection or reduces at least one symptom thereof.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition (e.g. a VLP or a vaccine comprising a VLP) refers to an amount that is effective to achieve a desired therapeutic result. In some embodiments, a desired therapeutic result is the prevention of an RSV infection and/or a symptom thereof. In some embodiments, a desired therapeutic result is the treatment of an RSV infection and/or a symptom thereof. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST,

BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “engineered” or “recombinant” means a polynucleotide or polypeptide of semisynthetic, or synthetic origin that either does not occur in nature or is operably linked to another polynucleotide in an arrangement not found in nature.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In some embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

A “vector” refers to a recombinant DNA plasmid, bacteriophage, or virus that comprises a heterologous polynucleotide to be delivered to a target cell, either in vitro or in vivo. The heterologous polynucleotide may comprise a sequence of interest for purposes of prevention or therapy, and may optionally be in the form of an expression cassette. As used herein, a vector may be able to but does not need to be capable of replication in the ultimate target cell or subject. The term includes vectors for cloning as well as viral vectors.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Virus-Like Particles and Vaccines

Respiratory Syncytial Virus (RSV) is the leading cause of severe bronchiolitis in infants and young children and a high priority for vaccine development. Disclosed herein are virus-like particles (VLPs) and the use thereof for inducing immune responses to RSV infection or at least one symptom thereof in a subject. VLPs are used herein for vaccines since they are immunogenic and are safe to human. The present disclosure provides RSV VLPs with F and/or G using an RSV platform with M plus P or M plus M2-1, and uses thereof for inducing protective immune responses.

The respiratory syncytial virus (RSV), a member of the species orthopneumovirus of Orthopneumovirus genus, is a syncytial virus that causes respiratory tract infections. RSV has a single stranded negative sense RNA genome which is approximately 15.2 Kb long. RSV has been classified into two groups (group A and group B, or termed as “strain A and strain B” herein) on the basis of genetic and antigenic heterogeneity. The two major glycoprotein on the surface of the RSV virion are the attachment glycoprotein (G) and fusion protein (F). G is involved in attachment of virion to the host cells, and F cause the virion membrane to fuse with cell membrane. In addition, four of the viral genes code for intracellular proteins that are involved in genome transcription, replication, and particle budding, namely N (nucleoprotein), P (phosphoprotein), M (matrix protein), and L (“large” protein, containing the RNA polymerase catalytic motifs).

In some aspects, disclosed herein is a virus like particle (VLP) comprising a respiratory syncytial virus (RSV) M protein and an RSV M2-1 protein.

As used herein, the term “RSV Matrix” or “RSV M” protein refers to an RSV protein that, when expressed in a host cell, induces formation of VLPs. An example of an RSV M protein is represented by SEQ ID NO: 39 or SEQ ID NO: 41. The term also comprises any variants, derivatives and/or fragments of RSV M protein that, when expressed in a host cell, induces formation of VLPs. In some embodiments, the M polypeptide comprises the sequence set forth in SEQ ID NO: 39 or SEQ ID NO: 41, or sequence having at or greater than about 80%, about 85%, about 90%, about 95%, about 98%, or about 99% homology with SEQ ID NO: 39 or SEQ ID NO: 41, or a polypeptide comprising a portion of SEQ ID NO: 39 or SEQ ID NO: 41. In some embodiments, the M polypeptide comprises the sequence set forth in SEQ ID NO: 39. In some embodiments, the M polypeptide comprises the sequence set forth in SEQ ID NO: 41. The term also encompasses nucleotide sequences which encode for RSV M and/or any variants, derivatives and/or fragments thereof that when transfected (or infected) into a host cell will express RSV M protein and induce formation of VLPs. In some embodiments, In some embodiments, the nucleotide sequence encoding M polypeptide comprises the sequence set forth in SEQ ID NO: 40 or SEQ ID NO: 42, or sequence having at or greater than about 80%, about 85%, about 90%, about 95%, about 98%, or about 99% homology with SEQ ID NO: 40 or SEQ ID NO: 42, or a polynucleotide comprising a portion of SEQ ID NO: 40 or SEQ ID NO: 42.

As used herein, the term “RSV M2-1 protein” or “M2-1 protein” refers to a cofactor of the RSV viral RNA polymerase complex and functions as a transcriptional processivity and antitermination factor. An example of an RSV M2-1 protein is represented by SEQ ID NO: 43 or SEQ ID NO: 45. The term also comprises any variants, derivatives and/or fragments of RSV M2-1 protein that, when expressed in a host cell, induces formation of VLPs. In some embodiments, the M2-1 polypeptide comprises the sequence set forth in SEQ ID NO: 43 or SEQ ID NO: 45, or sequence having at or greater than about 80%, about 85%, about 90%, about 95%, about 98%, or about 99% homology with SEQ ID NO: 43 or SEQ ID NO: 45, or a polypeptide comprising a portion of SEQ ID NO: 43 or SEQ ID NO: 45. In some embodiments, the M2-1 polypeptide comprises the sequence set forth in SEQ ID NO: 43. In some embodiments, the M2-1 polypeptide comprises the sequence set forth in SEQ ID NO: 45. The term also encompasses nucleotide sequences which encode for RSV M2-1 and/or any variants, derivatives and/or fragments thereof that when transfected (or infected) into a host cell will express RSV M2-1 protein and induce formation of VLPs. In some embodiments, the nucleotide sequence encoding M2-1 polypeptide comprises the sequence set forth in SEQ ID NO: 44 or SEQ ID NO: 46, or sequence having at or greater than about 80%, about 85%, about 90%, about 95%, about 98%, or about 99% homology with SEQ ID NO: 44 or SEQ ID NO: 46, or a polynucleotide comprising a portion of SEQ ID NO: 44 or SEQ ID NO: 46.

In some embodiments, the VLP of any preceding aspect comprises one or more additional RSV proteins. In some embodiments, the VLP comprises an RSV F protein. As used herein, the terms “RSV F protein”, “F protein” refers to an RSV fusion protein. The RSV F protein directs penetration of RSV by fusion between the virion's envelope protein and the host cell plasma membrane. Later in infection, the F protein expressed on the cell surface can mediate fusion with neighboring cells to form syncytia. The F protein is a type I transmembrane surface protein that has a N-terminal cleaved signal peptide and a membrane anchor near the C-terminus. RSV F is synthesized as an inactive F0 precursor that assembles into a homotrimer and is activated by cleavage in the trans-Golgi complex by a cellular endoprotease to yield two disulfide-linked subunits. The N-terminus of the F1 subunit that is created by cleavage contains a hydrophobic domain (the fusion peptide) that inserts directly into the target membrane to initiate fusion. The F1 subunit also contains heptad repeats that associate during fusion, driving a conformational shift that brings the viral and cellular membranes into close proximity. Because the F protein is expressed on the surface of infected cells and is responsible for subsequent fusion with other cells leading to syncytia formation, antibodies or cellular immune responses to the F protein can neutralize virus and/or block entry of the virus into the cell or prevent syncytia formation.

Accordingly, in some embodiments, the RSV F protein of any preceding aspect is selected from a group consisting of a pre-fusion form of the RSV F protein, a post-fusion form of the RSV

F protein, and a carbonyl terminal portion of the RSV F protein. In some embodiments, the RSV F protein is the pre-fusion form of the RSV F protein. In some embodiments, the RSV F protein is the post-fusion form of the RSV F protein. In some embodiments, the RSV F protein is the carbonyl terminal portion of the RSV F protein. In some embodiments, the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37.

In some embodiments, the F polypeptide comprises the sequence set forth in SEQ ID NO: 23. In some embodiments, the F polypeptide comprises the sequence set forth in SEQ ID NO: 26. In some embodiments, the F polypeptide comprises the sequence set forth in SEQ ID NO: 29. In some embodiments, the F polypeptide comprises the sequence set forth in SEQ ID NO: 32. In some embodiments, the F polypeptide comprises the sequence set forth in SEQ ID NO: 34. In some embodiments, the F polypeptide comprises the sequence set forth in SEQ ID NO: 36. In some embodiments, the F polypeptide comprises the sequence set forth in SEQ ID NO: 37.

The term “RSV F protein” or “F” protein also encompasses nucleotide sequences which encode for an RSV F and/or any variants, derivatives and/or fragments thereof that when transfected (or infected) into a host cell will express an RSV F protein and induce formation of VLPs. In some embodiments, the RSV F polypeptide comprises the sequence set forth in SEQ ID NO: 23, SEQ ID NO: 26, SEQ ID NO: 29, SEQ ID NO: 32, SEQ ID NO: 34, SEQ ID NO: 36, or SEQ ID NO: 37, or sequence having at or greater than about 80%, about 85%, about 90%, about 95%, about 98%, or about 99% homology with the sequence set forth in SEQ ID NO: 23, SEQ ID NO: 26, SEQ ID NO: 29, SEQ ID NO: 32, SEQ ID NO: 34, SEQ ID NO: 36, or SEQ ID NO: 37, or a polypeptide comprising a portion of the sequence set forth in SEQ ID NO: 23, SEQ ID NO: 26, SEQ ID NO: 29, SEQ ID NO: 32, SEQ ID NO: 34, SEQ ID NO: 36, or SEQ ID NO: 37. In some embodiments, the nucleotide sequence encoding the RSV F polypeptide comprises the sequence set forth in SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 35, or SEQ ID NO: 38, or sequence having at or greater than about 80%, about 85%, about 90%, about 95%, about 98%, or about 99% homology with SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 35, or SEQ ID NO: 38, or a polynucleotide comprising a portion of SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 35, or SEQ ID NO: 38.

In some embodiments, the carbonyl terminal portion form of the RSV F polypeptide comprises the sequence set forth in SEQ ID NO: 32, or sequence having at or greater than about 80%, about 85%, about 90%, about 95%, about 98%, or about 99% homology with SEQ ID NO: 32, or a polypeptide comprising a portion of SEQ ID NO: 32. The term also encompasses nucleotide sequences which encode for the carbonyl terminal portion form of the RSV F polypeptide and/or any variants, derivatives and/or fragments thereof that when transfected (or infected) into a host cell will express the carbonyl terminal portion form of the RSV F polypeptide and induce formation of VLPs. In some embodiments, the nucleotide sequence encoding the carbonyl terminal portion form of the RSV F polypeptide comprises the sequence set forth in SEQ ID NO: 33, or sequence having at or greater than about 80%, about 85%, about 90%, about 95%, about 98%, or about 99% homology with SEQ ID NO: 33, or a polynucleotide comprising a portion of SEQ ID NO: 33.

In some embodiments, the VLP of any preceding aspect further comprises an RSV G protein. In some embodiments, the RSV G protein is from RSV group A or RSV group B. In some embodiments, the RSV G protein is from RSV group A. In some embodiments, the RSV G protein is from RSV group B. In some embodiments, the RSV G protein is from RSV group A and RSV group B.

As used herein, the terms “RSV G protein” or “G protein” refers to a type II transmembrane glycoprotein with a single hydrophobic region near the N-terminal end that serves as both an uncleaved signal peptide and a membrane anchor, leaving the C-terminal two-thirds of the molecule oriented externally. In some embodiments, the RSV G polypeptide comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or sequence having at or greater than about 80%, about 85%, about 90%, about 95%, about 98%, or about 99% homology with a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or a polypeptide comprising a portion of a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21. The term also encompasses nucleotide sequences which encode for the RSV G polypeptide and/or any variants, derivatives and/or fragments thereof that when transfected (or infected) into a host cell will express various forms of the RSV G protein and induce formation of VLPs (for example, secreted or membrane-bound G proteins). In some embodiments, the nucleotide sequence encoding the RSV G protein comprises a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or sequence having at or greater than about 80%, about 85%, about 90%, about 95%, about 98%, or about 99% homology with a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or a polynucleotide comprising a portion of a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22. In some embodiments, the G protein comprises a sequence of SEQ ID NO: 1. In some embodiments, the G protein comprises a sequence of SEQ ID NO: 11. In some embodiments, the G protein comprises a sequence of SEQ ID NO: 15. In some embodiments, the G protein comprises a sequence of SEQ ID NO: 17.

The G protein structure consists of a central conserved region (CCD-G) that contains a CX3C chemokine motif that enables binding to the CX3C chemokine receptor, CX3CR1 (HGNC: 2558, Entrez Gene: 1524, Ensembl: ENSG00000168329, OMIM: 601470, UniProtKB: P49238), a crucial chemokine receptor involved in migration and adhesion of leukocytes. The CX3C motif is known to those of skill in the art. In some embodiments, the CX3C motif is from amino acid 182 to 186 of an RSV G protein from group A. G protein induced inflammatory responses can be reduce by blocking G protein binding to CX3CR1. The present disclosure shows that the anti-G protein antibodies induced by the vaccine disclosed herein and reduces RSV infection related inflammation. Therefore, in some embodiments, the VLPs of any preceding aspect comprises a recombinant RSV G protein, wherein the recombinant RSV G protein comprises an intracellular and transmembrane domain plus about 20 to about 25 aa of the extracellular domain and/or a CCD-G of an RSV G protein, wherein the RSV G protein is from RSV group A and/or group B. In some embodiments, the transmembrane domain term used herein can indicate intracellular and transmembrane domains plus about 20 to about 25 aa of the extracellular domain (e.g. aa 1-86 or 1-91). In some embodiments, the intracellular plus transmembrane plus initial sequences of the extracellular domain of an RSV G protein comprises amino acids 1-86 of an RSV from group A or amino acids 1-77 of an RSV from group B. In some embodiments, the CCD-G domain of an RSV G protein comprises amino acid 155-206 of an RSV G protein of group A or B or amino acid 146-197 of an RSV G protein of group A or B or 146-206 of group A or B. In some embodiments, the RSV G protein of group A comprises a sequence of SEQ ID NO: 7 or SEQ ID NO: 9. In some embodiments, the RSV G protein of group B comprises a sequence of SEQ ID NO: SEQ ID NO: 19 or SEQ ID NO: 21. The CX3C motif can be mutated for preventing the G protein in the VLP from binding to CX3CR1, such that the VLP can be more immunogenic and safer. Therefore, in some embodiments, the CCD-G comprises a mutated CX3C motif that has one or more amino acids inserted between the two cysteines of the CX3C motif. The one or more amino acids can be any amino acid. In some embodiments, the one or more amino acids are alanines. Accordingly, in some embodiments, the recombinant G protein of any preceding aspect further comprises mutated CX3C motif, wherein the recombinant G protein comprises a sequence selected from the group consisting of SEQ ID NO: 11 and SEQ ID NO: 13.

As noted above, the term “engineered” or “recombinant” means a polynucleotide or polypeptide of semisynthetic, or synthetic origin that either does not occur in nature or is operably linked to another polynucleotide in an arrangement not found in nature. In some embodiments, recombinant RSV G protein is a G protein comprising one or more transmembrane domains of RSV G proteins and one or more CCD-G domains of RSV G proteins, wherein the transmembrane domain and the CCD-G domain can be from a same RSV group or different RSV groups. In some embodiments, the recombinant RSV G protein comprises a transmembrane domain of an RSV G protein and a CCD-G domain of an RSV G protein, wherein the transmembrane domain and the CCD-G domain can be from a same RSV group or different RSV groups. Thus, in some embodiments, the VLP disclosed herein comprises a recombinant RSV G protein comprising a transmembrane domain of an RSV G protein of RSV group A and a CCD-G domain of an RSV G protein of RSV group A. In some embodiments, the VLP disclosed herein comprises a recombinant

RSV G protein comprising a transmembrane domain of an RSV G protein of RSV group A and a CCD-G domain of an RSV G protein of RSV group B. In some embodiments, the VLP disclosed herein comprises a recombinant RSV G protein comprising a transmembrane domain of an RSV G protein of RSV group B and a CCD-G domain of an RSV G protein of RSV group B. In some embodiments, the recombinant RSV G protein comprises a transmembrane domain of an RSV G protein and a group A CCD-G domain and a group B CCD-G domain of RSV G protein, wherein the transmembrane domain can be from either a group A or B RSV groups. In some embodiments, the VLP can have two recombinant G proteins, one for group A and one for group B. The recombinant RSV G protein of any preceding aspect, the transmembrane domain is operably linked to the CCD-G domain and the VLP also contains a form of the F protein.

Accordingly, in some embodiments, the recombinant G protein of any preceding aspect comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21. In some embodiments, the recombinant G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or sequence having at or greater than about 80%, about 85%, about 90%, about 95%, about 98%, or about 99% homology with a sequence selected from the group consisting of SEQ ID NO: 1, SEQ ID NO: 3, SEQ ID NO: 5,
SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or a polypeptide comprising a portion of a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21. In some embodiments, the nucleotide sequence encoding the recombinant G protein comprises a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or sequence having at or greater than about 80%, about 85%, about 90%, about 95%, about 98%, or about 99% homology with a sequence selected from the group consisting of SEQ ID NO: 2, SEQ ID NO: 4,
SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or a polynucleotide comprising a portion of a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22.

In some embodiments, the recombinant G protein comprises a sequence of SEQ ID NO: 1. In some embodiments, the recombinant G protein comprises a sequence of SEQ ID NO: 3. In some embodiments, the recombinant G protein comprises a sequence of SEQ ID NO: 5. In some embodiments, the recombinant G protein comprises a sequence of SEQ ID NO: 7. In some embodiments, the recombinant G protein comprises a sequence of SEQ ID NO: 9. In some embodiments, the recombinant G protein comprises a sequence of SEQ ID NO: 11. In some embodiments, the recombinant G protein comprises a sequence of SEQ ID NO: 13. In some embodiments, the recombinant G protein comprises a sequence of SEQ ID NO: 15. In some embodiments, the recombinant G protein comprises a sequence of SEQ ID NO: 17. In some embodiments, the recombinant G protein comprises a sequence of SEQ ID NO: 19. In some embodiments, the recombinant G protein comprises a sequence of SEQ ID NO: 21.

In some embodiments, the VLP of any preceding aspect further comprises an RSV P protein, an RSV N protein, or an RSV L protein.

In some aspects, disclosed herein is a virus like particle (VLP) comprising a respiratory syncytial virus (RSV) M protein, an RSV P protein, an RSV F protein, and an RSV G protein.

In some embodiments, the RSV F protein is selected from a group consisting of a pre-fusion form of the RSV F protein, a post-fusion form of the RSV F protein, and a carbonyl terminal portion of the RSV F protein. In some embodiments, the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37. In some embodiments, the carbonyl terminal portion of the RSV F protein comprises a sequence of SEQ ID NO: 32.

In some embodiments, the VLP comprises an RSV G protein. In some embodiments, the RSV G protein is from RSV group A or RSV group B. In some embodiments, the RSV G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21.

In some embodiments, the VLP comprises a recombinant RSV G protein. In some embodiments, the recombinant RSV G protein comprises a transmembrane domain of the RSV G protein and a central conserved domain of the RSV G protein.

As used herein, the term “RSVP protein” or “P protein” refers to an RSV phosphorylation. The colocalization of M2-1 protein with N protein and P protein as part of the ribonucleoprotein (RNP) complex contributes RSV viral gene transcription and replication. An example of an RSV P protein is represented by SEQ ID NO: 47 or SEQ ID NO: 49. The term also comprises any variants, derivatives and/or fragments of RSV P protein that, when expressed in a host cell, induces formation of VLPs. In some embodiments, the P polypeptide comprises the sequence set forth in

SEQ ID NO: 47 or SEQ ID NO: 49, or sequence having at or greater than about 80%, about 85%, about 90%, about 95%, about 98%, or about 99% homology with SEQ ID NO: 47 or SEQ ID NO: 49, or a polypeptide comprising a portion of SEQ ID NO: 47 or SEQ ID NO: 49. The term also encompasses nucleotide sequences which encode for RSVP and/or any variants, derivatives and/or fragments thereof that when transfected (or infected) into a host cell will express RSV P protein and induce formation of VLPs. In some embodiments, the nucleotide sequence encoding RSV P protein comprises the sequence set forth in SEQ ID NO: 48 or SEQ ID NO: 50, or sequence having at or greater than about 80%, about 85%, about 90%, about 95%, about 98%, or about 99% homology with SEQ ID NO: 48 or SEQ ID NO: 50, or a polypeptide comprising a portion of SEQ ID NO: 48 or SEQ ID NO: 50.

In some embodiments, disclosed herein is a virus like particle (VLP) comprising a respiratory syncytial virus (RSV) M protein, an RSV P protein, an RSV F protein, and an RSV G protein, wherein the VLP further comprises an RSV M2-1 protein, an RSV N protein, or an RSV L protein. Additional examples of RSV sequences and proteins are further described in U.S. Patent Application Publication U.S. 2008/0233150, which is incorporated herein by reference for all purposes.

In some aspects, disclosed herein are vaccines comprising VLPs of any preceding aspect. In some embodiments, the vaccine further comprises an adjuvant.

Optionally, the vaccine contemplated herein can be combined with an adjuvant such as Freund's incomplete adjuvant, Freund's Complete adjuvant, alum, monophosphoryl lipid A, alum phosphate or hydroxide, QS-21, salts, i.e., AlK(SO4)2, AlNa(SO4)2, AlNH4(SO4)2, silica, kaolin, carbon polynucleotides, i.e., poly IC and poly AU. Additional adjuvants can include QuilA and Alhydrogel and the like. Optionally, the vaccine contemplated herein can be combined with immunomodulators and immunostimulants such as interleukins, interferons and the like. Many vaccine formulations are known to those of skill in the art.

5 In some embodiments, the vaccine further comprises a pharmaceutically acceptable carrier.

### Methods of Use

In some aspects, disclosed herein is a method of inducing immunity to RSV infection or at least one symptom thereof in a subject, comprising administering one or more effective doses of a vaccine comprising a virus like particle (VLP), wherein the VLP comprises a respiratory syncytial virus (RSV) M protein and an RSV M2-1 protein.

In some embodiments, the VLP further comprises an F protein. In some embodiments, the VLP further comprises a G protein. In some embodiments, the VLP further comprises an F protein and a G protein.

In some aspects, disclosed herein is a method of inducing immunity to RSV infection or at least one symptom thereof in a subject, comprising administering one or more effective doses of a vaccine comprising a virus like particle (VLP), wherein the VLP comprises a respiratory syncytial virus (RSV) M protein, an RSV P protein, an RSV F protein, and an RSV G protein.

In some aspects, disclosed herein is a method of preventing an RSV infection or at least one symptom thereof in a subject, comprising administering one or more effective doses of a vaccine comprising a virus like particle (VLP), wherein the VLP comprises a respiratory syncytial virus (RSV) M protein and an RSV M2-1 protein. In some embodiments, the VLP further comprises an F protein. In some embodiments, the VLP further comprises a G protein. In some embodiments, the VLP further comprises an F protein and a G protein.

In some aspects, disclosed herein is a method of preventing an RSV infection or at least one symptom thereof in a subject, comprising administering one or more effective doses of a vaccine comprising a virus like particle (VLP), wherein the VLP comprises a respiratory syncytial virus (RSV) M protein, an RSV P protein, an RSV F protein, and an RSV G protein.

As used herein, the terms “RSV Matrix” or “RSV M” protein refer to an RSV protein that, when expressed in a host cell, induces formation of VLPs. An example of an RSV M protein is represented by SEQ ID NO: 39 or SEQ ID NO: 41. The term also comprises any variants, derivatives and/or fragments of RSV M protein that, when expressed in a host cell, induces formation of VLPs. In some embodiments, the M polypeptide comprises the sequence set forth in SEQ ID NO: 39 or SEQ ID NO: 41, or sequence having at or greater than about 80%, about 85%, about 90%, about 95%, about 98%, or about 99% homology with SEQ ID NO: 39 or SEQ ID NO: 41, or a polypeptide comprising a portion of SEQ ID NO: 39 or SEQ ID NO: 41. The term also encompasses nucleotide sequences which encode for RSV M and/or any variants, derivatives and/or fragments thereof that when transfected (or infected) into a host cell will express RSV M protein and induce formation of VLPs. In some embodiments, In some embodiments, the nucleotide sequence encoding M polypeptide comprises the sequence set forth in SEQ ID NO: 40 or SEQ ID NO: 42, or sequence having at or greater than about 80%, about 85%, about 90%, about 95%, about 98%, or about 99% homology with SEQ ID NO: 40 or SEQ ID NO: 42, or a polynucleotide comprising a portion of SEQ ID NO: 40 or SEQ ID NO: 42.

As used herein, the terms “RSV M2-1 protein”, “M2-1 protein” refers to a cofactor of the RSV viral RNA polymerase complex and functions as a transcriptional processivity and antitermination factor. An example of an RSV M2-1 protein is represented by SEQ ID NO: 43 or SEQ ID NO: 45. The term also comprises any variants, derivatives and/or fragments of RSV M2-1 protein that, when expressed in a host cell, induces formation of VLPs. In some embodiments, the M2-1 polypeptide comprises the sequence set forth in SEQ ID NO: 43 or SEQ ID NO: 45, or sequence having at or greater than about 80%, about 85%, about 90%, about 95%, about 98%, or about 99% homology with SEQ ID NO: 43 or SEQ ID NO: 45, or a polypeptide comprising a portion of SEQ ID NO: 43 or SEQ ID NO: 45. The term also encompasses nucleotide sequences which encode for RSV M2-1 and/or any variants, derivatives and/or fragments thereof that when transfected (or infected) into a host cell will express RSV M2-1 protein and induce formation of VLPs. In some embodiments, the nucleotide sequence encoding M2-1 polypeptide comprises the sequence set forth in SEQ ID NO: 44 or SEQ ID NO: 46, or sequence having at or greater than about 80%, about 85%, about 90%, about 95%, about 98%, or about 99% homology with SEQ ID NO: 44 or SEQ ID NO: 46, or a polynucleotide comprising a portion of SEQ ID NO: 44 or SEQ ID NO: 46.

In some embodiments, the VLP of any preceding aspect comprises one or more additional RSV proteins. In some embodiments, the VLP comprises an RSV F protein. As used herein, the terms “RSV F protein”, “F protein” refers to an RSV fusion protein.

Accordingly, in some embodiments, the RSV F protein of any preceding aspect is selected from a group consisting of a pre-fusion form of the RSV F protein, a post-fusion form of the RSV F protein, and a carbonyl terminal portion of the RSV F protein. In some embodiments, the RSV F protein is the pre-fusion form of the RSV F protein. In some embodiments, the RSV F protein is the post-fusion form of the RSV F protein. In some embodiments, the RSV F protein is the carbonyl terminal portion of the RSV F protein. Accordingly, in some embodiments, the RSV F protein of any preceding aspect is selected from a group consisting of a pre-fusion form of the RSV F protein, a post-fusion form of the RSV F protein, and a carbonyl terminal portion of the RSV F protein. In some embodiments, the RSV F protein is the pre-fusion form of the RSV F protein. In some embodiments, the RSV F protein is the post-fusion form of the RSV F protein. In some embodiments, the RSV F protein is the carbonyl terminal portion of the RSV F protein. In some embodiments, the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37.

The term “RSV F protein” or “F” protein also encompasses nucleotide sequences which encode for an RSV F and/or any variants, derivatives and/or fragments thereof that when transfected (or infected) into a host cell will express an RSV F protein and induce formation of VLPs. In some embodiments, the RSV F polypeptide comprises the sequence set forth in SEQ ID NO: 23, SEQ ID NO: 26, SEQ ID NO: 29, SEQ ID NO: 32, SEQ ID NO: 34, SEQ ID NO: 36, or SEQ ID NO: 37, or sequence having at or greater than about 80%, about 85%, about 90%, about 95%, about 98%, or about 99% homology with the sequence set forth in SEQ ID NO: 23, SEQ ID NO: 26, SEQ ID NO: 29, SEQ ID NO: 32, SEQ ID NO: 34, SEQ ID NO: 36, or SEQ ID NO: 37, or a polypeptide comprising a portion of the sequence set forth in SEQ ID NO: 23, SEQ ID NO: 26, SEQ ID NO: 29, SEQ ID NO: 32, SEQ ID NO: 34, SEQ ID NO: 36, or SEQ ID NO: 37. In some embodiments, the nucleotide sequence encoding the RSV F polypeptide comprises the sequence set forth in SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 35, or SEQ ID NO: 38, or sequence having at or greater than about 80%, about 85%, about 90%, about 95%, about 98%, or about 99% homology with SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 35, or SEQ ID NO: 38, or a polynucleotide comprising a portion of SEQ ID NO: 24, SEQ ID NO: 25, SEQ ID NO: 27, SEQ ID NO: 28, SEQ ID NO: 30, SEQ ID NO: 31, SEQ ID NO: 33, SEQ ID NO: 35, or SEQ ID NO: 38.

In some embodiments, the carbonyl terminal portion form of the RSV F polypeptide comprises the sequence set forth in SEQ ID NO: 32, or sequence having at or greater than about 80%, about 85%, about 90%, about 95%, about 98%, or about 99% homology with SEQ ID NO: 32, or a polypeptide comprising a portion of SEQ ID NO: 32. The term also encompasses nucleotide sequences which encode for the carbonyl terminal portion form of the RSV F polypeptide and/or any variants, derivatives and/or fragments thereof that when transfected (or infected) into a host cell will express the carbonyl terminal portion form of the RSV F polypeptide and induce formation of VLPs. In some embodiments, the nucleotide sequence encoding the carbonyl terminal portion form of the RSV F polypeptide comprises the sequence set forth in SEQ ID NO: 33, or sequence having at or greater than about 80%, about 85%, about 90%, about 95%, about 98%, or about 99% homology with SEQ ID NO: 33, or a polynucleotide comprising a portion of SEQ ID NO: 33.

In some embodiments, the VLP of any preceding aspect further comprises an RSV G protein. In some embodiments, the RSV G protein is from RSV group A or RSV group B. In some embodiments, the RSV G protein is from RSV group A. In some embodiments, the RSV G protein is from RSV group B. In some embodiments, the RSV G protein is from RSV group A and RSV group B.

As used herein, the terms “RSV G protein” or “G protein” refers to a type II transmembrane glycoprotein with a single hydrophobic region near the N-terminal end that serves as both an uncleaved signal peptide and a membrane anchor, leaving the C-terminal two-thirds of the molecule oriented externally. In some embodiments, the RSV G polypeptide comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or sequence having at or greater than about 80%, about 85%, about 90%, about 95%, about 98%, or about 99% homology with a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or a polypeptide comprising a portion of a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21. The term also encompasses nucleotide sequences which encode for the RSV G polypeptide and/or any variants, derivatives and/or fragments thereof that when transfected (or infected) into a host cell will express the post-fusion form of the RSV G and induce formation of VLPs. In some embodiments, the nucleotide sequence encoding the RSV G protein comprises a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or sequence having at or greater than about 80%, about 85%, about 90%, about 95%, about 98%, or about 99% homology with a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or a polynucleotide comprising a portion of a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22. In some embodiments, the G protein comprises a sequence of SEQ ID NO: 1. In some embodiments, the G protein comprises a sequence of SEQ ID NO: 11. In some embodiments, the G protein comprises a sequence of SEQ ID NO: 15. In some embodiments, the G protein comprises a sequence of SEQ ID NO: 17.

The G protein structure consists of a central conserved region (CCD-G) that contains a CX3C chemokine motif that enables binding to the CX3C chemokine receptor, CX3CR1 (HGNC: 2558, Entrez Gene: 1524, Ensembl: ENSG00000168329, OMIM: 601470, UniProtKB: P49238), a crucial chemokine receptor involved in migration and adhesion of leukocytes. G protein induced inflammatory responses can be reduce by blocking G protein binding to CX3CR1. The present disclosure shows that the anti-G protein antibodies induced by the vaccine disclosed herein reduce RSV infection related inflammation. Therefore, in some embodiments, the VLPs of any preceding aspect comprises a recombinant RSV G protein, wherein the recombinant RSV G protein comprises a transmembrane domain of an RSV G protein and/or a CCD-G of an RSV G protein, wherein the RSV G protein is from RSV group A and/or group B. In some embodiments, the transmembrane domain of an RSV G protein comprises amino acid 1-86 of an RSV from group A, amino acid 1-86 of an RSV from group B, or amino acid 1-77 of an RSV from group B. In some embodiments, the CCD-G domain of an RSV G protein comprises amino acid 155-206 of an RSV G protein of group A, amino acid 155-206 of an RSV G protein of group B, or amino acid 146-197 of an RSV G protein of group B. In some embodiments, the RSV G protein of group A comprises a sequence of SEQ ID NO: 7 or SEQ ID NO: 9. In some embodiments, the RSV G protein of group B comprises a sequence of SEQ ID NO: SEQ ID NO: 19 or SEQ ID NO: 21.

In some embodiments, recombinant RSV G protein is a G protein comprising one or more transmembrane domains of RSV G proteins and one or more CCD-G domains of RSV G proteins, wherein the transmembrane domain and the CCD-G domain can be from a same RSV group or different RSV groups. In some embodiments, the recombinant RSV G protein comprises a transmembrane domain of an RSV G protein and a CCD-G domain of an RSV G protein, wherein the transmembrane domain and the CCD-G domain can be from a same RSV group or different RSV groups. Thus, in some embodiments, the VLP disclosed herein comprises a recombinant RSV G protein comprising a transmembrane domain of an RSV G protein of RSV group A and a CCD-G domain of an RSV G protein of RSV group A. In some embodiments, the VLP disclosed herein comprises a recombinant RSV G protein comprising a transmembrane domain of an RSV G protein of RSV group A and a CCD-G domain of an RSV G protein of RSV group B. In some embodiments, the VLP disclosed herein comprises a recombinant RSV G protein comprising a transmembrane domain of an RSV G protein of RSV group B and a CCD-G domain of an RSV G protein of RSV group B. In some embodiments, the recombinant RSV G protein comprises more than one transmembrane domain of an RSV G protein and more than one CCD-G domain of an

RSV G protein, wherein the transmembrane domain and the CCD-G domain can be from a same RSV group or different RSV groups, wherein the RSV group can be RSV group A and/or group B. The recombinant RSV G protein of any preceding aspect, the transmembrane domain is operably linked to the CCD-G domain.

Accordingly, in some embodiments, the recombinant G protein of any preceding aspect comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21. In some embodiments, the recombinant G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or sequence having at or greater than about 80%, about 85%, about 90%, about 95%, about 98%, or about 99% homology with a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21, or a polypeptide comprising a portion of a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21. In some embodiments, the nucleotide sequence encoding the recombinant G protein comprises a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or sequence having at or greater than about 80%, about 85%, about 90%, about 95%, about 98%, or about 99% homology with a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22, or a polynucleotide comprising a portion of a sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, and SEQ ID NO: 22. In some embodiments, the recombinant G protein comprises a sequence of SEQ ID NO: 1. In some embodiments, the recombinant G protein comprises a sequence of SEQ ID NO: 3. In some embodiments, the recombinant G protein comprises a sequence of SEQ ID NO: 5. In some embodiments, the recombinant G protein comprises a sequence of SEQ ID NO: 7. In some embodiments, the recombinant G protein comprises a sequence of SEQ ID NO: 9. In some embodiments, the recombinant G protein comprises a sequence of SEQ ID NO: 11. In some embodiments, the recombinant G protein comprises a sequence of SEQ ID NO: 13. In some embodiments, the recombinant G protein comprises a sequence of SEQ ID NO: 15. In some embodiments, the recombinant G protein comprises a sequence of SEQ ID NO: 17. In some embodiments, the recombinant G protein comprises a sequence of SEQ ID NO: 19. In some embodiments, the recombinant G protein comprises a sequence of SEQ ID NO: 21.

In some embodiments, the VLP of any preceding aspect further comprises an RSV P protein, an RSV N protein, or an RSV L protein.

In some aspects, disclosed herein is a virus like particle (VLP) comprising a respiratory syncytial virus (RSV) M protein, an RSV P protein, an RSV F protein, and an RSV G protein.

In some embodiments, the RSV F protein is selected from a group consisting of a pre-fusion form of the RSV F protein, a post-fusion form of the RSV F protein, and a carbonyl terminal portion of the RSV F protein. In some embodiments, the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37. In some embodiments, the carbonyl terminal portion of the RSV F protein comprises a sequence of SEQ ID NO: 32.

In some embodiments, the VLP comprises an RSV G protein. In some embodiments, the RSV G protein is from RSV group A or RSV group B. In some embodiments, the RSV G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21.

As used herein, the terms “RSV P protein”, “P protein” refers to an RSV phosphorylation. The colocalization of M2-1 protein with N protein and P protein as part of the ribonucleoprotein (RNP) complex contributes RSV viral gene transcription and replication. An example of an RSV P protein is represented by SEQ ID NO: 47 or SEQ ID NO: 49. The term also comprises any variants, derivatives and/or fragments of RSV P protein that, when expressed in a host cell, induces formation of VLPs. In some embodiments, the P polypeptide comprises the sequence set forth in

SEQ ID NO: 47 or SEQ ID NO: 49, or sequence having at or greater than about 80%, about 85%, about 90%, about 95%, about 98%, or about 99% homology with SEQ ID NO: 47 or SEQ ID NO: 49, or a polypeptide comprising a portion of SEQ ID NO: 47 or SEQ ID NO: 49. The term also encompasses nucleotide sequences which encode for RSVP and/or any variants, derivatives and/or fragments thereof that when transfected (or infected) into a host cell will express RSV P protein and induce formation of VLPs. In some embodiments, the nucleotide sequence encoding RSV P protein comprises the sequence set forth in SEQ ID NO: 48 or SEQ ID NO: 50, or sequence having at or greater than about 80%, about 85%, about 90%, about 95%, about 98%, or about 99% homology with SEQ ID NO: 48 or SEQ ID NO: 50, or a polypeptide comprising a portion of SEQ ID NO: 48 or SEQ ID NO: 50.

In some embodiments, disclosed herein is a virus like particle (VLP) comprising a respiratory syncytial virus (RSV) M protein, an RSV P protein, an RSV F protein, and an RSV G protein, wherein the VLP further comprises an RSV M2-1 protein, an RSV N protein, or an RSV L protein.

In some aspects, disclosed herein are vaccines comprising VLPs of any preceding aspect.

In some embodiments, the vaccine further comprises an adjuvant.

Optionally, the vaccine contemplated herein can be combined with an adjuvant such as Freund's incomplete adjuvant, Freund's Complete adjuvant, alum, monophosphoryl lipid A, alum phosphate or hydroxide, QS-21, salts, i.e., AlK(SO4) 2, AlNa(SO4) 2, AlNH4 (SO4) 2, silica, kaolin, carbon polynucleotides, i.e., poly IC and poly AU. Additional adjuvants can include QuilA and Alhydrogel and the like. Optionally, the vaccine contemplated herein can be combined with immunomodulators and immunostimulants such as interleukins, interferons and the like. Many vaccine formulations are known to those of skill in the art.

In some embodiments, the vaccine further comprises a pharmaceutically acceptable carrier. The vaccines of the present invention can be administered to the appropriate subject in any manner known in the art, e.g., orally intramuscularly, intravenously, sublingual mucosal, intraarterially, intrathecally, intradermally, intraperitoneally, intranasally, intrapulmonarily, intraocularly, intravaginally, intrarectally or subcutaneously. They can be introduced into the gastrointestinal tract or the respiratory tract, e.g., by inhalation of a solution or powder containing the conjugates. In some embodiments, the compositions can be administered via absorption via a skin patch. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained. In some embodiments, the one or more effective doses of the vaccine are administered to the subject via a route that is selected from the group consisting of an intramuscular route, a subcutaneous route, an intradermal route, an oral administration, a nasal administration, and inhalation.

A pharmaceutical composition (e.g., a vaccine) is administered in an amount sufficient to elicit production of antibodies and activation of CD4+ T cells and CD8+ T cells as part of an immunogenic response. Dosage for any given patient depends upon many factors, including the patient's size, general health, sex, body surface area, age, the particular compound to be administered, time and route of administration, and other drugs being administered concurrently. Determination of optimal dosage is well within the abilities of a pharmacologist of ordinary skill.

The vaccine compositions are administered to subjects which may become infected by a

Listeria described herein, including but not limited to dogs, cats, rabbits, rodents, horses, livestock (e.g., cattle, sheep, goats, and pigs), zoo animals, ungulates, primates, and humans. In some embodiments, the preferred subject is a human. In some embodiments, the subject is an infant or a child. In some embodiments, the human has an age less than 15 years old, 12 years old, 11 years old, 10 years old, 9 years old, 8 years old, 7 years old, 6 years old, 5 years old, 4 years old, 3 years old, 2 years old, or 1 year old.

### EXAMPLES

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Expression of G and F on RSV M Based Virus-Like Particles

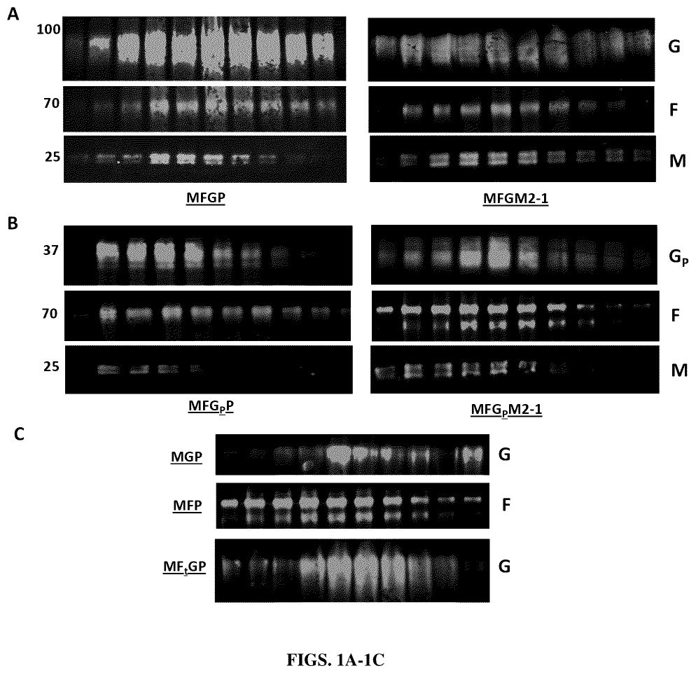

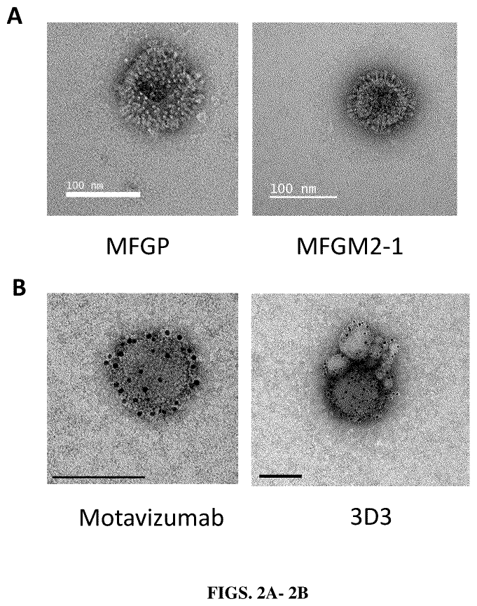

VLPs were generated from sequentially transfecting 293F cells with codon-optimized DNA plasmids containing RSV genes. The order of transfection is listed in Table 1. Western blot studies showed the successful development of VLPs with the M plus phosphoprotein P or the M plus M2-1 protein platforms with F or F and G (FIG. 1A), but only to very low levels with G alone, a condition not observed in VLPs expressing only F (FIG. 1C). The Western blot studies show detection of F or F and G in the supernatant and not the cell pellet of the induced 293F cells. Additionally, F or F and G were detected by Western blot in the pellet after centrifugation through a sucrose cushion indicating the proteins were particles and not dissolved in the media. Finally, F and M or F, G, and M were detected by Western blot in the same fractions after sucrose gradient purification indicating successful expression and purified of VLPs containing F or F and G (FIGS. 1A and 1B). The fact that G alone led to very low expression in VLPs and G with F gave high levels of VLPs with both indicated that F facilitated incorporation of G into VLPs. To further test this, a truncated F that consisted of the carbonyl terminus+transmembrane+26 amino acids of the amino terminus (Fₜ) was generated and this peptide was co-expressed with full-length G on M+P flatform. The data show that in the presence of Fₜ, G expression was abundant (FIG. 1C).

TABLE 1
Generation of RSV VLPs

| | Order of transfection | | | |
|---|---|---|---|---|
| VLPs | 1^{st} | 2^{nd} | 3^{rd} | 4^{th} |
| M | M | | | |
| MFP | M | P | F | |
| MFPG | M | P | F | G |
| MFPG_{P} | M | P | F | G_{P} |
| MFM2-1 | M | M2-1 | F | |
| MFM2-1G | M | M2-1 | F | G |
| MFM2-1G_{P} | M | M2-1 | F | G_{P} |

Since this platform allows incorporating different F and G constructs into the VLPs, further modification was done to make the F protein constructs most effective at inducing an anti-viral response and G protein constructs most effective at inducing anti-viral, anti-inflammatory responses. For example, RSV VLPs with M, M2-1, F and a truncated G (referred to as “G_{P}”; intra-cellular, transmembrane, and the 1^{st} 20 aa of the extracellular domain plus aa 155-206 of the G protein) were developed. This construct focuses on inducing antibodies against CCD-G, not other parts of G, and improves anti-CCD-G antibody titers. Because G's central conserved domain is crucial for the vaccines developed herein, the current study generated a G peptide that consisted of amino acids 1-86+155-206 (G_{P}) and generated full-length F and this G_{P} on the same VLPs utilizing either M+P or M+M2-1 flatform. Immunoblotting results from sucrose gradient show F, GP, and M bands from the same fractions, indicating that they were from the same particles (FIG. 1B).

RSV M protein based VLPs with M2-1+F, M2-1+F+G, and P+F+G have not been reported previously. The M2-1 protein has two advantages over the P protein; it is part of the structural proteins that stabilize the RSV virion structure, i.e. better models the natural virus, and it facilitates T-cells immune responses, making RSV M+M2-1 VLPs an ideal RSV vaccine. Importantly, with either the RSV M+P or M+M2-1 VLPs, the structure of G can be modified to focus the response on the part of G important for inducing anti-inflammatory and anti-viral immune responses, i.e. the central conserved domain of G (CCD-G). CCD-G includes amino acids 155-206 on the G protein. The sequences are highly conserved within but not between the two antigenic/genetic groups of RSV, A and B. In primary human airway epithelial cells and in human infection,

CX3CR1 is an important receptor for infection and anti-CCD-G antibodies have an added anti-viral effect in humans.

### Example 2. Electron Microscopic Study of VLPs

Electron microscopic studies of both MGFP and MFGM2-1 were performed to confirm the expression of G and F on VLPs. Negative stain showing spikes on VLP surface which were confirmed to include F spikes and/or G spikes by immunogold labeling with human anti-F motavizumab or human anti-G 3D3 monoclonal antibodies. Thus, the above data confirm successful generation of RSV matrix protein M+P or M+M2-1 based VLPs expressing both G and F.

### Example 3. Immunogenicity of VLPs

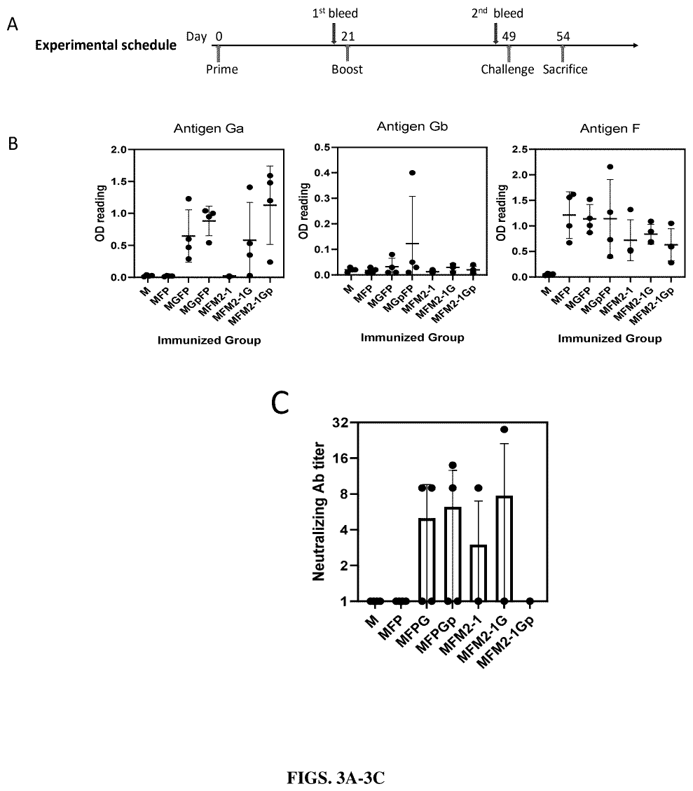

To determine the immunogenicity of the VLPs, BALB/c mice (n=4) were immunized with various VLPs as detailed in Table 2. All immunized animals were challenged with 106 TCID50 of RSV r19F at 4 weeks after the 2nd, booster, immunization (FIG. 3A). Blood specimens were collected before challenge and tested for F, Ga, and Gb binding antibodies by EIA and neutralizing antibodies by a micro-neutralization assay. All F and G VLP immunized animals and none of the control M VLP immunized animals developed F and Ga antibodies. Only one animal produced antibodies against Gb antigen (FIG. 3B). In addition, the short G peptide G_{P} was more efficient at inducing antibody than its full-length counterpart, indicating that the central conserved domain of G is an effective immunogen. Furthermore, VLP antigens that contained P induced antibodies against F better than those that contained M2-1 (FIG. 3B). In this study, the VLPs induced low levels of neutralizing antibodies. As shown, sera from animals in groups immunized with MFP and MFM2-1G_{P} did not possess neutralizing activity, but sera from other groups all had some neutralizing activity (FIG. 3C).

TABLE 2
Immunization schedule

| | | | | | RSV | |
|---|---|---|---|---|---|---|
| | | Dose | Immuni- | | challenge | |
| | | (per | zation | | TCID50/ | Days of |
| Group | N | mouse) | days | Route | mouse | harvest |
| M | 4 | 50 μg VLPs | 0, 21 | IM | 10⁶ at | 54 |
| | | | | | day 49 | |
| MFP | ″ | ″ | ″ | ″ | 10⁶ at | ″ |
| | | | | | day 49 | |
| MFGP | ″ | ″ | ″ | ″ | 10⁶ at | ″ |
| | | | | | day 49 | |
| MFG_{P}P | ″ | ″ | ″ | ″ | 10⁶ at | ″ |
| | | | | | day 49 | |
| MFM2-1 | ″ | ″ | ″ | ″ | 10⁶ at | ″ |
| | | | | | day 49 | |
| MFGM2-1 | ″ | ″ | ″ | ″ | 10⁶ at | ″ |
| | | | | | day 49 | |
| MFG_{P}M2-1 | ″ | ″ | ″ | ″ | 10⁶ at | ″ |
| | | | | | day 49 | |

### Example 4. Immunized Animals have Reduced Lung Viral Titers

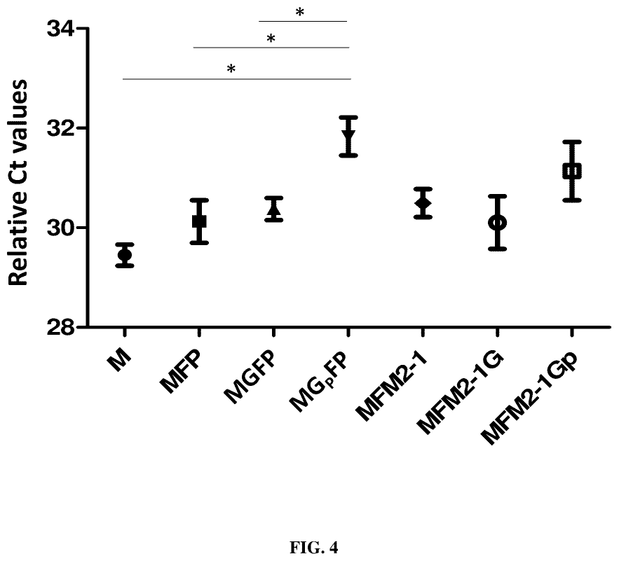

Next, the ability of the VLPs to prevent virus replication after challenge was investigated. The relative cycle threshold (CT) values were significant higher indicating less virus replication in animal groups that had P protein as part of the antigen VLPs, i.e. MFP, MFGP, and MFG_{P}P compared to the control antigen (M only VLPs). A higher value of CT correlates with low copy of the gene being evaluated. Also, there were significant differences between MFG_{P}P and MFP or MFGP (FIG. 4), indicating that anti-G antibodies participate in viral clearance in the lungs and that G_{P} was more effective in facilitating virus clearance than full length G. Moreover, VLP antigens that contained M2-1 protein, i.e. MFM2-1, MFGM2-1, and MFG_{P}M2-1 had higher CT values compared to control (FIG. 4).

### Example 5. Lung Histopathology

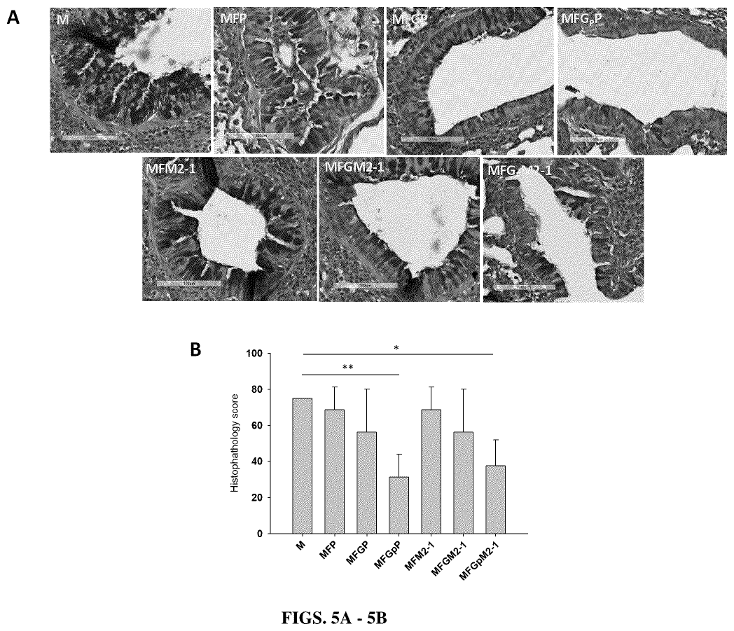

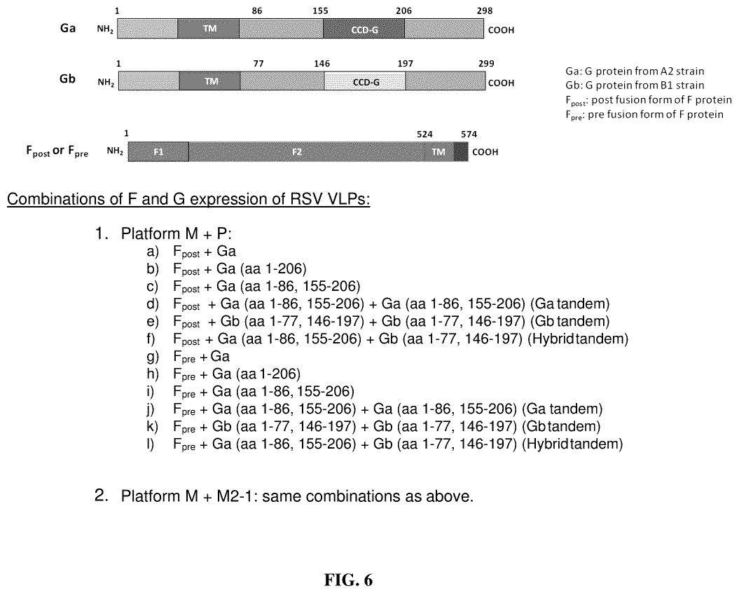

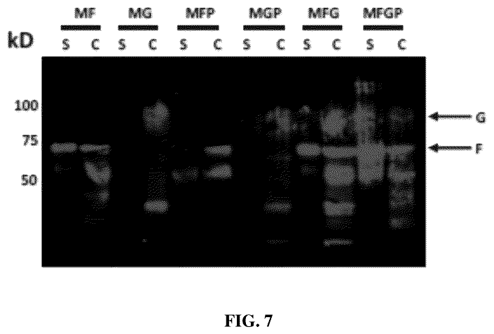

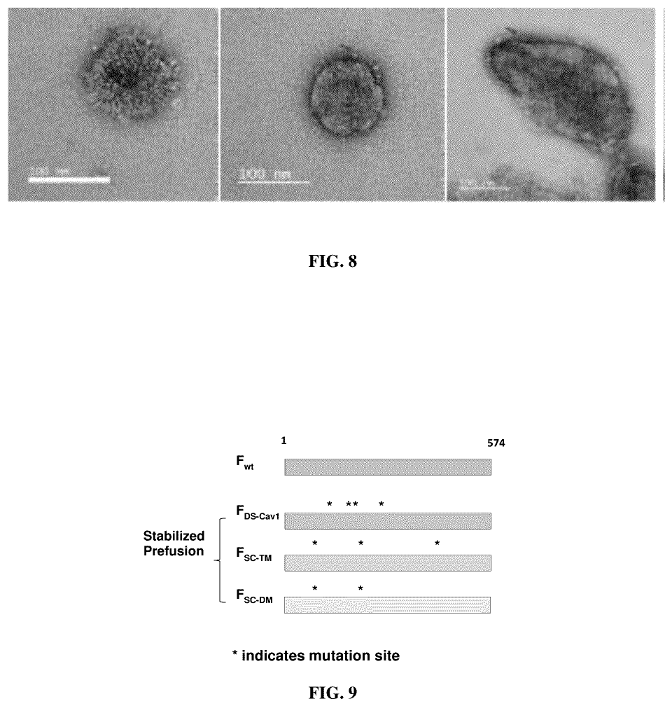

Since one of the manifestations of RSV infection with r19F virus is overproduction of mucin, pulmonary inflammation was examined in challenged animals by Periodic acid-Schiff staining (PAS). The stained slides were analyzed by Aperio ImageScope software (Leica, Germany) and scored blindly using 0-4 severity scale then converted to 0-100 histopathology scale. Positive PAS staining was found, indicating the presence of mucin, in the lungs of all immunized animals but less so than the control animals immunized with M only VLPs (FIGS. 5A and 5B). Only MFG_{P}P- and MFG_{P}M2-1-immunized animals had a significant reduction of PAS staining compared to control animals though (FIGS. 5A and 5B). These data show that F plus Gp VLPs were most effective at reducing both lung virus replication and mucin production.

### Example 6. Discussion

An RSV based VLP platform is developed herein to express F+G, F+G peptide, or G plus a truncated F. The G plus truncated F VLP makes it possible to study G without most of the extracellular F protein, and approximates the structure, function, and immunogenicity of G in an RSV VLP platform without F. The carboxy terminal intracellular plus transmembrane plus a part of the initial extracellular domain of F was needed to efficiently incorporate G into the VLPs. These data show that the RSV VLP platform has advantages over the platforms previously used in Newcastle disease or influenza virus or bacterial phage P22 platforms with the RSV F and/or G proteins.

The RSV platform VLPs better represent native structures of F and G, and facilitate studies of their structure and function relationships. It also provides an all RSV antigen platform to develop F and G VLP vaccines. A truncated F (Fₜ) that consists of the carbonyl terminus+transmembrane+26 amino acids of the amino terminus was generated and was co-expressed with a full-length G in M+P RSV VLP platform. This led to efficient G+Fₜ VLPs which can be used to study G with minimal interference by extracellular F. The data also determined that peptides of extracellular G can be incorporated as a mean to focus responses to certain regions of G. Studies show a critical role of the central conserved domain of G (CCD-G) in RSV disease pathogenesis and generating anti-CCD-G antibodies decreases disease through both an anti-viral and anti-inflammatory effect. CCD-G contains the CX3C motif through which G binds to CX3CR1 and antibodies that block binding to CX3CR1 decreases infection since CX3CR1 is an important receptor for infection of human airway epithelial cells. Studies in animals and in human cells in vitro show that blocking binding to CX3CR1 effects the inflammatory response to RSV. The present disclosure shows the successful generation of VLPs with a truncated G composed of amino acids 1-86+155-206 of G (G_{P}) with F in both M+P and M+M2-1 platforms.

P plays a crucial role in RSV polymerase activity interacting with both N and L protein in the process and also interacts with M2-1, but these roles do not suggest how it might help VLP formation when co-expressed with M. The M2-1 protein has transcription anti-termination activity and directly interacts with the M matrix protein providing a link to the RNA containing nucleocapsid.

Electron microscope negative stain studies revealed that both MFGP and MFGM2-1 VLPs expressed glycoproteins F and G as “spikes”. Immunoelectron microscopic studies confirmed the presence of F and G on VLP surfaces. No significant electron microscopic differences were observed between the two platforms of VLPs. The mouse studies show that the VLPs were immunogenic and induced serum antibodies against both F and G proteins at similar levels with either platform. Notably, the VLPs with Gp induced higher antibody titers and were more effective than VLPs with full length G. The M2-1 protein is effective at inducing short term protective T cell immunity, making it an ideal antigen for vaccine design. Of note, the G protein sequences used in this study are from a group A strain, A2 and these G sequences efficiently induced antibodies against a group A but not a group B G protein. Note that though CCD-G region, with the exception of a 13 amino acid sequence, is distinct between group A and group B strains, most of CCD-G is conserved within a group. Also, serum neutralizing antibody titers were tested, showing that immunized animals developed low levels of neutralizing antibodies. VLPs with a pre-fusion stabilized F protein can be more effective at inducing neutralizing antibodies.

Furthermore, animals immunized with G_{P} and F expressed in either M plus P or M plus M2-1 VLPs had reduced lung viral titer and lung inflammation compared to those immunized with the control VLPs, demonstrating that both VLP platforms were effective.

The current VLPs described herein can express F and/or G with two RSV platforms (M+P proteins or M+M2-1 proteins). With both platforms F and/or G can be modified to focus specific structural and antigenic features of the proteins. Given the failures in RSV vaccine development, combining the highly effective anti-viral activity of F-induced antibodies plus anti-inflammatory and immune-enhancing features of G provides an effective RSV vaccine.

### Example 7. Materials and Methods

Cells, Media and Plasmids: 293F cells stably transfected with plasmid pcDNA6/TR were provide by Dr. Xuemin Chen (Emory University) and cultured in freestyle 293 media (Gibco) on a shaker at 37° C., 8% CO₂. pcDNA3.1 DNA plasmids containing codon-optimized RSV genes M, M2-1, P, G from A2 strain, and F from A2 strain were provided by Dr. Marty Moore (now at Meissa Vaccines) were digested by KpnI and XhOI enzymes and cloned into KpnI and XhOI double digested pcDNA4/TO or pcDNA5/TO vector. Human codon optimized truncated Gof amino acids 1-86+155-206 and synthesized by Genescript (Piscataway, NJ). The gene provided in pUC57 plasmid was double digested by BamHI and XhOI enzymes and cloned into BamHI and XhOI double digested pcDNA5/TO vector. All genes were sequenced to confirm authenticity prior to transfection. To generate VLPs, 293F cells were sequentially and stably transfected with the RSV genes noted below.

Virus-like particles expression and purification: 20-30×10⁶ 293F cells were induced with 2 ug/ml of doxycycline for 72 h. Cells were centrifuged at 300×g for 10 min and the VLP-containing media was filtered through 0.45 μm filter followed by centrifugation through 20% sucrose cushion at 12,200×g for 2 h at 4° C. (SW Ti 32 rotor, Optima L-90K Ultracentrifuge, Beckman Coulter). The top layer of cell media and sucrose was thoroughly removed and the pellet was soaked in sterile PBS for 1 h on ice and resuspended. For sucrose gradient experiments, preparation of a linear sucrose gradient was described previously [Stone A B et al., 2009], 1 ml of the gradient was removed before the resuspended VLPs was layered onto the gradient and centrifuged with a Beckman Coulter SW 41 rotor at 11,000×g for 12 h at 4° C. A total of 10 1-ml fractions were removed from top, diluted 3× with sterile PBS, and centrifuged at 12,000×g for 1 h at 4° C. on a bench-top refrigerator. Supernatants were completely removed and pellets were soaked in sterile PBS for 1 h on ice before being resuspended.

Antibodies and Immunoblotting: the anti-G protein monoclonal antibody (mAb) 3D3 was provide by Trellis Bioscience (Redwood City, CA); the anti-F protein mAb motavizumab was provided by MedImmune (Gaithersburg, MD); rabbit serum anti-M antibody was provided by Dr. Oomens (Oklahoma State University); and goat anti RSV antibody was obtained from Millipore

(Burlington, MA). All anti-species fluorescent-conjugated secondary antibodies used in immunoblotting were obtained from LI-COR biosciences (Lincoln, NE). All HRP-conjugated secondary antibodies used in enzyme-linked immunosorbent assays (EIAs) were obtained from Jackson ImmunoResearch (West Grove, PA). For immunoblotting experiments, VLP samples were mixed with 2x Laemmli sample buffer (Bio-Rad) and boiled at 95° C. for 5 min. Samples were run on SDS-PAGE, transferred to a nitrocellulose membrane, blocked for 30 min in blocking buffer (5% dry milk in TTBS). After blotting with primary antibody (incubation period), membrane was washed 3x in TTBS following secondary antibody incubation (time) and 3x washes in TTBS. Signals were visualized by Odyssey CLX imaging system (LI-COR).

F, Ga, and Gb antibody EIAs: the secreted form of F, Ga or Gb protein antigens was produced from stably transfected 293F cells in serum-free media and coated onto 96-well microtiter plate in buffer. The plates were incubated in 2% nonfat dry milk dissolved in PBS blocking solution for 2 h at 37° C., washed with PBS-T, and serum specimens at 1:200 dilution added to the wells, incubated for 1 h at 37° C., the plates washed with PBS-T, and goat anti mouse IgG-HRP (1:5,000) added and incubated for 1 h at 37° C. Color was developed with OPD substrate and the reaction stopped after r 30 min at RT with 4N H₂SO₄. Optical density (OD) was measured at 490 nm and geometric mean of the OD490 was calculated from the triplicate wells.

RSV neutralizing antibody assay: heat inactivated sera were serially 2-fold diluted starting with a 1:10 dilution in MEM containing 0.5% FBS, incubated with RSV/A2 (100 TCID50) for 1 h at RT, and inoculated in triplicates onto non-confluent HEp-2 monolayers in 96-well plates for 1 h at 37° C. in a 5% CO₂ incubator. MEM containing 5% FBS was added to all the wells and cells were incubated for 3 days at 37° C. in a 5% CO₂ incubator. Cells were washed with PBS and fixed with 4% paraformaldehyde and RSV antigens detected by EIA with goat anti RSV antibody (1:5,000) followed with donkey anti goat IgG-HRP secondary antibody (1:5,000). Color was developed with OPD substrate and neutralization defined as a ≥50% reduction in OD value. The titer was estimated using the Reed and Much method. The geometric means+SEM for all animals in a group at any given time were calculated.

Virus: A recombinant virus of RSV A2 backbone expressing the F protein from L19 virus (r19F) [Moore M L et al., 2009] was chosen as the challenge virus since it induces airway disease that parallel RSV infection in humans but not seen with RSV A2. Stock virus was prepared by inoculating onto subconfluent HEp-2 at a multiplicity of infection (MOI) 0.01 for 2 h at 37° C. in 5% CO₂ incubator using 0.5% fetal bovine serum (FBS)-containing minimal essential medium (MEM). 5% FBS MEM was added and cells were incubated in 37° C., 5% CO₂ incubator for 3 days. Cells were frozen and thawed twice at −80C and 4° C., respectively, and centrifuged at 2,000 rpm for 15 min at 4° C. to remove cellular debris. The supernatant was layered onto 20% sucrose layer and centrifuged at 12,200×g for 2 h at 4° C. Pellet was resuspended in serum free MEM, divided into aliquots, and snap frozen in liquid nitrogen. The aliquots were stored at −80° C. until use. The infectivity titer of the inoculum was determined by serial 10-fold dilutions in subconfluent HEp-2 cell monolayer for 3 days and virus replication detected by EIA for RSV antigens with goat-anti RSV antibody. Titer was estimated from wells with absorbance >3standard deviations above the mean absorbance for wells without virus by Reed and Muench method.

Animal study: 4-6 weeks old female BALB/c mice were purchased from Charles River Lab (Wilmington, MA) and housed at Emory's Pediatric animal facility under food ad libitum in microisolator cages with auto sterilized water. All animal handlings and procedures were carried out according to protocol approved by Emory University (Atlanta, GA) Institutional Animals Care and Use Committee. For challenge study, mice were intranasally infected with 106 CTID50 RSV r19F A2 in 40 μl.

Real-time PCR: total RNA was extracted and purified from lung homogenates using Qiagen RNeasy kit (QIAGEN). RNA was reverse transcribed into cDNA using iScript™ cDNA synthesis kit (Bio-Rad) following the manufacturer's instruction. Quantitative PCR was carried out on a 7500 Fast Real-time PCR system (Applied Biosystems) using Power SYBR Green PCR master mix (Applied Biosystems). CT values were normalized using control β-actin CT values from the same samples. RSV matrix M gene primers and amplification cycles were described previously [Boyoglu-Barnum S et al., 2017]. Other primer pairs used were: B-actin, forward 5′-CAC CAA CTG GGA CGA CAT-3′, reverse 5′-ACA GCC TGG ATA GCA ACG-3′. mRNA levels were expressed as the geometric mean±SEM for all animals in a group.

Pulmonary histopathology: lungs were isolated and fixed in 10% neutral buffered formalin for 24 h. The lungs were then embedded in paraffin, sectioned, and stained with Periodic acid-Schiff (PAS). Slides were analyzed by Aperio ImageScope software (Leica) and scored blinded to treatment on a 0-4 scale and subsequently converted to a 0-100% histopathology scale.

Statistical Analysis: unless otherwise indicated, different groups were compared by Wilcoxon rank sum test or Wilcoxon matched pairs test. A p value of <0.05 was considered statistically significant. Data are shown as means and standard errors of the mean (SEM).

### REFERENCES

1. Hall C B, Simoes E A, Anderson L J. Clinical and epidemiologic features of respiratory syncytial virus. Curr Top Microbiol Immunol. 2013; 372:39-57.
2. Shi T, McAllister D A, O'Brien K L, et al. Global, regional, and national disease burden estimates of acute lower respiratory infections due to respiratory syncytial virus in young children in 2015: a systematic review and modelling study. Lancet. 2017; 390 (10098): 946-958.
3. Jartti T, Gern J E. Role of viral infections in the development and exacerbation of asthma in children. J Allergy Clin Immunol. 2017; 140 (4): 895-906.
4. Wu P, Hartert T V. Evidence for a causal relationship between respiratory syncytial virus infection and asthma. Expert Rev Anti Infect Ther. 2011; 9 (9): 731-745.
5. Stockman L J, Curns A T, Anderson L J, Fischer-Langley G. Respiratory Syncytial Virus-associated Hospitalizations Among Infants and Young Children in the United States, 1997-2006. Pediatr Infect Dis J. 2012; 31 (1): 5-9.
6. Hall C B, Weinberg G A, Iwane M K, et al. The burden of respiratory syncytial virus infection in young children. N Engl J Med. 2009; 360 (6): 588-598.
7. Mazur N I, Higgins D, Nunes M C, et al. The respiratory syncytial virus vaccine landscape: lessons from the graveyard and promising candidates. Lancet Infect Dis. 2018; 18 (10):e295-e311.
8. Collins P L, Fearns R, Graham B S. Respiratory Syncytial Virus: Virology, Reverse Genetics, and Pathogenesis of Disease. In: Anderson L J, Graham B S, eds. Challenges and Opportunities for Respiratory Syncytial Virus Vaccines. Current Topics in Microbiology and Immunology, vol 372. Vol 372. Berlin, Heidelberg Springer; 2013:3-38.
9. Anderson L J. Respiratory syncytial virus vaccine development. Seminars in immunology. 2013; 25 (2): 160-171.
10. Schepens B, Sedeyn K, Vande Ginste L, et al. Protection and mechanism of action of a novel human respiratory syncytial virus vaccine candidate based on the extracellular domain of small hydrophobic protein. EMBO Mol Med. 2014; 6 (11): 1436-1454.
11. Ngwuta J O, Chen M, Modjarrad K, et al. Prefusion F-specific antibodies determine the magnitude of RSV neutralizing activity in human sera. Science translational medicine. 2015; 7 (309): 309ra162.
12. Graham B S. Immunological goals for respiratory syncytial virus vaccine development. Curr Opin Immunol. 2019; 59:57-64.
13. Tripp R A, Power U F, Openshaw PJM, Kauvar L M. Respiratory Syncytial Virus: Targeting the G Protein Provides a New Approach for an Old Problem. J Virol. 2018; 92 (3).
14. Tripp R A. CX3C chemokine mimicry by respiratory syncytial virus G glycoprotein. In: Mahalingam S, ed. Chemokines in Viral Infection. Landes Bioscience; 2003.
15. Boyoglu-Barnum S, Gaston K A, Todd S O, et al. A respiratory syncytial virus (RSV) anti-G protein F(ab′)2 monoclonal antibody suppresses mucous production and breathing effort in RSV rA2-line19F-infected BALB/c mice. J Virol. 2013; 87 (20): 10955-10967.
16. Boyoglu-Barnum S, Todd S O, Chirkova T, et al. An anti-G protein monoclonal antibody treats RSV disease more effectively than an anti-F monoclonal antibody in BALB/c mice. Virology. 2015; 483:117-125.
17. Radu G U, Caidi H, Miao C, Tripp R A, Anderson L J, Haynes L M. Prophylactic treatment with a G glycoprotein monoclonal antibody reduces pulmonary inflammation in respiratory syncytial virus (RSV)-challenged naive and formalin-inactivated RSV-immunized BALB/c mice. J Virol. 2010; 84 (18): 9632-9636.
18. Rey G U, Miao C, Caidi H, et al. Decrease in formalin-inactivated respiratory syncytial virus (FI-RSV) enhanced disease with RSV G glycoprotein peptide immunization in BALB/c mice. PLOS One. 2013; 8 (12):e83075.
19. Jorquera P A, Choi Y, Oakley K E, et al. Nanoparticle vaccines encompassing the respiratory syncytial virus (RSV) G protein CX3C chemokine motif induce robust immunity protecting from challenge and disease. PLOS One. 2013; 8 (9):e74905.
20. Zhang W, Choi Y, Haynes L M, et al. Vaccination to induce antibodies blocking the CX3C-CX3CR1 interaction of respiratory syncytial virus G protein reduces pulmonary inflammation and virus replication in mice. J Virol. 2010; 84 (2): 1148-1157.
21. Murawski M R, McGinnes L W, Finberg R W, et al. Newcastle disease virus-like particles containing respiratory syncytial virus G protein induced protection in BALB/c mice, with no evidence of immunopathology. J Virol. 2010; 84 (2): 1110-1123.
22. Quan F S, Kim Y, Lee S, et al. Viruslike particle vaccine induces protection against respiratory syncytial virus infection in mice. J Infect Dis. 2011; 204 (7): 987-995.
23. Schwarz B, Morabito K M, Ruckwardt T J, et al. Viruslike Particles Encapsidating Respiratory Syncytial Virus M and M2 Proteins Induce Robust T Cell Responses. ACS Biomater Sci Eng. 2016; 2 (12): 2324-2332.

24 Meshram C D, Baviskar P S, Ognibene C M, Oomens AGP. The Respiratory Syncytial Virus Phosphoprotein, Matrix Protein, and Fusion Protein Carboxy-Terminal Domain Drive Efficient Filamentous Virus-Like Particle Formation. J Virol. 2016; 90 (23): 10612-10628.
25. Shaikh F Y, Cox R G, Lifland A W, et al. A critical phenylalanine residue in the respiratory syncytial virus fusion protein cytoplasmic tail mediates assembly of internal viral proteins into viral filaments and particles. mBio. 2012; 3 (1).
26. Walpita P, Johns L M, Tandon R, Moore M L. Mammalian Cell-Derived Respiratory Syncytial Virus-Like Particles Protect the Lower as well as the Upper Respiratory Tract. PLOS One. 2015; 10 (7):e0130755.
27. Stone A B. A simplified method for preparing sucrose gradients. Biochem J. 1974; 137 (1): 117-118.
28. Moore M L, Chi M H, Luongo C, et al. A chimeric A2 strain of respiratory syncytial virus (RSV) with the fusion protein of RSV strain line 19 exhibits enhanced viral load, mucus, and airway dysfunction. J Virol. 2009; 83 (9): 4185-4194.
29. Boyoglu-Barnum S, Todd S O, Meng J, et al. Mutating the CX3C Motif in the G Protein Should Make a Live Respiratory Syncytial Virus Vaccine Safer and More Effective. J Virol. 2017; 91 (10).
30. Liu J, Haddad E K, Marceau J, et al. A Numerically Subdominant CD8 T Cell Response to Matrix Protein of Respiratory Syncytial Virus Controls Infection with Limited Immunopathology. PLOS Pathog. 2016; 12 (3):e1005486.
31. Kiss G, Holl J M, Williams G M, et al. Structural analysis of respiratory syncytial virus reveals the position of M2-1 between the matrix protein and the ribonucleoprotein complex. J Virol. 2014; 88 (13): 7602-7617.
32. Li D, Jans D A, Bardin P G, Meanger J, Mills J, Ghildyal R. Association of respiratory syncytial virus M protein with viral nucleocapsids is mediated by the M2-1 protein. J Virol. 2008; 82 (17): 8863-8870.
33. Chirkova T, Lin S, Oomens A G, et al. CX3CR1 is an important surface molecule for respiratory syncytial virus infection in human airway epithelial cells. J Gen Virol. 2015; 96 (9): 2543-2556.
34. Jeong K I, Piepenhagen P A, Kishko M, et al. CX3CR1 Is Expressed in Differentiated Human Ciliated Airway Cells and Co-Localizes with Respiratory Syncytial Virus on Cilia in a G Protein-Dependent Manner. PLOS One. 2015; 10 (6):e0130517.
35. Johnson S M, McNally B A, Ioannidis I, et al. Respiratory Syncytial Virus Uses CX3CR1 as a Receptor on Primary Human Airway Epithelial Cultures. PLOS Pathog. 2015; 11 (12):e1005318.
36. Chirkova T, Boyoglu-Barnum S, Gaston K A, et al. Respiratory syncytial virus G protein CX3C motif impairs human airway epithelial and immune cell responses. J Virol. 2013; 87 (24): 13466-13479.
37. Meshram C D, Baviskar P S, Ognibene C M, Oomens A G. The Respiratory Syncytial Virus Phosphoprotein, Matrix Protein, and Fusion Protein Carboxy-Terminal Domain Drive Efficient Filamentous Virus-Like Particle Formation. J Virol. 2016; 90 (23): 10612-10628.
38. Cowton V M, McGivern D R, Fearns R. Unravelling the complexities of respiratory syncytial virus RNA synthesis. J Gen Virol. 2006; 87 (Pt 7): 1805-1821.
39. Bakker S E, Duquerroy S, Galloux M, et al. The respiratory syncytial virus nucleoprotein-RNA complex forms a left-handed helical nucleocapsid. J Gen Virol. 2013; 94 (Pt 8): 1734-1738.
40. Tawar R G, Duquerroy S, Vonrhein C, et al. Crystal structure of a nucleocapsid-like nucleoprotein-RNA complex of respiratory syncytial virus. Science. 2009; 326 (5957): 1279-1283.
41. Esperante S A, Paris G, de Prat-Gay G. Modular unfolding and dissociation of the human respiratory syncytial virus phosphoprotein p and its interaction with the m (2-1) antiterminator: a singular tetramer-tetramer interface arrangement. Biochemistry. 2012; 51 (41): 8100-8110.
42. Fearns R, Collins P L. Role of the M2-1 transcription antitermination protein of respiratory syncytial virus in sequential transcription. J Virol. 1999; 73 (7): 5852-5864.
43. Tanner S J, Ariza A, Richard C A, et al. Crystal structure of the essential transcription antiterminator M2-1 protein of human respiratory syncytial virus and implications of its phosphorylation. Proc Natl Acad Sci USA. 2014; 111 (4):1580-1585.
44. Ke Z, Dillard R S, Chirkova T, et al. The Morphology and Assembly of Respiratory Syncytial Virus Revealed by Cryo-Electron Tomography. Viruses. 2018; 10 (8).
45. Collins P L C J. Respiratory syncytial virus and metapneumovirus. In: Fields virology, 5th ed. Philadelphia, P A: Lippincott Williams & Wilkins; 2007:1601-1646.
46. Connors M, Collins P L, Firestone C-Y, Murphy B R. Respiratory syncytial virus (RSV) F, G, M2 (22K), and N proteins each induce resistance to RSV Challenge, but resistance induced by M2 and N proteins is relatively short-lived. J Virol. 1991; 65:1634-1637.
47. Green C A, Scarselli E, Sande C J, et al. Chimpanzee adenovirus- and MVA-vectored respiratory syncytial virus vaccine is safe and immunogenic in adults. Science translational medicine. 2015; 7 (300): 300ra126.
48. Mclellan J S, Ray W C, Peeples M E. Structure and function of respiratory syncytial virus surface glycoproteins. Curr Top Microbiol Immunol. 2013; 372:83-104.
49. McLellan J S, Chen M, Joyce M G, et al. Structure-based design of a fusion glycoprotein vaccine for respiratory syncytial virus. Science. 2013; 342 (6158): 592-598.
50. Krarup A, Truan D, Furmanova-Hollenstein P, et al. A highly stable prefusion RSV F vaccine derived from structural analysis of the fusion mechanism. Nat Commun. 2015; 6:8143.
51. McLellan J S, Chen M, Joyce M G, et al. Structure-based design of a fusion glycoprotein vaccine for respiratory syncytial virus. Science. 2013; 342 (6158): 592-598.
51. Krarup A, Truan D, Furmanova-Hollenstein P, et al. A highly stable prefusion RSV F vaccine derived from structural analysis of the fusion mechanism. Nat Commun. 2015; 6:8143.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES

SEQ ID NO: 1 (amino acid sequence for RSV G truncated CX3C aa 1-86 + 155-206 codon-
optimized (A strain))
MSKNKDQRTAKTLERTWDTLNHLLFISSCLYKLNLKSVAQITLSILAIVISTSLIIAAIIFIASANHKVTPTTAIIQDATSQIKNT
PPSKPNNDFHFEVFNFVPCSICSNNPTCWAICKRIPNKKPGKKTTTKPTKKP
SEQ ID NO: 2 (nucleotide sequence for RSV G truncated CX3C aa 1-86 + 155-206 codon-
optimized (A strain))
ATGAGCAAGAACAAGGACCAGCGGACCGCCAAGACACTGGAAAGAACCTGGGACACCCTGAACCATCTGCTGTTC
ATCAGCAGCTGCCTGTACAAGCTGAACCTGAAGTCTGTGGCCCAGATCACCCTGAGCATCCTGGCCATCGTGATCA
GCACCAGCCTGATCATTGCCGCCATCATCTTTATCGCCAGCGCCAACCACAAAGTGACCCCTACCACAGCCATCATC
CAGGACGCCACAAGCCAGATCAAGAACACCCCTCCAAGCAAGCCCAACAACGACTTCCACTTCGAGGTGTTCAACT
TCGTGCCCTGCAGCATCTGCAGCAACAATCCTACCTGCTGGGCCATCTGCAAGAGAATCCCCAACAAGAAGCCCGG
CAAAAAGACCACCACAAAGCCCACAAAGAAGCCCTAG
SEQ ID NO: 3 (amino acid sequence for truncated RSV G protein amino acid 1-206 (A strain))
MSKNKDQRTTKTLEKTWDTLNHLLFISSCLYKLNLKSIAQITLSILAMIISTSLIIAAIIFIASANHKVTLT
TAIIQDATSQIKNTTPTYLTQNPQLGISFSNLSETTSQTTTILASTTPSVKSTLQSTTVKTKNTTTTKIQ
PSKPTTKQRQNKPPNKPNNDFHFEVFNFVPCSICSNNPTCWAICKRIPNKKPGKKTTTKPTKKP
SEQ ID NO: 4 (nucleotide sequence for truncated RSV G protein amino acid 1-206(A strain))
ATGTCCAAAAACAAGGACCAACGCACCACCAAGACACTAGAAAAG
ACCTGGGACACTCTCAATCATCTATTATTCATATCATCGTGCTTATACAAGTTAAATCTTAAATC
TATAGCACAAATCACATTATCCATTCTGGCAATGATAATCTCAACTTCACTTATAATTGCAGCCA
TCATATTCATAGCCTCGGCAAACCACAAAGTCACACTAACAACTGCAATCATACAAGATGCAAC
AAGCCAGATCAAGAACACAACCCCAACATACCTCACCCAGAATCCCCAGCTTGGAATCAGCTT
CTCCAATCTGTCTGAAACTACATCACAAACCACCACCATACTAGCTTCAACAACACCAAGTGTC
AAGTCAACCCTGCAATCCACAACAGTCAAGACCAAAAACACAACAACAACCAAAATACAACCC
AGCAAGCCCACCACAAAACAACGCCAAAACAAACCACCAAACAAACCCAATAATGATTTTCAC
TTTGAAGTGTTCAACTTTGTACCTTGCAGCATATGCAGCAACAATCCAACCTGCTGGGCTATCT
GTAAAAGAATACCAAACAAAAAACCTGGAAAGAAAACCACCACCAAGCCCACAAAAAAACCA
SEQ ID NO: 5 (amino acid sequence for RSV G protein 1-206 codon-optimized (A strain))
MSKNKDQRTAKTLERTWDTLNHLLFISSCLYKLNLKSVAQITLSILAIVISTSLIIAAIIFIASANHKVTPTTAIIQDATSQIKNT
TPTYLTQNPQLGISPSNPSEITSQITTILASTTPGVKSTLQSTTVKTKNTTTTQTQPSKPTTKQRQNKPPSKPNNDFHFEVF
NFVPCSICSNNPTCWAICKRIPNKKPGKKTTTKPTKKP
SEQ ID NO: 6 (nucleotide sequence for RSV G protein 1-206 codon-optimized (A strain))
ATGAGCAAGAACAAGGACCAGCGGACCGCCAAGACACTGGAAAGAACCTGGGACACCCTGAACCATCTGCTGTT
CATCAGCAGCTGCCTGTACAAGCTGAACCTGAAGTCTGTGGCCCAGATCACCCTGAGCATCCTGGCCATCGTGATC
AGCACCAGCCTGATCATTGCCGCCATCATCTTTATCGCCAGCGCCAACCACAAAGTGACCCCTACCACAGCCATCAT
CCAGGACGCCACAAGCCAGATCAAGAACACCACACCTACCTACCTGACACAGAACCCTCAGCTGGGCATCAGCCCT
AGCAATCCTAGCGAGATCACCTCTCAGATCACCACAATCCTGGCCAGCACAACCCCTGGCGTGAAGTCTACACTGC
AGAGCACCACCGTGAAAACGAAGAATACCACCACCACACAGACCCAGCCTAGCAAGCCTACCACCAAGCAGAGAC
AGAACAAGCCTCCAAGCAAGCCCAACAACGACTTCCACTTCGAGGTGTTCAACTTCGTGCCCTGCAGCATCTGCAG
CAACAATCCTACCTGCTGGGCCATCTGCAAGAGAATCCCCAACAAGAAGCCCGGCAAAAAGACCACCACAAAGCC
CACAAAGAAGCCCTAG
SEQ ID NO: 7 (amino acid sequence for RSV G protein (A strain), NCBI Reference Sequence:
NC_001803.1)
MSKNKDQRTTKTLEKTWDTLNHLLFISSCLYKLNLKSIAQITLSILAMIISTSLIIAAIIFIASANHKVTLT
TAIIQDATSQIKNTTPTYLTQNPQLGISFSNLSETTSQTTTILASTTPSVKSTLQSTTVKTKNTTTTKIQ
PSKPTTKQRQNKPPNKPNNDFHFEVFNFVPCSICSNNPTCWAICKRIPNKKPGKKTTTKPTKKPTIK
TTKKDLKPQTTKPKEVPTTKPTEKPTINTTKTNIRTTLLTNNTTGNPEHTSQKGTLHSTSSDGNPSPS
QVYTTSEYLSQPPSPSNTTNQ
SEQ ID NO: 8 (nucleotide sequence for RSV G protein (A strain), NCBI Reference
Sequence: NC_001803.1)
ATGTCCAAAAACAAGGACCAACGCACCACCAAGACACTAGAAAAGAAATCTATAGCACAAATC
ACATTATCCATTCTGGCAATGATAATCTCAACTTCACTTATAATTGCAGCCATCATATTCATAGCC
TCGGCAAACCACAAAGTCACACTAACAACTGCAATCATACAAGATGCAACAAGCCAGATCAAG
AACACAACCCCAACATACCTCACCCAGAATCCCCAGCTTGGAATCAGCTTCTCCAATCTGTCTGA
AACTACATCACAAACCACCACCATACTAGCTTCAACAACACCAAGTGTCAAGTCAACCCTGCAA
TCCACAACAGTCAAGACCAAAAACACAACAACAACCAAAATACAACCCAGCAAGCCCACCACA
AAACAACGCCAAAACAAACCACCAAACAAACCCAATAATGATTTTCACTTTGAAGTGTTCAACT
TTGTACCTTGCAGCATATGCAGCAACAATCCAACCTGCTGGGCTATCTGTAAAAGAATACCAAA
CAAAAAACCTGGAAAGAAAACCACCACCAAGCCCACAAAAAAACCAACCATCAAGACAACCAA
AAAAGATCTCAAACCTCAAACCACAAAACCAAAGGAAGTACCTACCACCAAGCCCACAGAAAA
GCCAACCATCAACACCACCAAAACAAACATCAGAACTACACTGCTCACCAACAATACCACAGGA
AATCCAGAACACACAAGTCAAAAGGGAACCCTCCACTCAACCTCCTCCGATGGCAATCCAAGCC
CTTCACAAGTCTATACAACATCCGAGTACCTATCACAACCTCCATCTCCATCCAACACAACAAAC
CAGTAG
SEQ ID NO: 9 (amino acid sequence for RSV G protein full length codon-optimized (A strain))
MSKNKDQRTAKTLERTWDTLNHLLFISSCLYKLNLKSVAQITLSILAIVISTSLIIAAIIFIASANHKVTPTTAIIQDATSQIKNT
TPTYLTQNPQLGISPSNPSEITSQITTILASTTPGVKSTLQSTTVKTKNTTTTQTQPSKPTTKQRQNKPPSKPNNDFHFEVF
NFVPCSICSNNPTCWAICKRIPNKKPGKKTTTKPTKKPTLKTTKKDPKPQTTKSKEVPTTKPTEEPTINTTKTNIITTLLTSNT
TGNPELTSQMETFHSTSSEGNPSPSQVSTTSEYPSQPSSPPNTPRQ
SEQ ID NO: 10 (nucleotide sequence for RSV G protein full length codon-
optimized (A strain))
ATGAGCAAGAACAAGGACCAGCGGACCGCCAAGACACTGGAAAGAACCTGGGACACCCTGAACCATCTGCTGTT
CATCAGCAGCTGCCTGTACAAGCTGAACCTGAAGTCTGTGGCCCAGATCACCCTGAGCATCCTGGCCATCGTGATC
AGCACCAGCCTGATCATTGCCGCCATCATCTTTATCGCCAGCGCCAACCACAAAGTGACCCCTACCACAGCCATCAT
CCAGGACGCCACAAGCCAGATCAAGAACACCACACCTACCTACCTGACACAGAACCCTCAGCTGGGCATCAGCCCT
AGCAATCCTAGCGAGATCACCTCTCAGATCACCACAATCCTGGCCAGCACAACCCCTGGCGTGAAGTCTACACTGC
AGAGCACCACCGTGAAAACGAAGAATACCACCACCACACAGACCCAGCCTAGCAAGCCTACCACCAAGCAGAGAC
AGAACAAGCCTCCAAGCAAGCCCAACAACGACTTCCACTTCGAGGTGTTCAACTTCGTGCCCTGCAGCATCTGCAG
CAACAATCCTACCTGCTGGGCCATCTGCAAGAGAATCCCCAACAAGAAGCCCGGCAAAAAGACCACCACAAAGCC
CACAAAGAAGCCCACACTGAAAACCACCAAGAAGGACCCCAAGCCTCAGACCACCAAGTCCAAAGAGGTGCCCAC
CACCAAACCTACCGAGGAACCCACCATCAACACCACTAAGACCAACATCATCACCACACTGCTGACCTCCAACACC
ACCGGCAATCCTGAACTGACCAGCCAGATGGAAACCTTCCACAGCACCTCCAGCGAGGGCAACCCATCTCCTAGTC
AGGTGTCCACCACAAGCGAGTACCCTAGCCAGCCAAGCAGCCCTCCTAACACACCTAGACAGTAG
SEQ ID NO: 11 (amino acid sequence for RSV G A strain CX4C aa 1-86 + 155-206 codon-
optimized)
MSKNKDQRTAKTLERTWDTLNHLLFISSCLYKLNLKSVAQITLSILAIVISTSLIIAAIIFIASANHKVTPTTAIIQDATSQIKNT
PPSKPNNDFHFEVFNFVPCSICSNNPTCWAIACKRIPNKPGKKTTTKPTKKP
SEQ ID NO: 12 (nucleotide sequence for RSV G A strain CX4C aa 1-86 + 155-206 codon-
optimized)
ATGAGCAAGAACAAGGACCAGCGGACCGCCAAGACACTGGAAAGAACCTGGGACACCCTGAACCATCTGCTGTT
CATCAGCAGCTGCCTGTACAAGCTGAACCTGAAGTCTGTGGCCCAGATCACCCTGAGCATCCTGGCCATCGTGATC
AGCACCAGCCTGATCATTGCCGCCATCATCTTTATCGCCAGCGCCAACCACAAAGTGACCCCTACCACAGCCATCAT
CCAGGACGCCACAAGCCAGATCAAGAACACCCCTCCAAGCAAGCCCAACAACGACTTCCACTTCGAGGTGTTCAAC
TTCGTGCCCTGCAGCATCTGCAGCAACAATCCTACCTGCTGGGCCATCGCCTGCAAGAGAATCCCCAACAAGAAGC
CCGGCAAAAAGACCACCACAAAGCCCACAAAGAAGCCCTAG
SEQ ID NO: 13 (amino acid sequence for RSV G protein full length CX4C (A strain))
MSKNKDQRTAKTLERTWDTLNHLLFISSCLYKLNLKSVAQITLSILAIVISTSLIIAAIIFIASANHKVTPTTAIIQDATSQIKNT
TPTYLTQNPQLGISPSNPSEITSQITTILASTTPGVKSTLQSTTVKTKNTTTTQTQPSKPTTKQRQNKPPSKPNNDFHFEVF
NFVPCSICSNNPTCWAIACKRIPNKKPGKKTTTKPTKKPTLKTTKKDPKPQTTKSKEVPTTKPTEEPTINTTKTNIITTLLTS
NTTGNPELTSQMETFHSTSSEGNPSPSQVSTTSEYPSQPSSPPNTPRQ
SEQ ID NO: 14 (nucleotide sequence for RSV G protein full length CX4C (A strain))
ATGAGCAAGAACAAGGACCAGCGGACCGCCAAGACACTGGAAAGAACCTGGGACACCCTGAACCATCTGCTGTT
CATCAGCAGCTGCCTGTACAAGCTGAACCTGAAGTCTGTGGCCCAGATCACCCTGAGCATCCTGGCCATCGTGATC
AGCACCAGCCTGATCATTGCCGCCATCATCTTTATCGCCAGCGCCAACCACAAAGTGACCCCTACCACAGCCATCAT
CCAGGACGCCACAAGCCAGATCAAGAACACCACACCTACCTACCTGACACAGAACCCTCAGCTGGGCATCAGCCCT
AGCAATCCTAGCGAGATCACCTCTCAGATCACCACAATCCTGGCCAGCACAACCCCTGGCGTGAAGTCTACACTGC
AGAGCACCACCGTGAAAACGAAGAATACCACCACCACACAGACCCAGCCTAGCAAGCCTACCACCAAGCAGAGAC
AGAACAAGCCTCCAAGCAAGCCCAACAACGACTTCCACTTCGAGGTGTTCAACTTCGTGCCCTGCAGCATCTGCAG
CAACAATCCTACCTGCTGGGCCATCGCCTGCAAGAGAATCCCCAACAAGAAGCCCGGCAAAAAGACCACCACAAA
GCCCACAAAGAAGCCCACACTGAAAACCACCAAGAAGGACCCCAAGCCTCAGACCACCAAGTCCAAAGAGGTGCC
CACCACCAAACCTACCGAGGAACCCACCATCAACACCACTAAGACCAACATCATCACCACACTGCTGACCTCCAAC
ACCACCGGCAATCCTGAACTGACCAGCCAGATGGAAACCTTCCACAGCACCTCCAGCGAGGGCAACCCATCTCCTA
GTCAGGTGTCCACCACAAGCGAGTACCCTAGCCAGCCAAGCAGCCCTCCTAACACACCTAGACAGTAG
SEQ ID NO: 15 (amino acid sequence for RSV G truncated CX3C aa 1-86 + 155-206 (B strain))
MSKHKNQRTARTLEKTWDTLNHLIVISSCLYRLNLKSIAQIALSVLAMIISTSLIIAAIIFIISANHKVTLTTVTVQTIKNHTEK
NPPKKPKDDYHFEVFNFVPCSICGNNQLCKSICKTIPSNKPKKKPTIKPTNKP
SEQ ID NO: 16 (nucleotide sequence for RSV G truncated CX3C aa 1-86 + 155-206 (B strain))
ATGTCCAAACACAAGAATCAACGCACTGCCAGGACTCTAGAAAAGACCTGGGATACTCTCAATCATCTAATTGTAA
TATCCTCTTGTTTATACAGATTAAATTTAAAATCTATAGCACAAATAGCACTATCAGTTCTGGCAATGATAATCTCAA
CCTCTCTCATAATTGCAGCCATAATATTCATCATCTCTGCCAATCACAAAGTTACACTAACAACGGTCACAGTTCAAA
CAATAAAAAACCACACTGAAAAAAACCCACCAAAAAAACCAAAAGATGATTACCATTTTGAAGTGTTCAACTTCGT
TCCCTGTAGTATATGTGGCAACAATCAACTTTGCAAATCCATCTGTAAAACAATACCAAGCAACAAACCAAAGAAG
AAACCAACCATCAAACCCACAAACAAACCATAG
SEQ ID NO: 17 (amino acid sequence for mutated RSV G truncated CX3C aa 1-
86 + 155-206 (B strain))
MSKHKNQRTARTLEKTWDTLNHLIVISSCLYRLNLKSIAQIALSVLAIVISTSLIIAAIIFIISANHKVTLT
TVTVQTIKNHTEKNPPKKPKDDYHFEVFNFVPCSICGNNQLCKSICKTIPSNKPKKKPTIKPTNKP
SEQ ID NO: 18 (nucleotide sequence for mutated RSV G truncated CX3C aa 1-
86 + 155-206 (B strain))
ATGTCCAAACACAAGAATCAACGCACTGCCAGGACTCTAGAAAAGACCTGGGATACTCTCAAT
CATCTAATTGTAATATCCTCTTGTTTATACAGATTAAATTTAAAATCTATAGCACAAATAGCACT
ATCAGTTCTGGCAATCGTGATCTCAACCTCTCTCATAATTGCAGCCATAATATTCATCATCTCTG
CCAATCACAAAGTTACACTAACAACGGTCACAGTTCAAACAATAAAAAACCACACTGAAAAAA
ACCCACCAAAAAAACCAAAAGATGATTACCATTTTGAAGTGTTCAACTTCGTTCCCTGTAGTAT
ATGTGGCAACAATCAACTTTGCAAATCCATCTGTAAAACAATACCAAGCAACAAACCAAAGAA
GAAACCAACCATCAAACCCACAAACAAACCATAG
SEQ ID NO: 19 (amino acid sequence for RSV G protein B strain, NCBI
Reference Sequence: NC_001781.1)
MSKHKNQRTARTLEKTWDTLNHLIVISSCLYRLNLKSIAQIALSVLAMIISTSLIIAAIIFIISANHKVTLT
TVTVQTIKNHTEKNITTYLTQVPPERVSSSKQPTTTSPIHTNSATTSPNTKSETHHTTAQTKGRTTTS
TQTNKPSTKPRLKNPPKKPKDDYHFEVFNFVPCSICGNNQLCKSICKTIPSNKPKKKPTIKPTNKPTT
KTTNKRDPKTPAKTTKKETTTNPTKKPTLTTTERDTSTSQSTVLDTTTLEHTIQQQSLHSTTPENTPN
STQTPTASEPSTSNSTQNTQSHA
SEQ ID NO: 20 (nucleotide sequence for RSV G protein B strain, NCBI
Reference Sequence: NC_001781.1)
ATGTCCAAACACAAGAATCAACGCACTGCCAGGACTCTAGAAAAGACCTGGGATACTCTCAAT
CATCTAATTGTAATATCCTCTTGTTTATACAGATTAAATTTAAAATCTATAGCACAAATAGCACT
ATCAGTTCTGGCAATGATAATCTCAACCTCTCTCATAATTGCAGCCATAATATTCATCATCTCTG
CCAATCACAAAGTTACACTAACAACGGTCACAGTTCAAACAATAAAAAACCACACTGAAAAAA
ACATCACCACCTACCTTACTCAAGTCCCACCAGAAAGGGTTAGCTCATCCAAACAACCTACAAC
CACATCACCAATCCACACAAATTCAGCCACAACATCACCCAACACAAAGTCAGAAACACACCAC
ACAACAGCACAAACCAAAGGCAGAACCACCACCTCAACACAGACCAACAAGCCGAGCACAAAA
CCACGCCTAAAAAATCCACCAAAAAAACCAAAAGATGATTACCATTTTGAAGTGTTCAACTTCG
TTCCCTGTAGTATATGTGGCAACAATCAACTTTGCAAATCCATCTGTAAAACAATACCAAGCAA
CAAACCAAAGAAGAAACCAACCATCAAACCCACAAACAAACCAACCACCAAAACCACAAACAA
AAGAGACCCAAAAACACCAGCCAAAACGACGAAAAAAGAAACTACCACCAACCCAACAAAAA
AACCAACCCTCACGACCACAGAAAGAGACACCAGCACCTCACAATCCACTGTGCTCGACACAAC
CACATTAGAACACACAATCCAACAGCAATCCCTCCACTCAACCACCCCCGAAAACACACCCAAC
TCCACACAAACACCCACAGCATCCGAGCCCTCTACATCAAATTCCACCCAAAATACCCAATCACA
TGCTTAG
SEQ ID NO: 21 (amino acid sequence for mutated RSV G protein full length (B strain))
MSKHKNQRTARTLEKTWDTLNHLIVISSCLYRLNLKSIAQIALSVLAIVISTSLIIAAIIFIISANHKVTLTT
VTVQTIKNHTEKNITTYLTQVPPERVSSSKQPTTTSPIHTNSATTSPNTKSETHHTTAQTKGRTTTST
QTNKPSTKPRLKNPPKKPKDDYHFEVFNFVPCSICGNNQLCKSICKTIPSNKPKKKPTIKPTNKPTTKT
TNKRDPKTPAKTTKKETTTNPTKKPTLTTTERDTSTSQSTVLDTTTLEHTIQQQSLHSTTPENTPNST
QTPTASEPSTSNSTQNTQSHA
SEQ ID NO: 22 (nucleotide sequence for mutated RSV G protein full length (B strain))
ATGTCCAAACACAAGAATCAACGCACTGCCAGGACTCTAGAAAAGACCTGGGATACTCTCAATCATCTAATTGTAA
TATCCTCTTGTTTATACAGATTAAATTTAAAATCTATAGCACAAATAGCACTATCAGTTCTGGCAATCGTGATCTCAA
CCTCTCTCATAATTGCAGCCATAATATTCATCATCTCTGCCAATCACAAAGTTACACTAACAACGGTCACAGTTCAAA
CAATAAAAAACCACACTGAAAAAAACATCACCACCTACCTTACTCAAGTCCCACCAGAAAGGGTTAGCTCATCCAA
ACAACCTACAACCACATCACCAATCCACACAAATTCAGCCACAACATCACCCAACACAAAGTCAGAAACACACCAC
ACAACAGCACAAACCAAAGGCAGAACCACCACCTCAACACAGACCAACAAGCCGAGCACAAAACCACGCCTAAAA
AATCCACCAAAAAAACCAAAAGATGATTACCATTTTGAAGTGTTCAACTTCGTTCCCTGTAGTATATGTGGCAACAA
TCAACTTTGCAAATCCATCTGTAAAACAATACCAAGCAACAAACCAAAGAAGAAACCAACCATCAAACCCACAAAC
AAACCAACCACCAAAACCACAAACAAAAGAGACCCAAAAACACCAGCCAAAACGACGAAAAAAGAAACTACCACC
AACCCAACAAAAAAACCAACCCTCACGACCACAGAAAGAGACACCAGCACCTCACAATCCACTGTGCTCGACACAA
CCACATTAGAACACACAATCCAACAGCAATCCCTCCACTCAACCACCCCCGAAAACACACCCAACTCCACACAAACA
CCCACAGCATCCGAGCCCTCTACATCAAATTCCACCCAAAATACCCAATCACATGCTTAG
SEQ ID NO: 23 (amino acid sequence for RSV A strain F protein, DS-Cav1 codon-optimized)
MELLILKANAITTILTAVTFCFASGQNITEEFYQSTCSAVSKGYLSALRTGWYTSVITIELSNIKKNKCNGTDAKIKLIKQELD
KYKNAVTELQLLMQSTPATNNRARRELPRFMNYTLNNAKKTNVTLSKKRKRRFLGFLLGVGSAIASGVAVCKVLHLEGE
VNKIKSALLSTNKAVVSLSNGVSVLTFKVLDLKNYIDKQLLPILNKQSCSISNIETVIEFQQKNNRLLEITREFSVNAGVTTPV
STYMLTNSELLSLINDMPITNDQKKLMSNNVQIVRQQSYSIMCIIKEEVLAYVVQLPLYGVIDTPCWKLHTSPLCTTNTKE
GSNICLTRTDRGWYCDNAGSVSFFPQAETCKVQSNRVFCDTMNSLTLPSEVNLCNVDIFNPKYDCKIMTSKTDVSSSVI
TSLGAIVSCYGKTKCTASNKNRGIIKTFSNGCDYVSNKGVDTVSVGNTLYYVNKQEGKSLYVKGEPIINFYDPLVFPSDEF
DASISQVNEKINQSLAFIRKSDELLHNVNAVKSTTNIMITTIIIVIIVILLSLIAVGLLLYCKARSTPVTLSKDQLSGINNIAFSN
SEQ ID NO: 24 (nucleotide sequence for RSV A strain F protein, DS-Cav1 codon-optimized)
cggaaggcccatgaggccagttaattaagaggtaccggatccgccaccATGGAACTGCTGATCCTGAAGGCCAACGCCATCACCAC
CATCCTGACCGCTGTGACCTTCTGCTTCGCCAGCGGCCAGAACATCACCGAGGAATTCTACCAGAGCACCTGTAGC
GCCGTGTCCAAGGGCTACCTGAGCGCCCTGCGGACCGGCTGGTACACCAGCGTGATCACCATCGAGCTGAGCAAC
ATCAAGAAAAACAAGTGCAACGGCACCGACGCCAAGATCAAGCTGATCAAGCAGGAACTGGACAAGTACAAGAA
CGCCGTGACCGAGCTGCAGCTGCTGATGCAGAGCACCCCCGCCACCAACAACCGGGCTAGACGCGAGCTGCCTCG
GTTCATGAACTACACCCTGAACAACGCCAAAAAGACCAACGTGACCCTGAGCAAGAAGCGGAAGCGGCGGTTCCT
GGGCTTCCTGCTGGGCGTGGGCAGCGCCATTGCTAGCGGAGTGGCCGTGTGCAAGGTGCTGCACCTGGAAGGCG
AAGTGAACAAGATCAAGTCCGCCCTGCTGAGCACCAACAAGGCCGTGGTGTCCCTGAGCAACGGCGTGTCCGTGC
TGACCTTCAAGGTGCTGGATCTGAAGAACTACATCGACAAGCAGCTGCTGCCCATCCTGAACAAGCAGAGCTGCA
GCATCAGCAACATCGAGACAGTGATCGAGTTCCAGCAGAAGAACAACCGGCTGCTGGAAATCACCCGCGAGTTCA
GCGTGAACGCCGGCGTGACCACCCCCGTGTCCACCTACATGCTGACCAACAGCGAGCTGCTGAGCCTGATCAACG
ACATGCCCATCACCAACGACCAGAAAAAGCTGATGAGCAACAACGTGCAGATCGTGCGGCAGCAGAGCTACTCCA
TCATGTGCATCATCAAAGAAGAGGTGCTGGCCTACGTGGTGCAGCTGCCCCTGTACGGCGTGATCGACACCCCCT
GCTGGAAGCTGCACACCAGCCCCCTGTGCACCACCAACACCAAAGAGGGCAGCAACATCTGCCTGACCCGGACCG
ACCGGGGCTGGTACTGCGATAATGCCGGCAGCGTGTCATTCTTTCCACAAGCCGAGACATGCAAGGTGCAGAGCA
ACCGGGTGTTCTGCGACACCATGAACAGCCTGACCCTGCCCTCCGAAGTGAACCTGTGCAACGTGGACATCTTCAA
CCCTAAGTACGACTGCAAGATCATGACCTCCAAGACCGACGTGTCCAGCTCCGTGATCACCTCCCTGGGCGCCATC
GTGTCCTGCTACGGCAAGACCAAGTGCACCGCCAGCAACAAGAACCGGGGCATCATCAAGACCTTCAGCAACGGC
TGCGACTACGTGTCCAACAAGGGGGTGGACACCGTGTCCGTGGGCAACACCCTGTACTACGTGAACAAACAGGAA
GGCAAGAGCCTGTACGTGAAGGGCGAGCCCATCATCAACTTCTACGACCCCCTGGTGTTCCCCAGCGACGAGTTC
GACGCCAGCATCAGCCAGGTCAACGAGAAGATCAACCAGAGCCTGGCCTTCATCAGAAAGAGCGACGAGCTGCT
GCACAATGTGAATGCCGTGAAGTCCACCACCAATATCATGATCACCACAATCATCATCGTGATCATCGTCATCCTGC
TGTCCCTGATCGCCGTGGGCCTGCTGCTGTACTGCAAGGCCCGGTCCACCCCTGTGACCCTGTCCAAGGACCAGCT
GAGCGGCATCAACAATATCGCCTTCTCCAACTGActcgagctcatggcgcgcctaggccttgacggccttccg
SEQ ID NO: 25 (coding sequence for RSV A strain F protein, DS-Cav1 codon-optimized)
ATGGAACTGCTGATCCTGAAGGCCAACGCCATCACCACCATCCTGACCGCTGTGACCTTCTGCTTCGCCAGCGGCC
AGAACATCACCGAGGAATTCTACCAGAGCACCTGTAGCGCCGTGTCCAAGGGCTACCTGAGCGCCCTGCGGACCG
GCTGGTACACCAGCGTGATCACCATCGAGCTGAGCAACATCAAGAAAAACAAGTGCAACGGCACCGACGCCAAG
ATCAAGCTGATCAAGCAGGAACTGGACAAGTACAAGAACGCCGTGACCGAGCTGCAGCTGCTGATGCAGAGCAC
CCCCGCCACCAACAACCGGGCTAGACGCGAGCTGCCTCGGTTCATGAACTACACCCTGAACAACGCCAAAAAGAC
CAACGTGACCCTGAGCAAGAAGCGGAAGCGGCGGTTCCTGGGCTTCCTGCTGGGCGTGGGCAGCGCCATTGCTA
GCGGAGTGGCCGTGTGCAAGGTGCTGCACCTGGAAGGCGAAGTGAACAAGATCAAGTCCGCCCTGCTGAGCACC
AACAAGGCCGTGGTGTCCCTGAGCAACGGCGTGTCCGTGCTGACCTTCAAGGTGCTGGATCTGAAGAACTACATC
GACAAGCAGCTGCTGCCCATCCTGAACAAGCAGAGCTGCAGCATCAGCAACATCGAGACAGTGATCGAGTTCCAG
CAGAAGAACAACCGGCTGCTGGAAATCACCCGCGAGTTCAGCGTGAACGCCGGCGTGACCACCCCCGTGTCCACC
TACATGCTGACCAACAGCGAGCTGCTGAGCCTGATCAACGACATGCCCATCACCAACGACCAGAAAAAGCTGATG
AGCAACAACGTGCAGATCGTGCGGCAGCAGAGCTACTCCATCATGTGCATCATCAAAGAAGAGGTGCTGGCCTAC
GTGGTGCAGCTGCCCCTGTACGGCGTGATCGACACCCCCTGCTGGAAGCTGCACACCAGCCCCCTGTGCACCACCA
ACACCAAAGAGGGCAGCAACATCTGCCTGACCCGGACCGACCGGGGCTGGTACTGCGATAATGCCGGCAGCGTG
TCATTCTTTCCACAAGCCGAGACATGCAAGGTGCAGAGCAACCGGGTGTTCTGCGACACCATGAACAGCCTGACC
CTGCCCTCCGAAGTGAACCTGTGCAACGTGGACATCTTCAACCCTAAGTACGACTGCAAGATCATGACCTCCAAGA
CCGACGTGTCCAGCTCCGTGATCACCTCCCTGGGCGCCATCGTGTCCTGCTACGGCAAGACCAAGTGCACCGCCAG
CAACAAGAACCGGGGCATCATCAAGACCTTCAGCAACGGCTGCGACTACGTGTCCAACAAGGGGGTGGACACCG
TGTCCGTGGGCAACACCCTGTACTACGTGAACAAACAGGAAGGCAAGAGCCTGTACGTGAAGGGCGAGCCCATC
ATCAACTTCTACGACCCCCTGGTGTTCCCCAGCGACGAGTTCGACGCCAGCATCAGCCAGGTCAACGAGAAGATCA
ACCAGAGCCTGGCCTTCATCAGAAAGAGCGACGAGCTGCTGCACAATGTGAATGCCGTGAAGTCCACCACCAATA
TCATGATCACCACAATCATCATCGTGATCATCGTCATCCTGCTGTCCCTGATCGCCGTGGGCCTGCTGCTGTACTGC
AAGGCCCGGTCCACCCCTGTGACCCTGTCCAAGGACCAGCTGAGCGGCATCAACAATATCGCCTTCTCCAACTGA
SEQ ID NO: 26 (amino acid sequence for RSV A strain F protein, SC-DM codon-optimized)
MELLILKANAITTILTAVTFCFASGQNITEEFYQSTCSAVSKGYLSALRTGWYTSVITIELSNIKKIKCNGTDAKIKLIKQELDK
YKNAVTELQLLMQSTPATNNQARGSGSGRSLGFLLGVGSAIASGVAVSKVLHLEGEVNKIKSALLSTNKAVVSLSNGVS
VLTSKVLDLKNYIDKQLLPIVNKQSCSIPNIETVIEFQQKNNRLLEITREFSVNAGVTTPVSTYMLTNSELLSLINDMPITND
QKKLMSNNVQIVRQQSYSIMSIIKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSVITSLGAIVSCYGKTKCTASNKNR
GIIKTFSNGCDYVSNKGVDTVSVGNTLYYVNKQEGKSLYVKGEPIINFYDPLVFPSDEFDASISQVNEKINQSLAFIRKSDE
LLHNVNAVKSTTNIMITTIIIVIIVILLSLIAVGLLLYCKARSTPVTLSKDQLSGINNIAFSN
SEQ ID NO: 27 (nucleotide sequence for RSV A strain F protein, SC-DM codon-optimized)
cggaaggcccatgaggccagttaattaagaggtaccggatccgccaccATGGAACTGCTGATCCTGAAGGCCAACGCCATCACCAC
CATCCTGACCGCTGTGACCTTCTGCTTCGCCAGCGGCCAGAACATCACCGAGGAATTCTACCAGAGCACCTGTAGC
GCCGTGTCCAAGGGCTACCTGAGCGCCCTGCGGACCGGCTGGTACACCAGCGTGATCACCATCGAGCTGAGCAAC
ATCAAGAAAATCAAGTGCAACGGCACCGACGCCAAGATCAAGCTGATCAAGCAGGAACTGGACAAGTACAAGAA
CGCCGTGACCGAGCTGCAGCTGCTGATGCAGAGCACCCCCGCCACCAACAACCAGGCTAGA GGC AGC GGA AGC
GGA CGG TCC
CTGGGCTTCCTGCTGGGCGTGGGCAGCGCCATTGCTAGCGGAGTGGCCGTGTCAAAGGTGCTGCACCTGGAAGG
CGAAGTGAACAAGATCAAGTCCGCCCTGCTGAGCACCAACAAGGCCGTGGTGTCCCTGAGCAACGGCGTGTCCGT
GCTGACCAGCAAGGTGCTGGATCTGAAGAACTACATCGACAAGCAGCTGCTGCCCATCGTGAACAAGCAGAGCTG
CAGCATCCCCAACATCGAGACAGTGATCGAGTTCCAGCAGAAGAACAACCGGCTGCTGGAAATCACCCGCGAGTT
CAGCGTGAACGCCGGCGTGACCACCCCCGTGTCCACCTACATGCTGACCAACAGCGAGCTGCTGAGCCTGATCAA
CGACATGCCCATCACCAACGACCAGAAAAAGCTGATGAGCAACAACGTGCAGATCGTGCGGCAGCAGAGCTACTC
CATCATGAGCATCATCAAAGAAGAGGTGCTGGCCTACGTGGTGCAGCTGCCCCTGTACGGCGTGATCGACACCCC
CTGCTGGAAGCTGCACACCAGCCCCCTGTGCACCACCAACACCAAAGAGGGCAGCAACATCTGCCTGACCCGGAC
CGACCGGGGCTGGTACTGCGATAATGCCGGCAGCGTGTCATTCTTTCCACAAGCCGAGACATGCAAGGTGCAGAG
CAACCGGGTGTTCTGCGACACCATGAACAGCCTGACCCTGCCCTCCGAAGTGAACCTGTGCAACGTGGACATCTTC
AACCCTAAGTACGACTGCAAGATCATGACCTCCAAGACCGACGTGTCCAGCTCCGTGATCACCTCCCTGGGCGCCA
TCGTGTCCTGCTACGGCAAGACCAAGTGCACCGCCAGCAACAAGAACCGGGGCATCATCAAGACCTTCAGCAACG
GCTGCGACTACGTGTCCAACAAGGGGGTGGACACCGTGTCCGTGGGCAACACCCTGTACTACGTGAACAAACAG
GAAGGCAAGAGCCTGTACGTGAAGGGCGAGCCCATCATCAACTTCTACGACCCCCTGGTGTTCCCCAGCGACGAG
TTCGACGCCAGCATCAGCCAGGTCAACGAGAAGATCAACCAGAGCCTGGCCTTCATCAGAAAGAGCGACGAGCTG
CTGCACAATGTGAATGCCGTGAAGTCCACCACCAATATCATGATCACCACAATCATCATCGTGATCATCGTCATCCT
GCTGTCCCTGATCGCCGTGGGCCTGCTGCTGTACTGCAAGGCCCGGTCCACCCCTGTGACCCTGTCCAAGGACCAG
CTGAGCGGCATCAACAATATCGCCTTCTCCAACTGActcgagctcatggcgcgcctaggccttgacggccttccg
SEQ ID NO: 28 (coding sequence for RSV A strain F protein, SC-DM codon-optimized)
ATGGAACTGCTGATCCTGAAGGCCAACGCCATCACCACCATCCTGACCGCTGTGACCTTCTGCTTCGCCAGCGGCC
AGAACATCACCGAGGAATTCTACCAGAGCACCTGTAGCGCCGTGTCCAAGGGCTACCTGAGCGCCCTGCGGACCG
GCTGGTACACCAGCGTGATCACCATCGAGCTGAGCAACATCAAGAAAATCAAGTGCAACGGCACCGACGCCAAGA
TCAAGCTGATCAAGCAGGAACTGGACAAGTACAAGAACGCCGTGACCGAGCTGCAGCTGCTGATGCAGAGCACC
CCCGCCACCAACAACCAGGCTAGA GGC AGC GGA AGC GGA CGG TCC
CTGGGCTTCCTGCTGGGCGTGGGCAGCGCCATTGCTAGCGGAGTGGCCGTGTCAAAGGTGCTGCACCTGGAAGG
CGAAGTGAACAAGATCAAGTCCGCCCTGCTGAGCACCAACAAGGCCGTGGTGTCCCTGAGCAACGGCGTGTCCGT
GCTGACCAGCAAGGTGCTGGATCTGAAGAACTACATCGACAAGCAGCTGCTGCCCATCGTGAACAAGCAGAGCTG
CAGCATCCCCAACATCGAGACAGTGATCGAGTTCCAGCAGAAGAACAACCGGCTGCTGGAAATCACCCGCGAGTT
CAGCGTGAACGCCGGCGTGACCACCCCCGTGTCCACCTACATGCTGACCAACAGCGAGCTGCTGAGCCTGATCAA
CGACATGCCCATCACCAACGACCAGAAAAAGCTGATGAGCAACAACGTGCAGATCGTGCGGCAGCAGAGCTACTC
CATCATGAGCATCATCAAAGAAGAGGTGCTGGCCTACGTGGTGCAGCTGCCCCTGTACGGCGTGATCGACACCCC
CTGCTGGAAGCTGCACACCAGCCCCCTGTGCACCACCAACACCAAAGAGGGCAGCAACATCTGCCTGACCCGGAC
CGACCGGGGCTGGTACTGCGATAATGCCGGCAGCGTGTCATTCTTTCCACAAGCCGAGACATGCAAGGTGCAGAG
CAACCGGGTGTTCTGCGACACCATGAACAGCCTGACCCTGCCCTCCGAAGTGAACCTGTGCAACGTGGACATCTTC
AACCCTAAGTACGACTGCAAGATCATGACCTCCAAGACCGACGTGTCCAGCTCCGTGATCACCTCCCTGGGCGCCA
TCGTGTCCTGCTACGGCAAGACCAAGTGCACCGCCAGCAACAAGAACCGGGGCATCATCAAGACCTTCAGCAACG
GCTGCGACTACGTGTCCAACAAGGGGGTGGACACCGTGTCCGTGGGCAACACCCTGTACTACGTGAACAAACAG
GAAGGCAAGAGCCTGTACGTGAAGGGCGAGCCCATCATCAACTTCTACGACCCCCTGGTGTTCCCCAGCGACGAG
TTCGACGCCAGCATCAGCCAGGTCAACGAGAAGATCAACCAGAGCCTGGCCTTCATCAGAAAGAGCGACGAGCTG
CTGCACAATGTGAATGCCGTGAAGTCCACCACCAATATCATGATCACCACAATCATCATCGTGATCATCGTCATCCT
GCTGTCCCTGATCGCCGTGGGCCTGCTGCTGTACTGCAAGGCCCGGTCCACCCCTGTGACCCTGTCCAAGGACCAG
CTGAGCGGCATCAACAATATCGCCTTCTCCAACTGA
SEQ ID NO: 29 (amino acid sequence for RSV A strain F protein, SC-TM codon-optimized)
MELLILKANAITTILTAVTFCFASGQNITEEFYQSTCSAVSKGYLSALRTGWYTSVITIELSNIKKIKCNGTDAKIKLIKQELDK
YKNAVTELQLLMQSTPATNNQARGSGSGRSLGFLLGVGSAIASGVAVSKVLHLEGEVNKIKSALLSTNKAVVSLSNGVS
VLTSKVLDLKNYIDKQLLPIVNKQSCSIPNIETVIEFQQKNNRLLEITREFSVNAGVTTPVSTYMLTNSELLSLINDMPITND
QKKLMSNNVQIVRQQSYSIMSIIKEEVLAYVVQLPLYGVIDTPCWKLHTSPLCTTNTKEGSNICLTRTDRGWYCDNAGS
VSFFPQAETCKVQSNRVFCDTMNSLTLPSEVNLCNVDIFNPKYDCKIMTSKTDVSSSVITSLGAIVSCYGKTKCTASNKNR
GIIKTFSNGCDYVSNKGVDTVSVGNTLYYVNKQEGKSLYVKGEPIINFYDPLVFPSDQFDASISQVNEKINQSLAFIRKSDE
LLHNVNAVKSTTNIMITTIIIVIIVILLSLIAVGLLLYCKARSTPVTLSKDQLSGINNIAFSN
SEQ ID NO: 30 (nucleotide sequence for RSV A strain F protein, SC-TM codon-optimized)
cggaaggcccatgaggccagttaattaagaggtaccggatccgccaccATGGAACTGCTGATCCTGAAGGCCA
ACGCCATCACCACCATCCTGACCGCTGTGACCTTCTGCTTCGCCAGCGGCCAGAACATCACCGA
GGAATTCTACCAGAGCACCTGTAGCGCCGTGTCCAAGGGCTACCTGAGCGCCCTGCGGACCG
GCTGGTACACCAGCGTGATCACCATCGAGCTGAGCAACATCAAGAAAATCAAGTGCAACGGC
ACCGACGCCAAGATCAAGCTGATCAAGCAGGAACTGGACAAGTACAAGAACGCCGTGACCGA
GCTGCAGCTGCTGATGCAGAGCACCCCCGCCACCAACAACCAGGCTAGA GGC AGC GGA
AGC GGA CGG TCC
CTGGGCTTCCTGCTGGGCGTGGGCAGCGCCATTGCTAGCGGAGTGGCCGTGTCAAAGGTGCT
GCACCTGGAAGGCGAAGTGAACAAGATCAAGTCCGCCCTGCTGAGCACCAACAAGGCCGTGG
TGTCCCTGAGCAACGGCGTGTCCGTGCTGACCAGCAAGGTGCTGGATCTGAAGAACTACATC
GACAAGCAGCTGCTGCCCATCGTGAACAAGCAGAGCTGCAGCATCCCCAACATCGAGACAGT
GATCGAGTTCCAGCAGAAGAACAACCGGCTGCTGGAAATCACCCGCGAGTTCAGCGTGAACG
CCGGCGTGACCACCCCCGTGTCCACCTACATGCTGACCAACAGCGAGCTGCTGAGCCTGATCA
ACGACATGCCCATCACCAACGACCAGAAAAAGCTGATGAGCAACAACGTGCAGATCGTGCGG
CAGCAGAGCTACTCCATCATGAGCATCATCAAAGAAGAGGTGCTGGCCTACGTGGTGCAGCT
GCCCCTGTACGGCGTGATCGACACCCCCTGCTGGAAGCTGCACACCAGCCCCCTGTGCACCAC
CAACACCAAAGAGGGCAGCAACATCTGCCTGACCCGGACCGACCGGGGCTGGTACTGCGATA
ATGCCGGCAGCGTGTCATTCTTTCCACAAGCCGAGACATGCAAGGTGCAGAGCAACCGGGTG
TTCTGCGACACCATGAACAGCCTGACCCTGCCCTCCGAAGTGAACCTGTGCAACGTGGACATC
TTCAACCCTAAGTACGACTGCAAGATCATGACCTCCAAGACCGACGTGTCCAGCTCCGTGATC
ACCTCCCTGGGCGCCATCGTGTCCTGCTACGGCAAGACCAAGTGCACCGCCAGCAACAAGAAC
CGGGGCATCATCAAGACCTTCAGCAACGGCTGCGACTACGTGTCCAACAAGGGGGTGGACAC
CGTGTCCGTGGGCAACACCCTGTACTACGTGAACAAACAGGAAGGCAAGAGCCTGTACGTGA
AGGGCGAGCCCATCATCAACTTCTACGACCCCCTGGTGTTCCCCAGCGACCAGTTCGACGCCA
GCATCAGCCAGGTCAACGAGAAGATCAACCAGAGCCTGGCCTTCATCAGAAAGAGCGACGAG
CTGCTGCACAATGTGAATGCCGTGAAGTCCACCACCAATATCATGATCACCACAATCATCATCG
TGATCATCGTCATCCTGCTGTCCCTGATCGCCGTGGGCCTGCTGCTGTACTGCAAGGCCCGGTC
CACCCCTGTGACCCTGTCCAAGGACCAGCTGAGCGGCATCAACAATATCGCCTTCTCCAACTG
Actcgagctcatggcgcgcctaggccttgacggccttccg
SEQ ID NO: 31 (coding sequence for RSV A strain F protein, SC-TM codon-optimized)
ATGGAACTGCTGATCCTGAAGGCCAACGCCATCACCACCATCCTGACCGCTGTGACCTTCTGCT
TCGCCAGCGGCCAGAACATCACCGAGGAATTCTACCAGAGCACCTGTAGCGCCGTGTCCAAG
GGCTACCTGAGCGCCCTGCGGACCGGCTGGTACACCAGCGTGATCACCATCGAGCTGAGCAA
CATCAAGAAAATCAAGTGCAACGGCACCGACGCCAAGATCAAGCTGATCAAGCAGGAACTGG
ACAAGTACAAGAACGCCGTGACCGAGCTGCAGCTGCTGATGCAGAGCACCCCCGCCACCAAC
AACCAGGCTAGA GGC AGC GGA AGC GGA CGG TCC
CTGGGCTTCCTGCTGGGCGTGGGCAGCGCCATTGCTAGCGGAGTGGCCGTGTCAAAGGTGCT
GCACCTGGAAGGCGAAGTGAACAAGATCAAGTCCGCCCTGCTGAGCACCAACAAGGCCGTGG
TGTCCCTGAGCAACGGCGTGTCCGTGCTGACCAGCAAGGTGCTGGATCTGAAGAACTACATC
GACAAGCAGCTGCTGCCCATCGTGAACAAGCAGAGCTGCAGCATCCCCAACATCGAGACAGT
GATCGAGTTCCAGCAGAAGAACAACCGGCTGCTGGAAATCACCCGCGAGTTCAGCGTGAACG
CCGGCGTGACCACCCCCGTGTCCACCTACATGCTGACCAACAGCGAGCTGCTGAGCCTGATCA
ACGACATGCCCATCACCAACGACCAGAAAAAGCTGATGAGCAACAACGTGCAGATCGTGCGG
CAGCAGAGCTACTCCATCATGAGCATCATCAAAGAAGAGGTGCTGGCCTACGTGGTGCAGCT
GCCCCTGTACGGCGTGATCGACACCCCCTGCTGGAAGCTGCACACCAGCCCCCTGTGCACCAC
CAACACCAAAGAGGGCAGCAACATCTGCCTGACCCGGACCGACCGGGGCTGGTACTGCGATA
ATGCCGGCAGCGTGTCATTCTTTCCACAAGCCGAGACATGCAAGGTGCAGAGCAACCGGGTG
TTCTGCGACACCATGAACAGCCTGACCCTGCCCTCCGAAGTGAACCTGTGCAACGTGGACATC
TTCAACCCTAAGTACGACTGCAAGATCATGACCTCCAAGACCGACGTGTCCAGCTCCGTGATC
ACCTCCCTGGGCGCCATCGTGTCCTGCTACGGCAAGACCAAGTGCACCGCCAGCAACAAGAAC
CGGGGCATCATCAAGACCTTCAGCAACGGCTGCGACTACGTGTCCAACAAGGGGGTGGACAC
CGTGTCCGTGGGCAACACCCTGTACTACGTGAACAAACAGGAAGGCAAGAGCCTGTACGTGA
AGGGCGAGCCCATCATCAACTTCTACGACCCCCTGGTGTTCCCCAGCGACCAGTTCGACGCCA
GCATCAGCCAGGTCAACGAGAAGATCAACCAGAGCCTGGCCTTCATCAGAAAGAGCGACGAG
CTGCTGCACAATGTGAATGCCGTGAAGTCCACCACCAATATCATGATCACCACAATCATCATCG
TGATCATCGTCATCCTGCTGTCCCTGATCGCCGTGGGCCTGCTGCTGTACTGCAAGGCCCGGTC
CACCCCTGTGACCCTGTCCAAGGACCAGCTGAGCGGCATCAACAATATCGCCTTCTCCAACTG
A
SEQ ID NO: 32 (amino acid sequence for RSV F truncated, amino acid 496-574 codon-
optimized (A strain))
MELLILKANAITTILTAVTFCFASGQNITEEFYQSNEKINQSLAFIRKSDELLHNVNAVKSTTNIMITTIIIVIIVILLSLIAVGLLL
YCKARSTPVTLSKDQLSGINNIAFSN
SEQ ID NO: 33 (coding nucleotide sequence for RSV F truncated, amino acid 496-574 codon-
optimized (A strain))
ATGGAACTGCTGATCCTGAAGGCCAACGCCATCACCACCATCCTGACCGCTGTGACCTTCTGCT
TCGCCAGCGGCCAGAACATCACCGAGGAATTCTACCAGAGCAACGAGAAGATCAACCAGAGC
CTGGCCTTCATCAGAAAGAGCGACGAGCTGCTGCACAATGTGAATGCCGTGAAGTCCACCACC
AATATCATGATCACCACAATCATCATCGTGATCATCGTCATCCTGCTGTCCCTGATCGCCGTGG
GCCTGCTGCTGTACTGCAAGGCCCGGTCCACCCCTGTGACCCTGTCCAAGGACCAGCTGAGCG
GCATCAACAATATCGCCTTCTCCAACTGA
SEQ ID NO: 34 (amino acid sequence for RSV F protein, NCBI Reference Sequence:
NC_001803.1)
MELPILKTNAITAILAAVTLCFASSQNITEEFYQTTCSAVSKGYLSALRTGWYTSVITIELSNIKENKCN
GTDAKVKLIKQELDKYKSAVTELQLLMQSTPATNNRARRELPRFMNYTLNNTKNTNVTLSKKRKRR
FLGFLLGVGSAIASGIAVSKVLHLEGEVNKIKSALLSTNKAVVSLSNGVSVLTSKVLDLKNYIDKQLLPI
VNKQSCSISNIETVIEFQQKNNRLLEITREFSVNAGVTTPVSTYMLTNSELLSLINDMPITNDQKKLM
SNNVQIVRQQSYSIMSIIKEEVLAYVVQLPLYGVIDTPCWKLHTSPLCTTNTKEGSNICLTRTDRGW
YCDNAGSVSFFPLAETCKVQSNRVFCDTMNSLTLPSEVNLCNIDIFNPKYDCKIMTSKTDVSSSVITS
LGAIVSCYGKTKCTASNKNRGIIKTFSNGCDYVSNKGVDTVSVGNTLYYVNKQEGKSLYVKGEPIIN
FYDPLVFPSDEFDASISQVNEKINQSLAFIRKSDELLHNVNAGKSTINIMITTIIIVIIVILLSLIAVGLLLY
CKARSTPVTLSKDQLSGINNIAFSN
SEQ ID NO: 35 (nucleotide sequence for RSV F protein, NCBI Reference Sequence:
NC_001803.1)
ATGGAGTTGCCAATCCTCAAAACAAATGCAATTACCGCAATCCTTGCTGCAGTCACACTCTGTTT
TGCTTCCAGTCAAAACATCACTGAAGAATTTTATCAAACAACA
TGCAGTGCAGTCAGCAAAGGCTATCTTAGTGCTCTAAGAACTGGTTGGTATACTAGTGTTATAA
CTATAGAATTAAGTAATATCAAGGAAAATAAGTGTAATGGAACAGACGCTAAGGTAAAATTGA
TAAAACAAGAATTAGATAAATATAAAAGTGCTGTAACAGAATTGCAGTTGCTCATGCAAAGCA
CACCGGCAACCAACAATCGAGCCAGAAGAGAACTACCAAGGTTTATGAATTATACACTCAACA
ATACCAAAAATACCAATGTAACATTAAGCAAGAAAAGGAAAAGAAGATTTCTTGGCTTTTTGTT
AGGTGTTGGATCTGCAATCGCCAGTGGCATTGCTGTATCTAAGGTCCTGCACCTAGAAGGGGA
AGTGAACAAAATCAAAAGTGCTCTACTATCCACAAACAAGGCTGTAGTCAGCTTATCAAATGGA
GTTAGTGTCTTAACCAGCAAAGTGTTAGACCTCAAAAACTATATAGATAAACAGTTGTTACCTA
TTGTGAACAAGCAAAGCTGTAGCATATCAAAC
ATTGAAACTGTGATAGAGTTCCAACAAAAGAACAACAGACTACTAGAGATTACCAGGGAATTT
AGTGTTAATGCAGGTGTAACTACACCTGTAAGCACTTATATGTTAACAAATAGTGAATTATTAT
CATTAATCAATGATATGCCTATAACAAATGATCAGAAAAAGTTAATGTCCAACAATGTTCAAAT
AGTTAGACAGCAAAGTTACTCTATCATGTCCATAATAAAGGAGGAAGTCTTAGCATATGTAGTA
CAATTACCACTATATGGTGTAATAGATACACCTTGTTGGAAACTGCACACATCCCCTCTATGTAC
AACCAACACAAAGGAAGGGTCCAACATCTGTTTAACAAGAACCGACAGAGGATGGTACTGTGA
CAATGCAGGATCAGTATCTTTCTTCCCACTAGCTGAAACATGTAAAGTTCAATCGAATCGAGTA
TTTTGTGACACAATGAACAGTTTAACATTACCAAGTGAAGTAAATCTCTGCAACATTGACATATT
CAACCCCAAATATGATTGCAAAATTATG
ACTTCAAAAACAGATGTAAGCAGCTCCGTTATCACATCTCTAGGAGCCATTGTGTCATGCTATG
GCAAAACTAAATGTACAGCATCCAATAAAAATCGTGGAATCATAAAGACATTTTCTAACGGGTG
CGATTATGTATCAAATAAGGGGGTTGACACTGTGTCTGTAGGTAATACATTATATTATGTAAAT
AAGCAAGAAGGCAAAAGTCTCTATGTAAAAGGTGAACCAATAATAAATTTCTATGACCCATTA
GTGTTCCCCTCTGATGAATTTGATGCATCAATATCTCAAGTCAATGAGAAGATTAACCAGAGCC
TAGCATTTATTCGTAAATCCGATGAATTATTACATAATGTAAATGCTGGTAAATCCACCATAAAT
ATCATGATAACTACTATAATTATAGTGATTATAGTAATATTGTTATCATTAATTGCCGTTGGACT
GCTCCTATACTGCAAGGCCAGAAGCACACCAGTCACACTAAGCAAGGATCAACTGAGTGGTAT
AAATAATATTGCATTTAGTAACTAAAACGGGTGCGATTATGTATCAAATAAGGGGGTTGACACT
GTGTCTGTAGGTAATACATTATATTATGTAAATAAGCAAGAAGGCAAAAGTCTCTATGTAAAAG
GTGAACCAATAATAAATTTCTATGACCCATTAGTGTTCCCCTCTGATGAATTTGATGCATCAATA
TCTCAAGTCAATGAGAAGATTAACCAGAGCCTAGCATTTATTCGTAAATCCGATGAATTATTAC
ATAATGTAAATGCTGGTAAATCCACCATAAATATCATGATAACTACTATAATTATAGTGATTAT
AGTAATATTGTTATCATTAATTGCCGTTGGACTGCTCCTATACTGCAAGGCCAGAAGCACACCA
GTCACACTAAGCAAGGATCAACTGAGTGGTATAAATAATATTGCATTTAGTAACTAA
SEQ ID NO: 36 (amino acid sequence for full length RSV F protein (A strain), NCBI Reference
Sequence: NC_001803.1)
MELPILKTNA ITAILAAVTL CFASSQNITE EFYQTTCSAV SKGYLSALRT GWYTSVITIE
LSNIKENKCN GTDAKVKLIK QELDKYKSAV TELQLLMQST PATNNRARRE LPRFMNYTLN
NTKNTNVTLS KKRKRRFLGF LLGVGSAIAS GIAVSKVLHL EGEVNKIKSA LLSTNKAVVS
LSNGVSVLTS KVLDLKNYID KQLLPIVNKQ SCSISNIETV IEFQQKNNRL LEITREFSVN
AGVTTPVSTY MLTNSELLSL INDMPITNDQ KKLMSNNVQI VRQQSYSIMS IIKEEVLAYV
VQLPLYGVID TPCWKLHTSP LCTTNTKEGS NICLTRTDRG WYCDNAGSVS FFPLAETCKV
QSNRVFCDTM NSLTLPSEVN LCNIDIFNPK YDCKIMTSKT DVSSSVITSL GAIVSCYGKT
KCTASNKNRG IIKTFSNGCD YVSNKGVDTV SVGNTLYYVN KQEGKSLYVK GEPIINFYDP
LVFPSDEFDA SISQVNEKIN QSLAFIRKSD ELLHNVNAGK STINIMITTI IIVIIVILLS
LIAVGLLLYC KARSTPVTLS KDQLSGINNI AFSN
SEQ ID NO: 37 (amino acid sequence for full length RSV F protein, codon-optimized
(A strain))
MELLILKANAITTILTAVTFCFASGQNITEEFYQSTCSAVSKGYLSALRTGWYTSVITIELSNIKKNKCNGTDAKIKLIKQELD
KYKNAVTELQLLMQSTPATNNRARRELPRFMNYTLNNAKKTNVTLSKKRKRRFLGFLLGVGSAIASGVAVSKVLHLEGE
VNKIKSALLSTNKAVVSLSNGVSVLTSKVLDLKNYIDKQLLPIVNKQSCSISNIETVIEFQQKNNRLLEITREFSVNAGVTTP
VSTYMLTNSELLSLINDMPITNDQKKLMSNNVQIVRQQSYSIMSIIKEEVLAYVVQLPLYGVIDTPCWKLHTSPLCTTNTK
EGSNICLTRTDRGWYCDNAGSVSFFPQAETCKVQSNRVFCDTMNSLTLPSEVNLCNVDIFNPKYDCKIMTSKTDVSSSV
ITSLGAIVSCYGKTKCTASNKNRGIIKTFSNGCDYVSNKGVDTVSVGNTLYYVNKQEGKSLYVKGEPIINFYDPLVFPSDEF
DASISQVNEKINQSLAFIRKSDELLHNVNAVKSTTNIMITTIIIVIIVILLSLIAVGLLLYCKARSTPVTLSKDQLSGINNIAFSN
SEQ ID NO: 38 (nucleotide sequence for full length RSV F protein, codon-
optimized (A strain))
ATGGAACTGCTGATCCTGAAGGCCAACGCCATCACCACCATCCTGACCGCTGTGACCTTCTGCTTCGCCAGCGGCC
AGAACATCACCGAGGAATTCTACCAGAGCACCTGTAGCGCCGTGTCCAAGGGCTACCTGAGCGCCCTGCGGACCG
GCTGGTACACCAGCGTGATCACCATCGAGCTGAGCAACATCAAGAAAAACAAGTGCAACGGCACCGACGCCAAG
ATCAAGCTGATCAAGCAGGAACTGGACAAGTACAAGAACGCCGTGACCGAGCTGCAGCTGCTGATGCAGAGCAC
CCCCGCCACCAACAACCGGGCTAGACGCGAGCTGCCTCGGTTCATGAACTACACCCTGAACAACGCCAAAAAGAC
CAACGTGACCCTGAGCAAGAAGCGGAAGCGGCGGTTCCTGGGCTTCCTGCTGGGCGTGGGCAGCGCCATTGCTA
GCGGAGTGGCCGTGTCAAAGGTGCTGCACCTGGAAGGCGAAGTGAACAAGATCAAGTCCGCCCTGCTGAGCACC
AACAAGGCCGTGGTGTCCCTGAGCAACGGCGTGTCCGTGCTGACCAGCAAGGTGCTGGATCTGAAGAACTACATC
GACAAGCAGCTGCTGCCCATCGTGAACAAGCAGAGCTGCAGCATCAGCAACATCGAGACAGTGATCGAGTTCCAG
CAGAAGAACAACCGGCTGCTGGAAATCACCCGCGAGTTCAGCGTGAACGCCGGCGTGACCACCCCCGTGTCCACC
TACATGCTGACCAACAGCGAGCTGCTGAGCCTGATCAACGACATGCCCATCACCAACGACCAGAAAAAGCTGATG
AGCAACAACGTGCAGATCGTGCGGCAGCAGAGCTACTCCATCATGAGCATCATCAAAGAAGAGGTGCTGGCCTAC
GTGGTGCAGCTGCCCCTGTACGGCGTGATCGACACCCCCTGCTGGAAGCTGCACACCAGCCCCCTGTGCACCACCA
ACACCAAAGAGGGCAGCAACATCTGCCTGACCCGGACCGACCGGGGCTGGTACTGCGATAATGCCGGCAGCGTG
TCATTCTTTCCACAAGCCGAGACATGCAAGGTGCAGAGCAACCGGGTGTTCTGCGACACCATGAACAGCCTGACC
CTGCCCTCCGAAGTGAACCTGTGCAACGTGGACATCTTCAACCCTAAGTACGACTGCAAGATCATGACCTCCAAGA
CCGACGTGTCCAGCTCCGTGATCACCTCCCTGGGCGCCATCGTGTCCTGCTACGGCAAGACCAAGTGCACCGCCAG
CAACAAGAACCGGGGCATCATCAAGACCTTCAGCAACGGCTGCGACTACGTGTCCAACAAGGGGGTGGACACCG
TGTCCGTGGGCAACACCCTGTACTACGTGAACAAACAGGAAGGCAAGAGCCTGTACGTGAAGGGCGAGCCCATC
ATCAACTTCTACGACCCCCTGGTGTTCCCCAGCGACGAGTTCGACGCCAGCATCAGCCAGGTCAACGAGAAGATCA
ACCAGAGCCTGGCCTTCATCAGAAAGAGCGACGAGCTGCTGCACAATGTGAATGCCGTGAAGTCCACCACCAATA
TCATGATCACCACAATCATCATCGTGATCATCGTCATCCTGCTGTCCCTGATCGCCGTGGGCCTGCTGCTGTACTGC
AAGGCCCGGTCCACCCCTGTGACCCTGTCCAAGGACCAGCTGAGCGGCATCAACAATATCGCCTTCTCCAACTGA
SEQ ID NO: 39 (amino acid sequence for RSV M protein, NCBI Reference
Sequence: NC_001803.1)
METYVNKLHEGSTYTAAVQYNVLEKDDDPASLTIWVPMFQSSMPADLLIKELANVNILVKQISTPN
GPSLRVMINSRSAVLAQMPSKFTICANVSLDERSKLAYDVTTPCEIKACSLTCLKSKNMLTTVKDLT
MKTLNPTHDIIALCEFENIVTSKKVIIPTYLRSISVRNKDLNTLENITTTEFKNAITNAKIIPYSGLLLVITV
TDNKGAFKYIKPQSQFIVDLGAYLEKESIYYVTTNWKHTATRFAIKPMED
SEQ ID NO: 40 (nucleotide sequence for RSV M protein, NCBI Reference
Sequence: NC_001803.1)
ATGGAAACATACGTGAACAAACTTCACGAAGGCTCCACATACACAGCTGCTGTTCAATACAATG
TCTTAGAAAAAGACGATGACCCTGCATCACTTACAATATGGGTGCCCATGTTCCAATCATCCAT
GCCAGCAGATTTACTTATAAAAGAACTAGCTAATGTCAACATACTAGTGAAACAAATATCCACA
CCCAATGGACCTTCATTAAGAGTCATGATAAACTCAAGAAGTGCAGTGCTAGCACAAATGCCCA
GCAAATTTACCATATGTGCCAATGTGTCCTTGGATGAAAGAAGCAAGCTGGCATATGATGTAA
CCACACCCTGTGAAATCAAGGCATGTAGTCTAACATGCCTAAAATCAAAAAATATGTTAACTAC
AGTTAAAGATCTCACTATGAAAACACTCAACCCAACACATGACATCATTGCTTTATGTGAATTT
GAAAATATAGTAACATCAAAAAAAGTCATAATACCAACATACCTAAGATCCATCAGTGTCAGAA
ATAAAGATCTGAACACACTTGAAAATATA
ACAACCACTGAATTCAAAAATGCCATCACAAATGCAAAAATCATCCCTTACTCAGGATTACTGTT
AGTCATCACAGTGACTGACAACAAAGGAGCATTCAAATACATAAAGCCACAAAGTCAATTTAT
AGTAGATCTTGGAGCTTACCTAGAAAAAGAAAGTATATATTATGTTACAACAAATTGGAAGCA
CACAGCTACACGATTTGCAATCAAACCCATGGAAGATTAA
SEQ ID NO: 41 (amino acid sequence for RSV M protein codon-optimized (A
strain))
MFQSSMPADLLIKDSTYTAAVQYNVLEKDDDPASLTIWVPMFQSSMPADLLIKELANVNILVKQIS
TPKGPSLRVMINSRSAVLAQMPSKFTICANVSLDERSKLAYDVTTPCEIKACSLTCLKSKNMLTTVK
DLTMKTLNPTHDIIALCEFENIVTSKKVIIPTYLRSISVRNKDLNTLENITTTEFKNAITNAKIIPYSGLLL
VITVTDNKGAFKYIKPQSQFIVDLGAYLEKESIYYVTTNWKHTATRFAIKPMED
SEQ ID NO: 42 (nucleotide sequence for RSV M protein codon-optimized (A
strain))
ATGTTCCAGAGCAGCATGCCCGCCGACCTGCTGATCAAAGACAGCACCTACACAGCCGCCGTG
CAGTACAACGTGCTGGAAAAGGACGACGACCCCGCCAGCCTGACCATCTGGGTGCCCATGTT
CCAGAGCAGCATGCCCGCCGACCTGCTGATCAAAGAACTGGCCAACGTGAACATCCTGGTCA
AGCAGATCAGCACCCCCAAGGGCCCCAGCCTGAGAGTGATGATCAACAGCCGCAGCGCCGTG
CTGGCCCAGATGCCCAGCAAGTTCACCATCTGCGCCAACGTGTCCCTGGACGAGCGGAGCAA
GCTGGCCTACGACGTGACCACCCCCTGCGAGATCAAGGCCTGCAGCCTGACCTGCCTGAAGTC
CAAGAACATGCTGACCACCGTGAAGGACCTGACCATGAAGACCCTGAACCCCACCCACGACAT
CATTGCCCTGTGCGAGTTCGAGAACATCGTGACCAGCAAGAAAGTGATCATCCCCACCTACCT
GCGGAGCATCAGCGTGCGGAACAAGGACCTGAACACCCTGGAAAACATCACCACCACCGAGT
TCAAGAACGCCATTACCAACGCCAAGATCATCCCCTACAGCGGCCTGCTGCTGGTCATCACCG
TGACCGACAACAAGGGCGCCTTCAAGTACATCAAGCCCCAGAGCCAGTTCATCGTGGACCTG
GGCGCCTACCTGGAAAAAGAATCCATCTACTACGTCACCACCAACTGGAAGCACACCGCCACC
AGATTCGCCATCAAGCCCATGGAAGATTGA
SEQ ID NO: 43 (amino acid sequence for RSV M2-1 protein, NCBI Reference
Sequence: NC_001803.1)
MSRRNPCKFEIRGHCLNGKRCHFSHNYFEWPPHALLVRQNFMLNRILKSMDKSIDTLSEISGAAEL
DRTEEYALGVVGVLESYIGSINNITKQSACVAMSKLLTELNSDDIKKLRDNEEPNSPKIRVYNTVISYI
ESNRKNNKQTIHLLKRLPADVLKKTIKTTLDIHKSITINNPKESTVSDINDHAKNNDTT
SEQ ID NO: 44 (nucleotide sequence for RSV M2-1 protein, NCBI Reference
Sequence: NC_001803.1)
ATGTCACGAAGGAATCCTTGCAAATTTGAAATTCGAGGTCATTGCTTGAATGGTAAGAGGTGT
CATTTTAGTCATAATTATTITGAATGGCCACCCCATGCACTGCTTGTAAGACAAAACTTTATGTT
AAACAGAATACTTAAGTCTATGGATAAAAGCATCGATACTTTATCAGAAATAAGTGGAGCTGC
AGAGTTGGACAGAACAGAAGAGTATGCCCTCGGTGTAGTTGGAGTGCTAGAGAGTTATATAG
GATCTATAAATAATATAACTAAACAATCAGCATGTGTTGCCATGAGCAAACTCCTCACTGAACT
CAACAGTGATGACATCAAAAAACTGAGGGACAATGAAGAGCCAAATTCACCCAAGATAAGAG
TGTACAATACTGTCATATCATATATTGAAAGCAACAGGAAAAACAATAAACAAACTATCCATCT
GTTAAAAAGATTGCCAGCAGACGTATTGAAGAAAACCATAAAAACCACATTGGATATCCACAA
GAGCATAACCATCAATAACCCAAAAGAATCAACTGTTAGTGATATAAACGACCATGCCAAAAA
TAATGATACTACCTGA
SEQ ID NO: 45 (amino acid sequence for RSV M2-1 protein codon-optimized
(A strain))
MSRRNPCKFEIRGHCLNGKRCHFSHNYFEWPPHALLVRQNFMLNRILKSMDKSIDTLSEISGAAEL
DRTEEYALGVVGVLESYIGSINNITKQSACVAMSKLLTELNSDDIKKLRDNEELNSPKIRVYNTVISYIE
SNRKNNKQTIHLLKRLPADVLKKTIKNTLDIHKSITINNPKESTVSDTNDHAKNNDTT
SEQ ID NO: 46 (nucleotide sequence for RSV M2-1 protein codon-optimized
(A strain))
ATGAGCCGGCGGAACCCCTGCAAGTTCGAGATCCGGGGCCACTGCCTGAACGGCAAGCGGTGCCACTTCAGCCAC
AACTACTTCGAGTGGCCCCCTCACGCCCTGCTGGTGCGCCAGAACTTCATGCTGAACCGGATCCTGAAGTCCATGG
ACAAGAGCATCGACACCCTGAGCGAGATCAGCGGAGCTGCCGAGCTGGACCGGACCGAGGAATATGCCCTGGGC
GTGGTGGGAGTGCTGGAAAGCTACATCGGCAGCATCAACAACATCACCAAGCAGAGCGCCTGCGTGGCCATGAG
CAAGCTGCTGACCGAGCTGAACAGCGACGACATCAAGAAGCTGCGGGACAACGAGGAACTGAACAGCCCCAAGA
TCCGGGTGTACAACACCGTGATCAGCTACATCGAGAGCAACCGGAAGAACAACAAGCAGACCATCCATCTGCTGA
AGCGGCTGCCCGCCGACGTGCTGAAGAAAACCATCAAGAACACCCTGGACATCCACAAGTCCATCACCATCAACA
ACCCCAAAGAAAGCACCGTGTCCGACACCAACGACCACGCCAAGAACAACGACACCACCTGA
SEQ ID NO: 47 (amino acid sequence for RSV P protein, NCBI Reference
Sequence: NC_001803.1)
MEKFAPEFHGEDANNRATKFLESIKGKFTSPKDPKKKDSIISVNSIDIEVTKESPITSNSTIINPTNETD
DTVGNKPNYQRKPLVSFKEDPTPSDNPFSKLYKETIETFDNNEEESSYSYEEINDQTNDNITARLDRI
DEKLSEILGMLHTLVVASAGPTSARDGIRDAMVGLREDMIEKIRTEALMTNDRLEAMARLRNEESE
KMAKDTSDEVSLNPTSEKLNNLLEGNDSDNDLSLDDF
SEQ ID NO: 48 (nucleotide sequence for RSV P protein, NCBI Reference
Sequence: NC_001803.1)
ATGGAAAAGTTTGCTCCTGAATTCCATGGAGAAGATGCAAACAACAGAGCTACCAAATTCCTA
GAATCAATAAAGGGCAAATTCACATCACCTAAAGATCCCAAGAAAAAAGATAGTATCATATCT
GTCAACTCAATAGATATAGAAGTAACCAAAGAAAGCCCTATAACATCAAATTCAACCATTATAA
ACCCAACAAATGAGACAGATGATACTGTAGGGAACAAGCCCAATTATCAAAGAAAACCTCTAG
TAAGTTTCAAAGAAGACCCTACGCCAAGTGATAATCCCTTTTCAAAACTATACAAAGAAACCAT
AGAAACATTTGATAACAATGAAGAAGAATCTAGCTATTCATATGAAGAAATAAATGATCAGAC
AAACGATAATATAACAGCAAGATTAGATAGGATTGATGAAAAATTAAGTGAAATACTAGGAAT
GCTTCACACATTAGTAGTAGCGAGTGCAGGACCTACATCTGCTCGGGATGGTATAAGAGATGC
CATGGTTGGTTTAAGAGAAGACATGATAGAAAAAATCAGAACTGAAGCATTAATGACCAATGA
CAGACTAGAAGCTATGGCAAGACTCAGGAATGAGGAAAGTGAAAAGATGGCAAAAGACACAT
CAGATGAAGTGTCTCTCAATCCAACATCAGAGAAATTGAACAACCTGTTGGAAGGGAATGATA
GTGACAATGATCTATCACTTGATGATTTCTGA
SEQ ID NO: 49 (amino acid sequence for RSV P protein codon-optimized (A
strain))
MEKFAPEFHGEDANNRATKFLESIKGKFTSPKDPKKKDSIISVNSIDIEVTKESPITSNSTIINPTNETD
DTAGNKPNYQRKPLVSFKEDPTPSDNPFSKLYKETIETFDNNEEESSYSYEEINDQTNDNITARLDRI
DEKLSEILGMLHTLVVASAGPTSARDGIRDAMIGLREEMIEKIRTEALMTNDRLEAMARLRNEESE
KMAKDTSDEVSLNPTSEKLNNLLEGNDSDNDLSLEDF
SEQ ID NO: 50 (nucleotide sequence for RSV P protein codon-optimized (A
strain))
ATGGAAAAGTTCGCCCCCGAGTTCCACGGCGAGGACGCCAACAACCGGGCCACCAAGTTTCTG
GAATCCATCAAGGGCAAGTTCACCAGCCCCAAGGACCCCAAGAAGAAGGACAGCATCATCAGC
GTGAACAGCATCGACATCGAAGTGACCAAAGAGAGCCCCATCACCAGCAACAGCACCATCATC
AACCCCACCAACGAGACAGACGACACCGCCGGCAACAAGCCCAACTACCAGCGGAAGCCCCTG
GTGTCCTTCAAAGAGGACCCCACCCCCAGCGACAACCCCTTCAGCAAGCTGTACAAAGAGACA
ATCGAGACATTCGACAACAACGAGGAAGAGAGCAGCTACAGCTACGAGGAAATCAACGACCA
GACCAACGACAACATCACCGCCAGACTGGACCGGATCGACGAGAAGCTGAGCGAGATCCTGG
GCATGCTGCACACCCTGGTGGTGGCCTCTGCCGGCCCTACAAGCGCCAGAGATGGCATCCGGG
ACGCCATGATCGGCCTGCGGGAAGAGATGATCGAGAAGATCCGGACCGAGGCCCTGATGACC
AACGACCGGCTGGAAGCCATGGCCCGGCTGCGGAACGAGGAATCCGAGAAGATGGCCAAGG
ACACCAGCGACGAGGTGTCCCTGAACCCCACCTCTGAGAAGCTGAACAACCTGCTGGAAGGCA
ACGACAGCGACAACGACCTGAGCCTGGAAGATTTCTGA

1. A virus like particle (VLP) comprising a respiratory syncytial virus (RSV) M protein and an RSV M2-1 protein.

2. The VLP of claim 1, wherein the VLP comprises one or more additional RSV proteins.

3. The VLP of claim 1, wherein the VLP comprises an RSV F protein.

4. The VLP of claim 3, wherein the RSV F protein is selected from a group consisting of a pre-fusion form of the RSV F protein, a post-fusion form of the RSV F protein, and a carbonyl terminal portion of the RSV F protein.

5. The VLP of claim 3, wherein the RSV F protein comprises a sequence selected from the group consisting of SEQ ID NO: 23, SEQ ID NO: 26, SEQ ID NO: 29, SEQ ID NO: 32, SEQ ID NO: 34, SEQ ID NO: 36, and SEQ ID NO: 37.

6. The VLP of claim 3, wherein the carbonyl terminal portion of the RSV F protein comprises a sequence of SEQ ID NO: 32.

7. The VLP of claim 1, wherein the VLP comprises an RSV G protein.

8. The VLP of claim 7, wherein the RSV G protein is from RSV group A or RSV group B.

9. The VLP of claim 7, wherein the RSV G protein comprises a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21.

10. The VLP of claim 1, wherein the VLP comprises a recombinant RSV G protein.

11. The VLP of claim 10, wherein the recombinant RSV G protein comprises a transmembrane domain of an RSV G protein and a central conserved domain of the RSV G protein.

12. A vaccine comprising the VLP of claim 1.

13. The vaccine of claim 12, further comprising an adjuvant.

14. A method of inducing immunity to RSV infection or at least one symptom thereof in a subject, comprising administering one or more effective doses of the vaccine of claim 12.

15. The method of claim 14, wherein the one or more effective doses of the vaccine are administered to the subject via a route that is selected from the group consisting of an intramuscular route, a subcutaneous route, an intradermal route, an oral administration, a nasal administration, and inhalation.

16.-27. (canceled)
